(19) United States
(12) Patent Application Publication
(10) Pub. No.: US 2026/0294568 A1
(43) Pub. Date: Oct. 1, 2026

(54) HOST AND ROBOT COMMUNICATION FOR A CONTINUUM ROBOT OR ENDOSCOPIC DEVICE OR SYSTEM

(71) Applicant: Canon U.S.A., Inc., Melville, NY (US)

(72) Inventors: Brian Fitch Williams, Watertown, MA (US); Andrew Frank Vesper, Townsend, MA (US)

(21) Appl. No.: 19/095,937

(22) Filed: Mar. 31, 2025

(51) Int. Cl. A61B 34/37; A61B 34/00; A61B 34/30; A61B 34/32

(52) U.S. Cl. CPC A61B 34/37; A61B 34/25; A61B 34/32; A61B 2034/301

(57) ABSTRACT

One or more devices, systems, methods, and storage mediums for performing host and robot communication, error avoidance or removal communication, autonomous driving/navigation/planning, movement detection, and/or control for a continuum robot are provided herein. Examples include, but are not limited to, navigation planning and/or autonomous navigation/state changes of one or more portions of a continuum robot towards a particular target, monitoring/controlling host apparatus and robot controlling apparatus/actuator communication, and/or removal of error(s) in such communication(s). Examples of applications include imaging, evaluating, and diagnosing biological objects, such as, but not limited to, for Gastro-intestinal, cardio, bronchial, and/or ophthalmic applications, and being obtained via one or more optical instruments, such as, but not limited to, optical probes, catheters, endoscopes, and bronchoscopes. Techniques provided herein also improve processing and imaging efficiency while achieving images that are more precise, and also achieve reduced mental and physical burden and improved ease of use.

### FIELD OF THE DISCLOSURE

The present disclosure generally relates to imaging and, more particularly, to bronchoscope(s), robotic bronchoscope(s), robot apparatus(es), method(s), and storage medium(s) that operate to image a target, object, or specimen (such as, but not limited to, a lung, a biological object or sample, tissue, etc.) and/or to a continuum robot apparatus, method, and storage medium to implement robotic control by providing accurate information to a user about a posture/position/state/etc. for a catheter or bendable robot and/or for all sections of the catheter/bendable robot or imaging device/apparatus or system to perform navigation planning and/or autonomous driving or navigation and/or to match a state or states when each section reaches or approaches a same or similar, or approximately a same or similar, state or states of a first section of the catheter or imaging device, apparatus, or system. One or more bronchoscopic, endoscopic, medical, camera, catheter, or imaging devices, systems, and methods and/or storage mediums for use with same, are discussed herein. One or more devices, methods, or storage mediums may be used for medical applications and, more particularly, to steerable, flexible medical devices that may be used for or with guide tools and devices in medical procedures, including, but not limited to, endoscopes, cameras, and catheters.

### BACKGROUND

Endoscopy, bronchoscopy, catheterization, and other medical procedures facilitate the ability to look inside a body. During such a procedure, a flexible medical tool may be inserted into a patient's body, and an instrument may be passed through the tool to examine or treat an area inside the body. For example, a bronchoscope is an endoscopic instrument to view inside the airways of a patient. Catheters and other medical tools may be inserted through a tool channel in the bronchoscope to provide a pathway to a target area in the patient for diagnosis, planning, medical procedure(s), treatment, etc.

Robotic bronchoscopes, robotic endoscopes, or other robotic imaging devices may be equipped with a tool channel or a camera and biopsy tools, and such devices (or users of such devices) may insert/retract the camera and biopsy tools to exchange such components. The robotic bronchoscopes, endoscopes, or other imaging devices may be used in association with a display system and a control system.

An imaging device, such as a camera, may be placed in the bronchoscope, the endoscope, or other imaging device/system to capture images inside the patient and to help control and move the bronchoscope, the endoscope, or the other type of imaging device, and a display or monitor may be used to view the captured images. An endoscopic camera that may be used for control may be positioned at a distal part of a catheter or probe (e.g., at a tip section).

The display system may display, on the monitor, an image or images captured by the camera, and the display system may have a display coordinate used for displaying the captured image or images. In addition, the control system may control a moving direction of the tool channel or the camera. For example, the tool channel or the camera may be bent according to a control by the control system. The control system may have an operational controller (such as, but not limited to, a joystick, a gamepad, a controller, an input device, etc.), and physicians may rotate or otherwise move the camera, probe, catheter, etc. to control same. However, such control methods or systems are limited in effectiveness. Indeed, while information obtained from an endoscopic camera at a distal end or tip section may help decide which way to move the distal end or tip section, such information does not provide details on how the other bending sections or portions of the bronchoscope, endoscope, or other type of imaging device may move to best assist the navigation.

The reliance on human operators to guide a robotic system introduces potential variability in sampling accuracy and operator-dependent outcomes. Such operators may introduce human error, reduce efficiency of using a robotic system, have a steeper learning curve to using a robotic system, and affect surgeries as a result.

Additionally, where a host controller or processor operates to generate a Graphical User Interface (GUI) based on information of instructions from a robot controller or processor, it is possible that inaccurate information about a posture (or other orientation/state information) of a robotic device or system may be provided in a case where the host controller hangs or crashes and the GUI is not generated accurately. This would lead to a user of the robotic device or system receiving the inaccurate information and/or an error about the posture of the robotic device or system and may cause the user to input inappropriate/inaccurate instruction(s) to the robotic device or system.

As such, there is a need for devices, systems, methods, and/or storage mediums that accurately provide the feature(s) or information details on how the other bending sections or portions of such imaging devices, imaging systems, etc. (e.g., endoscopic devices, bronchoscopes, other types of imaging devices/systems, etc.) may move to best assist navigation and/or state or state(s) for same, to keep track of a path of a tip of the imaging devices, imaging systems, etc., and there is a need for a more appropriate and accurate navigation and communication regarding same of a robotic device or system while providing accurate information to a user of the robotic device or system.

Accordingly, it would be desirable to provide at least one imaging, optical, or control device, system, method, and storage medium for controlling one or more endoscopic or imaging devices or systems, for example, by using more accurate host and robot communication, by using error communication to avoid or eliminate errors or inaccurate information, and/or by implementing automatic (e.g., robotic) or manual control of each portion or section of the at least one imaging, optical, or control device, system, method, and storage medium to keep track of and to match the state or state(s) of a first portion or section in a case where each portion or section reaches or approaches a same or similar, or approximately same or similar, state or state(s) and to provide a more appropriate and accurate navigation of a robotic device or system.

### SUMMARY

Accordingly, it is a broad object of the present disclosure to provide imaging (e.g., computed tomography (CT), Magnetic Resonance Imaging (MRI), etc.) apparatuses, systems, methods, and storage mediums for using host and robot communication, for using error communication to avoid or eliminate one or more errors when controlling a robotic apparatus or system, and/or for using a navigation and/or control method or methods (manual or automatic) in one or more apparatuses or systems (e.g., an imaging apparatus or system, an endoscopic imaging device or system, etc.). It is also a broad object of the present disclosure to provide imaging (e.g., computed tomography (CT), Magnetic Resonance Imaging (MRI), etc.) apparatuses, systems, methods, and storage mediums for using host and robot communication, for using error communication to avoid or eliminate one or more errors when controlling a robotic apparatus or system, and/or for using a navigation and/or control method or methods for achieving navigation planning, autonomous navigation, and/or control through a target, sample, or object (e.g., lung airway(s) during bronchoscopy, a lumen of the target/sample/object, etc.) in one or more apparatuses or systems (e.g., an imaging apparatus or system, an endoscopic imaging device or system, etc.). To address one or more of the above issues, the present disclosure provides novel host and robot communication features, novel error communication features, and novel supervised-autonomous driving approach(es) that integrate a novel depth-based airway tracking method(s) and a robotic bronchoscope.

In one or more embodiments, a robot controlling apparatus or actuator may include: one or more processors that operate to: check a status of one or more communications of the robot controlling apparatus or actuator with a host processor of a host apparatus; determine whether an error is detected in the one or more communications with the host processor; and in a case where the error is detected in the one or more communications with the host processor, stop a bendable robot or catheter from moving, bending, or changing a state of the bendable robot or catheter. In one or more embodiments, one or more of the following may occur: (i) the robot controlling apparatus or actuator may be connected to or in communication with an input device via a first interface of the robot controlling apparatus or actuator, the input device operating to transmit one or more instructions to the one or more processors of the robot controlling apparatus or actuator where the one or more instructions operate to move, control, bend, or change the state of the bendable robot or catheter; and (ii) the robot controlling apparatus or actuator may be connected to or in communication with the host apparatus via a second interface of the robot controlling apparatus or actuator such that the host processor of the host apparatus and the one or more processors of the robot controlling apparatus or actuator transmit the one or more communications and/or transmit the one or more instructions from the input device to the host processor of the host apparatus. In one or more embodiments, one or more of the following may occur: (i) the host processor of the host apparatus operates to generate a Graphical User Interface (GUI) based on information of the one or more instructions from the input device and/or to display posture, position, orientation, bending, or other state information of the bendable robot or catheter; (ii) the host processor of the host apparatus operates to generate a Graphical User Interface (GUI) based on information of the one or more instructions from the input device and/or to display current posture, position, orientation, bending, or other state information of the bendable robot or catheter; (iii) the robot controlling apparatus or actuator is connected to or in communication with a display that operates to display a Graphical User Interface (GUI) to display posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately; (iv) the robot controlling apparatus or actuator is connected to or in communication with a display that operates to display a Graphical User Interface (GUI) to display current posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately; and/or (v) in the case where the error is detected in the one or more communications with the host processor, the robot controlling apparatus or actuator and/or the bendable robot or catheter is/are put in a safety shutdown mode to avoid misleading a user of the robot controlling apparatus or actuator and/or the bendable robot or catheter since the user cannot see result(s) of one or more instructions or actions of the user.

In the case where the robot controlling apparatus or actuator is connected to or in communication with the host apparatus via the second interface of the robot controlling apparatus or actuator, one or more of the following may occur: (i) the one or more processors of the robot controlling apparatus or actuator further operate to send one or more of the following to the host processor of the host apparatus: state information of the bendable robot or catheter; state information of the bendable robot or catheter indicating whether the bendable robot or catheter is connected to the robot controlling apparatus or actuator or not; state information or navigation information of the bendable robot or catheter as a tip of the bendable robot or catheter and/or a linear stage connected to the bendable robot or catheter is or are manipulated, controlled, or moved; one or more requests from a user of the robot controlling apparatus or actuator or change the state of the bendable robot or catheter; one or more requests from the user to take one or more images or snapshots using a camera of the bendable robot or catheter; one or more requests from the user to rotate the one or more images or snapshots of the camera of the bendable robot or catheter; information for a warning indicating the error; information for a warning, failure, or error related to a voltage being out of a set or predetermined range, a temperature being out of a set or predetermined range, and/or an emergency stop; version information and parameter values; and/or information automatically sent to the host processor or information manually sent to the host processor in response to a request for the information; (ii) the host processor of the host apparatus operates to send one or more of the following to the one or more processors of the robot controlling apparatus or actuator: one or more requests for state, version, voltage, temperature, and/or parameter information; one or more requests to home or to return to a default, predetermined, or rest position/state a linear stage being used to move the bendable robot or catheter; one or more requests to zero or reset one or more motors of the robot controlling apparatus or actuator that operate to change the state of the bendable robot or catheter; one or more requests to change the state of the bendable robot or catheter and/or the linear stage; information for a test or normal joystick mode; error report(s); a request for shutdown or stopping of the bendable robot or catheter and/or the robot controlling apparatus or actuator; and/or Electromagnetic Compatibility (EMC) testing parameters and operations; and/or (iii) the host processor of the host apparatus operates to perform one or more of the following: generation of a Graphical User Interface (GUI) based on information of the one or more instructions from the input device and/or to display current posture, position, orientation, bending, or other state information of the bendable robot or catheter; display of the GUI on a display; one or more pre-procedure operations related to login and/or entering case information; one or more procedure setup operations; one or more procedure operations related to displaying video from a camera of the bendable robot or catheter, showing the state or statue of the bendable robot or catheter, showing the state or statue of the bendable robot or catheter including one or more forces affecting one or more drive wires of the bendable robot or catheter, showing one or more warnings related to displaying the error information, and/or showing additional video feed(s); post-procedure operations related to reviewing cases and/or exporting the cases to a memory for storage; one or more management operations related to adding or removing user(s) of the robot controlling apparatus or actuator, the input device, and/or of the host apparatus, and/or related to handling one or more settings for each of the user(s); and/or writing or storing a log to a disk or to the memory.

In one or more embodiments, the robot controlling apparatus or actuator may further include or be in communication with one or more indicators that operate to indicate that the error is happening or has happened, in response to the detection of the error in the one or more communications with the host processor, wherein one or more of the following may occur: (i) the one or more indicators independently or automatically signal that the error is happening or has happened, or the one or more indicators independently or automatically signal that the error is happening or has happened to a user of the robot controlling apparatus or actuator and/or the input device; (ii) the one or more indicators are disposed in or on one or more of the following: the robot controlling apparatus or actuator, the input device, the host apparatus, and/or a display that operates to display a Graphical User Interface (GUI); and/or (iii) the bendable robot or catheter, the input device, the host apparatus, and/or the robot controlling apparatus or actuator operates to independently signal that the error is happening or has happened via the one or more indicators as a primary or secondary signal of the error.

In one or more embodiments, the robot controlling apparatus or actuator may further include or be in communication with one or more one or more motors, wherein one or more of the following may occur: (i) the one or more processors of the robot controlling apparatus or actuator further operate to control the one or more motors to move, control, bend, or change the state of the bendable robot or catheter; (ii) the robot controlling apparatus or actuator is connected to or in communication with the bendable robot or catheter such that the one or more processors and the one or more motors of the robot controlling apparatus operate to move, control, bend, or change the state of the bendable robot or catheter; and/or (iii) in the case where the error is detected in the one or more communications with the host processor, the one or more processors of the robot controlling apparatus or actuator further operate to restrict, disable, or stop the one or more motors from moving, controlling, bending, or changing the state of the bendable robot or catheter. In one or more embodiments, the one or more processors of the robot controlling apparatus or actuator may further operate to restrict, disable, or stop the one or more motors from moving, controlling, bending, or changing the state of the bendable robot or catheter in a case where the one or more processors of the robot controlling apparatus or actuator do not receive a predetermined response and/or a valid message from the host processor of the host apparatus within a predetermined period or amount of time, where the predetermined response and/or the valid message may include one or more of the following: a query version string; a request to reset the bendable robot or catheter; a request to run power-on test(s); a request or query for a cyclic redundancy check (CRC) and/or for an on or off status of the CRC; a parameter(s) query; a request to query, notify, or set a failure string; a request to query, notify, or set a warning string; a request to notify or log a string or log string to an application log; a request to shut down the bendable robot or catheter and/or the robot controlling apparatus or actuator; a request to start a new procedure; a request to power cycle; a request for a query of complete status; a request to query, notify or set a mode, a disconnect mode, a disabled mode, a halt mod, a tip control mode, a manual mode, a pause mode, a relax mode, a park mode, a Follow-the-Leader (FTL) mode, a reverse FTL mode, a targeting mode, and/or an error mode; a request to query or notify an emergency stop or E-stop, or to obtain an ok or tripped status of the emergency stop; a request to establish a home, zero, or default position for the bendable robot or catheter for one or more or all motors or drive wires of the robot controlling apparatus or actuator; a request to query or notify a connected or a disconnected status of the bendable robot or catheter; and/or a request for a wire test of the bendable robot or catheter.

In one or more embodiments, one or more of the following may occur: (i) the one or more processors of the robot controlling apparatus or actuator further operate to determine whether the error is detected in the one or more communications with the host processor by using or communicating with one or more of the following: a driver or an operating system driver of the robot controlling apparatus or actuator, firmware of the robot controlling apparatus or actuator, software of the robot controlling apparatus or actuator, a robot state from the host processor of the host apparatus, and/or a serial communications and protocol manager of the host processor of the host apparatus; (ii) the one or more processors of the robot controlling apparatus or actuator further operate to control an operation mode of the bendable robot or catheter to be in a relaxed mode in the case where the error is detected in the one or more communications with the host processor, or the one or more processors of the robot controlling apparatus or actuator further operate to control an operation mode of the bendable robot or catheter to be in a relaxed mode in the case where the error is detected in the one or more communications with the host processor where the bendable robot or catheter bends or changes the state of the bendable robot or catheter based on or in accordance with an external force so that the bendable robot or catheter conforms to a lumen of an object, target, or sample in a case where the bendable robot or catheter is removed from the lumen of the object, target, or sample; and/or (iii) the one or more processors of the robot controlling apparatus or actuator further operate to control an operation mode of the bendable robot or catheter to be in a relaxed mode in the case where the error is detected in the one or more communications with the host processor such that the relaxed mode operates as a safety mode where the bendable robot or catheter is removed from an object, target, or sample.

In one or more embodiments, the robot controlling apparatus or actuator may further include or be in communication with a slider, a rail, and/or a translational stage that operates to move the robot controlling apparatus or actuator and/or the bendable robot or catheter to provide depth control or remember a path along or into a lumen of a target, sample, or object, wherein one or more of the following may occur: (i) the one or more processors of the robot controlling apparatus or actuator and/or the host processor further operate(s) to provide the depth control or to remember the path and to move the bendable robot or catheter using one or more of the following: a Follow-The-Leader (FTL) algorithm or process to move one or more sections of the bendable robot or catheter along the path; and/or a reverse Follow-The-Leader (rFTL) algorithm or process to reverse the path exactly or to re-orient the one or more sections of the bendable robot or catheter in a manner in which there is less impedance in the bending used to align the bendable robot or catheter with the lumen of the target, sample, or object; and/or (ii) the one or more processors of the robot controlling apparatus or actuator and/or the host processor further operate(s) to, in the case where the error is detected in the one or more communications with the host processor, stop the movement of the robot controlling apparatus or actuator and/or the bendable robot or catheter via the slider, the rail, and/or the translational stage; or the one or more processors of the robot controlling apparatus and/or the host processor further operate(s) to, in the case where the error is detected in the one or more communications with the host processor, stop the movement of the robot controlling apparatus or actuator and/or the bendable robot or catheter via the slider, the rail, and/or the translational stage such that a user of the robot controlling apparatus or actuator, of the host apparatus, and/or of the bendable robot or catheter is stopped or prevent from driving the bendable robot or catheter further into the lumen with no visibility or information of action(s) of the user. In one or more embodiments, the one or more processors of the robot controlling apparatus or actuator and/or the host processor may further operate to one or more of the following: (i) estimate or determine a depth of the depth control and/or estimate or determine the path to the target, sample, or object; (ii) estimate or determine a depth of the depth control and/or estimate or determine the path to the target, sample, or object using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture; and/or (iii) use a neural network, a convolutional neural network, a generative adversarial network (GAN), a consistent generative adversarial network (cGAN), a three cycle-consistent generative adversarial network (3cGAN), recurrent neural networks, and/or any other AI architecture to one or more of: use or evaluate an estimated or determined depth or path, identify one or more target points in the lumen or along the path, evaluate the accuracy of the identified one or more target points, plan the navigation of the bendable robot or catheter, autonomously move the bendable robot or catheter to the one or more target points, take one or more images, frames, or views using a camera of the bendable robot or catheter, and/or display the one or more target points on one of the one or more images, frames, or views on a display or indicate on the display or via an indicator on the display or via a light or indicator on or in communication with the bendable robot or catheter that the navigation has been planned or determined.

In one or more embodiments, the robot controlling apparatus or actuator may further include or be in communication with a camera that operates to be inserted into a lumen or be disposed at a distal end of the bendable robot or catheter to obtain one or more images or a live video feed for reference and/or for navigation in a lumen of an object, target, or sample, wherein one or more of the following may occur: (i) the one or more images or the live video feed is displayed on a display; and/or (ii) the host processor further operates to keep the one or more images or the live video feed being displayed even in the case where the error is detected to allow for operation, movement, and/or removable of the bendable robot or catheter.

In one or more embodiments, one or more of the following may occur: (i) the bendable robot or catheter includes a distal bending section or portion, wherein the distal bending section or portion is commanded or instructed automatically or based on an input of a user of the bendable robot or catheter; (ii) the bendable robot or catheter includes a plurality of bending sections or portions including a distal or most distal bending portion or section and the rest of the plurality of the bending sections or portions, where the plurality of bending sections or portions each include driving wires that operate to bend a respective section or portion of the plurality of sections or portions, and where the driving wires are connected to the robot controlling apparatus or actuator or to respective one or more motors of the robot controlling apparatus or actuator so that the robot controlling apparatus or actuator operates to bend one or more of the plurality of bending sections or portions using the driving wires; and/or (iii) the robot controlling apparatus further comprises a slider, a rail, and/or a translational stage, and the one or more processors further operate to instruct or command the forward motion, or the motion in a set or predetermined direction, of the slider, the rail, the translational stage, and/or the bendable robot or catheter automatically or autonomously and/or based on an input of a user of the bendable robot or catheter.

Additionally, one or more aspects of the present disclosure may include or relate to one or more algorithms or processes that operate to command independently or simultaneously, or issue simultaneous commands, to: (i) bend a robot, continuum robot, robotic catheter, and/or one or more components or sections thereof; and (ii) move a stage, linear stage, and/or one or more components of or attached to a stage or linear stage. As such, one or more embodiments may simultaneously bend a robot, continuum robot, robotic catheter, one or more components or sections of a continuum robot or catheter, etc. and move a stage, linear stage, one or more components of a stage or linear stage, etc. In one or more embodiments, by independently or simultaneously handling the tasks of advancing and centering the robot, probe, catheter, robotic bronchoscope, etc. in a target (e.g., in a lung or airway, in a lumen of an object/target/sample, etc.), the method(s) of the present disclosure may assist physicians in providing accurate control information and may assist the physicians in concentrating on the clinical decision-making to reach the target, which achieves or provides enhancements to the efficacy of such imaging, bronchoscopy, endoscopy, etc.

One or more devices, systems, methods, and storage mediums for using communication feature(s) and/or for navigation planning and/or performing control/navigation, including of a multi-section continuum robot and/or for viewing, imaging, and/or characterizing tissue and/or lesions, or an object or sample, using one or more imaging techniques (e.g., robotic bronchoscope imaging, bronchoscope/endoscope/catheter imaging, etc.) or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure are described diagrammatically and visually in the figures included herewith.

In one or more embodiments, the one or more processors may further operate to determine one or more geometry metrics, wherein the one or more geometry metrics is a depth map or maps obtained or generated by processing one or more images or frames or one or more view of the camera, and the processors may further operate to one or more of the following: advance the continuum robot or catheter to the target point; display one or more target points on a display; detect one or more objects, and fit one or more set or predetermined geometric shapes or one or more circles, rectangles, squares, ovals, octagons, and/or triangles in or on one or more detected objects; define the one or more target points based on the one or more set or predetermined geometric shapes or based on the one or more circles, rectangles, squares, ovals, octagons, and/or triangles of the one or more detected objects; set a center or portion of the circle or circles as the one or more target points for a next movement of the continuum robot; in a case where the one or more target points are not detected, then apply peak detection to the depth map or maps and use one or more detected peaks as the one or more targets; and/or in a case where one or more peaks are not detected, then use a deepest point of the depth map or maps as the one or more targets. The one or more processors may further operate to one or more of the following: (i) estimate or determine the depth map or maps using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein; and/or (ii) use a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein to one or more of: select a target detection method or mode; use or evaluate a depth map or depth maps; perform the selected target detection method or mode; identify one or more target points; evaluate the accuracy of the identified one or more target points; and/or plan the navigation of the continuum robot, autonomously move the continuum robot to the one or more target points, or display the one or more target points on one of the one or more images, frames, or views on a display or indicate on the display or via an indicator on the display or via a light or indicator on the continuum robot that the navigation plan has been planned or determined.

In one or more embodiments, the target point(s) may be located in a lung or in an airway, and the continuum robot may be a bronchoscope.

The one or more processors may further operate to perform registration or co-registration for changes due to movement, breathing, or any other change that may occur during imaging or a procedure with the continuum robot. The continuum robot may be a steerable catheter with a camera at a distal end of the steerable catheter. The continuum robot may include one or more of the following: (i) a distal bending section or portion, wherein the distal bending section or portion is commanded or instructed automatically or based on an input of a user of the continuum robot; (ii) a plurality of bending sections or portions including a distal or most distal bending portion or section and the rest of the plurality of the bending sections or portions; and/or (iii) the one or more processors further operate to instruct or command the forward motion, or the motion in the set or predetermined direction, of the translational stage and/or of the continuum robot automatically or autonomously and/or based on an input of a user of the continuum robot. The continuum robots may further include: a base and an actuator that operates to bend the plurality of the bending sections or portions independently; and the translational stage being a motorized linear stage and/or a sensor or camera that operates to move the continuum robot forward and backward, and/or in the predetermined or set direction or directions, wherein the one or more processors may operate to control the actuator and the motorized linear stage and/or the sensor or camera. The continuum robot(s) may further include a user interface of or disposed on a base, or disposed remotely from a base, the user interface operating to receive an input from a user of the continuum robot to move one or more of the plurality of bending sections or portions and/or a motorized linear stage as the translational stage and/or a sensor or camera, wherein the one or more processors further operate to receive the input from the user interface, and the one or more processors and/or the user interface operate to use a base coordinate system. The plurality of bending sections or portions may each include driving wires that operate to bend a respective section or portion of the plurality of sections or portions, wherein the driving wires may be connected to an actuator so that the actuator operates to bend one or more of the plurality of bending sections or portions using the driving wires. One or more of the following may occur: (i) the continuum robot further comprises an operational controller or joystick that operates to issue or input one or more commands or instructions as an input to the one or more processors, the input including an instruction or command to move one or more of a plurality of bending sections or portions and/or a motorized linear stage and/or a sensor or camera; (ii) the continuum robot further includes a display to display one or more images taken by the continuum robot and/or to display a navigation plan and/or an autonomous navigation path of the continuum robot; and/or (iii) the continuum robot further comprises an operational controller or joystick that operates to issue or input one or more commands or instructions to one or more processors, the input including an instruction or command to move one or more of a plurality of bending sections or portions and/or a motorized linear stage and/or a sensor or camera, and the operational controller or joystick operates to be controlled by a user of the continuum robot.

In one or more embodiments, a method for performing error detection and/or error removal for a robot controlling apparatus/actuator and/or a bendable robot or catheter may include: checking a status of one or more communications of one or more processors of the robot controlling apparatus or actuator with a host processor of a host apparatus; determining whether an error is detected in the one or more communications with the host processor; and in a case where the error is detected in the one or more communications with the host processor, stopping a bendable robot or catheter from moving, bending, or changing a state of the bendable robot or catheter. One or more methods may further include any combination of continuum robot feature(s) or other method feature(s) discussed herein. One or more methods may further include one or more of the following: (i) transmitting one or more instructions from an input device to the one or more processors of the robot controlling apparatus or actuator where the one or more instructions operate to move, control, bend, or change the state of the bendable robot or catheter; and/or (ii) transmitting the one or more communications and/or transmitting the one or more instructions from the input device to the host processor of the host apparatus. One or more methods may further include one or more of the following: (i) generating, via the host processor, a Graphical User Interface (GUI) based on information of the one or more instructions from the input device, and/or displaying posture, position, orientation, bending, or other state information of the bendable robot or catheter; (ii) generating, via the host processor, a Graphical User Interface (GUI) based on information of the one or more instructions from the input device, and/or displaying current posture, position, orientation, bending, or other state information of the bendable robot or catheter; (iii) displaying a Graphical User Interface (GUI) to display posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately; (iv) displaying a Graphical User Interface (GUI) to display current posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately; and/or (v) in the case where the error is detected in the one or more communications with the host processor, putting the robot controlling apparatus or actuator and/or the bendable robot or catheter in a safety shutdown mode to avoid misleading a user of the robot controlling apparatus or actuator and/or the bendable robot or catheter since the user cannot see result(s) of one or more instructions or actions of the user. One or more methods may further include on or more of the following: (i) sending, via the one or more processors, one or more of the following to the host processor of the host apparatus: state information of the bendable robot or catheter; state information of the bendable robot or catheter indicating whether the bendable robot or catheter is connected to the robot controlling apparatus or actuator or not; state information or navigation information of the bendable robot or catheter as a tip of the bendable robot or catheter and/or a linear stage connected to the bendable robot or catheter is or are manipulated, controlled, or moved; one or more requests from a user of the robot controlling apparatus or actuator or change the state of the bendable robot or catheter; one or more requests from the user to take one or more images or snapshots using a camera of the bendable robot or catheter; one or more requests from the user to rotate the one or more images or snapshots of the camera of the bendable robot or catheter; information for a warning indicating the error; information for a warning, failure, or error related to a voltage being out of a set or predetermined range, a temperature being out of a set or predetermined range, and/or an emergency stop; version information and parameter values; and/or information automatically sent to the host processor or information manually sent to the host processor in response to a request for the information; (ii) sending, via the host processor, one or more of the following to the one or more processors of the robot controlling apparatus or actuator: one or more requests for state, version, voltage, temperature, and/or parameter information; one or more requests to home or to return to a default, predetermined, or rest position/state a linear stage being used to move the bendable robot or catheter; one or more requests to zero or reset one or more motors of the robot controlling apparatus or actuator that operate to change the state of the bendable robot or catheter; one or more requests to change the state of the bendable robot or catheter and/or the linear stage; information for a test or normal joystick mode; error report(s); a request for shutdown or stopping of the bendable robot or catheter and/or the robot controlling apparatus or actuator; and/or Electromagnetic Compatibility (EMC) testing parameters and operations; and/or (iii) performing, via the host processor, one or more of the following: generation of a Graphical User Interface (GUI) based on information of the one or more instructions from the input device and/or to display current posture, position, orientation, bending, or other state information of the bendable robot or catheter; display of the GUI on a display; one or more pre-procedure operations related to login and/or entering case information; one or more procedure setup operations; one or more procedure operations related to displaying video from a camera of the bendable robot or catheter, showing the state or statue of the bendable robot or catheter, showing the state or statue of the bendable robot or catheter including one or more forces affecting one or more drive wires of the bendable robot or catheter, showing one or more warnings related to displaying the error information, and/or showing additional video feed(s); post-procedure operations related to reviewing cases and/or exporting the cases to a memory for storage; one or more management operations related to adding or removing user(s) of the robot controlling apparatus or actuator, the input device, and/or of the host apparatus, and/or related to handling one or more settings for each of the user(s); and/or writing or storing a log to a disk or to the memory.

One or more methods may further include: using one or more indicators that operate to indicate that the error is happening or has happened, in response to the detection of the error in the one or more communications with the host processor, wherein one or more of the following may occur: (i) the one or more indicators independently or automatically signal that the error is happening or has happened, or the one or more indicators independently or automatically signal that the error is happening or has happened to a user of the robot controlling apparatus or actuator and/or the input device; (ii) the one or more indicators are disposed in or on one or more of the following: the robot controlling apparatus or actuator, the input device, the host apparatus, and/or a display that operates to display a Graphical User Interface (GUI); and/or (iii) the bendable robot or catheter, the input device, the host apparatus, and/or the robot controlling apparatus or actuator operates to independently signal that the error is happening or has happened via the one or more indicators as a primary or secondary signal of the error.

One or more methods may further include: using or instructing one or more motors of the robot controlling apparatus or actuator to one or more of the following: (i) move, control, bend, or change the state of the bendable robot or catheter; and/or (ii) in the case where the error is detected in the one or more communications with the host processor and/or in a case where the one or more processors of the robot controlling apparatus or actuator do not receive a predetermined response and/or a valid message from the host processor of the host apparatus within a predetermined period or amount of time, restrict, disable, or stop the movement, control, bending, or changing of the state of the bendable robot or catheter, the predetermined response and/or the valid message including one or more of the following: a query version string; a request to reset the bendable robot or catheter; a request to run power-on test(s); a request or query for a cyclic redundancy check (CRC) and/or for an on or off status of the CRC; a parameter(s) query; a request to query, notify, or set a failure string; a request to query, notify, or set a warning string; a request to notify or log a string or log string to an application log; a request to shut down the bendable robot or catheter and/or the robot controlling apparatus or actuator; a request to start a new procedure; a request to power cycle; a request for a query of complete status; a request to query, notify or set a mode, a disconnect mode, a disabled mode, a halt mod, a tip control mode, a manual mode, a pause mode, a relax mode, a park mode, a Follow-the-Leader (FTL) mode, a reverse FTL mode, a targeting mode, and/or an error mode; a request to query or notify an emergency stop or E-stop, or to obtain an ok or tripped status of the emergency stop; a request to establish a home, zero, or default position for the bendable robot or catheter for one or more or all motors or drive wires of the robot controlling apparatus or actuator; a request to query or notify a connected or a disconnected status of the bendable robot or catheter; and/or a request for a wire test of the bendable robot or catheter. One or more methods may further include or have one or more of the following occur: (i) the determination of whether the error is detected in the one or more communications with the host processor uses or communicates with one or more of the following: a driver or an operating system driver of the robot controlling apparatus or actuator, firmware of the robot controlling apparatus or actuator, software of the robot controlling apparatus or actuator, a robot state from the host processor of the host apparatus, and/or a serial communications and protocol manager of the host processor of the host apparatus; (ii) the method further comprises determining the error is detected in the one or more communications with the host processor, and controlling an operation mode of the bendable robot or catheter to be in a relaxed mode and/or to be in a relaxed mode where the bendable robot or catheter bends or changes the state of the bendable robot or catheter based on or in accordance with an external force so that the bendable robot or catheter conforms to a lumen of an object, target, or sample in a case where the bendable robot or catheter is removed from the lumen of the object, target, or sample; and/or (iii) the method further comprises determining the error is detected in the one or more communications with the host processor, and controlling an operation mode of the bendable robot or catheter to be in a relaxed mode such that the relaxed mode operates as a safety mode where the bendable robot or catheter is removed from an object, target, or sample.

One or more methods may further include moving or controlling a slider, a rail, and/or a translational stage attached to the robot controlling apparatus or actuator and/or the bendable robot or catheter to provide depth control or to remember a path along or into a lumen of a target, sample, or object, and one or more of the following: (i) providing the depth control or remembering the path, and moving the bendable robot or catheter using one or more of the following: a Follow-The-Leader (FTL) algorithm or process to move one or more sections of the bendable robot or catheter along the path; and/or a reverse Follow-The-Leader (rFTL) algorithm or process to reverse the path exactly or to re-orient the one or more sections of the bendable robot or catheter in a manner in which there is less impedance in the bending used to align the bendable robot or catheter with the lumen of the target, sample, or object; and/or (ii) detecting the error, stopping the movement of the robot controlling apparatus or actuator and/or the bendable robot or catheter via the slider, the rail, and/or the translational stage; or stopping the movement of the robot controlling apparatus or actuator and/or the bendable robot or catheter via the slider, the rail, and/or the translational stage such that a user of the robot controlling apparatus or actuator, of the host apparatus, and/or of the bendable robot or catheter is stopped or prevent from driving the bendable robot or catheter further into the lumen with no visibility or information of action(s) of the user. One or more methods may further include one or more of the following: (i) estimating or determining a depth of the depth control and/or estimating or determining the path to the target, sample, or object; (ii) estimating or determining a depth of the depth control and/or estimating or determining the path to the target, sample, or object using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture; and/or (iii) using a neural network, a convolutional neural network, a generative adversarial network (GAN), a consistent generative adversarial network (cGAN), a three cycle-consistent generative adversarial network (3cGAN), recurrent neural networks, and/or any other AI architecture to one or more of: use or evaluate an estimated or determined depth or path, identify one or more target points in the lumen or along the path, evaluate the accuracy of the identified one or more target points, plan the navigation of the bendable robot or catheter, autonomously move the bendable robot or catheter to the one or more target points, take one or more images, frames, or views using a camera of the bendable robot or catheter, and/or display the one or more target points on one of the one or more images, frames, or views on a display or indicate on the display or via an indicator on the display or via a light or indicator on or in communication with the bendable robot or catheter that the navigation has been planned or determined. One or more methods may further include one or more of the following: (i) inserting a camera into a lumen or disposing the camera at a distal end of the bendable robot or catheter to obtain one or more images or a live video feed for reference and/or for navigation in a lumen of an object, target, or sample; (i) displaying the one or more images or the live video feed on a display; and/or (ii) keeping the one or more images or the live video feed displayed, via the host processor, on a display even where the error is detected to allow for operation, movement, and/or removable of the bendable robot or catheter.

In one or more methods, one or more of the following may occur or exist: (i) the bendable robot or catheter includes a distal bending section or portion, wherein the distal bending section or portion is commanded or instructed automatically or based on an input of a user of the bendable robot or catheter; (ii) the bendable robot or catheter includes a plurality of bending sections or portions including a distal or most distal bending portion or section and the rest of the plurality of the bending sections or portions, where the plurality of bending sections or portions each include driving wires that operate to bend a respective section or portion of the plurality of sections or portions, and where the driving wires are connected to the robot controlling apparatus or actuator or to respective one or more motors of the robot controlling apparatus or actuator so that the robot controlling apparatus or actuator operates to bend one or more of the plurality of bending sections or portions using the driving wires; and/or (iii) the robot controlling apparatus further comprises a slider, a rail, and/or a translational stage, and the one or more processors further operate to instruct or command the forward motion, or the motion in a set or predetermined direction, of the slider, the rail, the translational stage, and/or the bendable robot or catheter automatically or autonomously and/or based on an input of a user of the bendable robot or catheter.

In one or more embodiments, a non-transitory computer-readable storage medium may store at least one program for causing a computer to execute a method for performing error detection and/or error removal for a robot controlling apparatus/actuator and/or a bendable robot or catheter, the method comprising: checking a status of one or more communications of the robot controlling apparatus or actuator with a host processor of a host apparatus; determining whether an error is detected in the one or more communications with the host processor; and in a case where the error is detected in the one or more communications with the host processor, stopping a bendable robot or catheter from moving, bending, or changing a state of the bendable robot or catheter. One or more storage mediums may further include any combination of continuum robot and/or method feature(s) discussed herein.

In accordance with one or more embodiments of the present disclosure, apparatuses and systems, and methods and storage mediums for performing navigation, movement, and/or control, and/or for performing depth map-driven autonomous advancement of a multi-section continuum robot (e.g., in one or more airways, in one or more lungs, in one or more bronchoscopy pathways, in a lumen of a target/sample/object, in a vessel, etc.) and/or for performing or using one or more communication features may operate to characterize biological objects, such as, but not limited to, blood, mucus, lesions, tissue, etc.

Any discussion of a state, pose, position, orientation, navigation, path, or other state type discussed herein is discussed merely as a non-limiting, non-exhaustive embodiment example, and any state or states discussed herein may be used interchangeably/alternatively or additionally with the specifically mentioned type of state. Autonomous driving and/or control technique(s) may be employed to adjust, change, or control any state, pose, position, orientation, navigation, path, or other state type that may be used in one or more embodiments for a continuum robot or steerable catheter.

Physicians or other users of the apparatus or system may have reduced or saved labor and/or mental burden using the apparatus or system due to the navigation planning, autonomous navigation, control, and/or orientation (or pose, or position, etc.) feature(s) of the present disclosure. Additionally, one or more features of the present disclosure may achieve a minimized or reduced interaction with anatomy (e.g., of a patient), object, or target (e.g., tissue) during use, which may reduce the physical and/or mental burden on a patient or target. In one or more embodiments of the present disclosure, a labor of a user to control and/or navigate (e.g., rotate, translate, etc.) the imaging apparatus or system or a portion thereof (e.g., a catheter, a probe, a camera, one or more sections or portions of a catheter, probe, camera, etc.) is saved or reduced via use of the navigation planning, autonomous navigation, control, and/or communication/error communication feature(s).

One or more embodiments of the present disclosure may be used in clinical application(s), such as, but not limited to, intervascular imaging, intravascular imaging, bronchoscopy, atherosclerotic plaque assessment, cardiac stent evaluation, intracoronary imaging using blood clearing, balloon sinuplasty, sinus stenting, arthroscopy, ophthalmology, ear research, veterinary use and research, etc.

In accordance with at least another aspect of the present disclosure, one or more technique(s) discussed herein may be employed as or along with features to reduce the cost of at least one of manufacture and maintenance of the one or more apparatuses, devices, systems, and storage mediums by reducing or minimizing a number of optical and/or processing components and by virtue of the efficient techniques to cut down cost (e.g., physical labor, mental burden, fiscal cost, time and complexity, etc.) of use/manufacture of such apparatuses, devices, systems, and storage mediums.

The following paragraphs describe certain explanatory embodiments. Other embodiments may include alternatives, equivalents, modifications, and combinations of one or more features. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods, and one or more storage mediums using imaging, imaging adjustment or correction technique(s), communication/error communication technique(s), autonomous navigation and/or planning technique(s), and/or other technique(s) are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 illustrates at least one embodiment of an imaging, continuum robot, or endoscopic apparatus or system in accordance with one or more aspects of the present disclosure;

FIG. 2 is a schematic diagram showing at least one embodiment of an imaging, steerable catheter, or continuum robot apparatus or system in accordance with one or more aspects of the present disclosure;

FIGS. 3A-3B illustrate at least one embodiment example of a continuum robot and/or medical device that may be used with one or more technique(s), including autonomous driving/navigation and/or planning technique(s), in accordance with one or more aspects of the present disclosure;

FIGS. 3C-3D illustrate one or more principles of catheter or continuum robot tip manipulation by actuating one or more bending segments of a continuum robot or steerable catheter 104 of FIGS. 3A-3B in accordance with one or more aspects of the present disclosure;

FIG. 4 is a schematic diagram showing at least one embodiment of an imaging, continuum robot, steerable catheter, or endoscopic apparatus or system in accordance with one or more aspects of the present disclosure;

FIG. 5 is a schematic diagram showing at least one embodiment of a console or computer that may be used with one or more autonomous driving/navigation and/or planning technique(s) in accordance with one or more aspects of the present disclosure;

FIG. 6 is a flowchart of at least one embodiment of a method for planning an operation of at least one embodiment of a continuum robot or steerable catheter apparatus or system in accordance with one or more aspects of the present disclosure;

FIG. 7A illustrates at least one embodiment example of a system that operates to allow a user to guide and observe the movement of a medical device within a patient in accordance with one or more aspects of the present disclosure;

FIG. 7B illustrates diagrammatically at least one embodiment example of a steerable medical system in accordance with one or more aspects of the present disclosure;

FIG. 8A illustrates a lung with a pathway for continuum robot or endoscope insertion in accordance with one or more aspects of the present disclosure;

FIG. 8B is a diagram of at least one embodiment example of a bendable robot apparatus or system in accordance with one or more aspects of the present disclosure;

FIG. 9A is a diagram of at least one embodiment example of system level behaviors and/or functions in accordance with one or more aspects of the present disclosure;

FIG. 9B is a flowchart for at least at least one embodiment of high level system behaviors and/or functions in accordance with one or more aspects of the present disclosure;

FIG. 10 shows a schematic diagram of an embodiment of a computer that may be used with one or more embodiments of an apparatus or system, or one or more methods, discussed herein in accordance with one or more aspects of the present disclosure;

FIG. 11 shows a schematic diagram of another embodiment of a computer that may be used with one or more embodiments of an imaging apparatus or system, or methods, discussed herein in accordance with one or more aspects of the present disclosure;

FIG. 12 shows a schematic diagram of at least an embodiment of a system using a computer or processor, a memory, a database, and input and output devices in accordance with one or more aspects of the present disclosure;

FIG. 13 shows a created architecture of or for a regression model(s) that may be used for navigation planning, autonomous navigation, movement detection, and/or control techniques, host/robot communication techniques, and/or any other technique discussed herein in accordance with one or more aspects of the present disclosure;

FIG. 14 shows a convolutional neural network architecture that may be used for navigation planning, autonomous navigation, movement detection, and/or control techniques, host/robot communication techniques, and/or any other technique discussed herein in accordance with one or more aspects of the present disclosure;

FIG. 15 shows a created architecture of or for a regression model(s) that may be used for navigation planning, autonomous navigation, movement detection, and/or control techniques, host/robot communication techniques, and/or any other technique discussed herein in accordance with one or more aspects of the present disclosure; and

FIG. 16 is a schematic diagram of or for a segmentation model(s) that may be used for navigation planning, autonomous navigation, movement detection, and/or control techniques, host/robot communication techniques, and/or any other technique discussed herein in accordance with one or more aspects of the present disclosure.

### DETAILED DESCRIPTION OF THE PRESENT DISCLOSURE

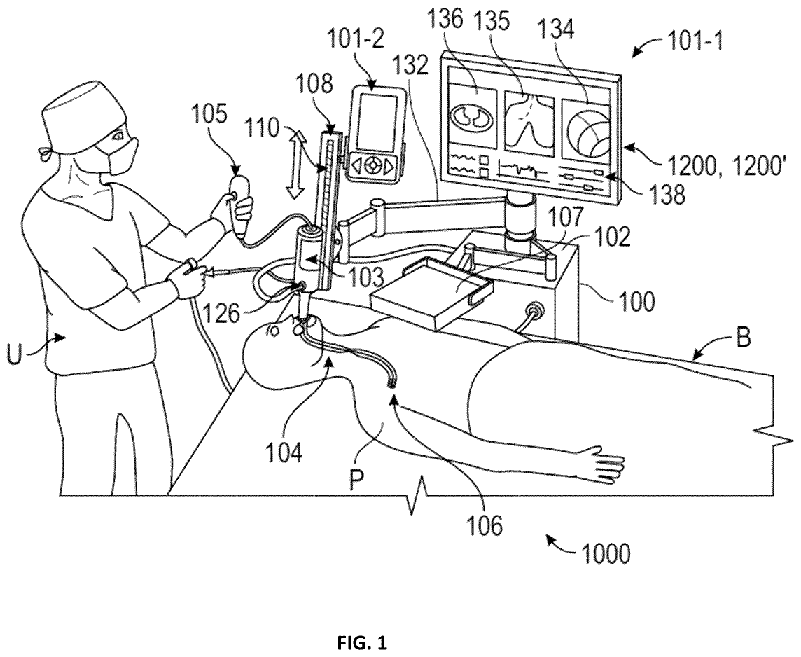

One or more devices, systems, methods and storage mediums for viewing, imaging, and/or characterizing tissue, or an object or sample, using one or more imaging techniques or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure, are described diagrammatically and visually in FIGS. 1 through 16.

One or more embodiments of the present disclosure avoid the aforementioned issues by providing a simple and fast method or methods that provide host and robot communication, error avoidance or elimination communication, navigation planning, autonomous driving/navigation, movement detection, and/or control technique(s) as discussed herein. In one or more embodiments of the present disclosure, host and robot communication, error avoidance or elimination communication, navigation planning, autonomous navigation, movement detection, and/or control techniques may be performed using artificial intelligence and/or one or more processors as discussed in the present disclosure. In one or more embodiments, host and robot communication, error avoidance or elimination communication, navigation planning, autonomous navigation, movement detection, and/or control is/are performed to reduce the amount of skill or training needed to perform imaging, medical imaging, one or more procedures (e.g., bronchoscopies), etc., and may reduce the time and cost of imaging or an overall procedure or procedures. In one or more embodiments, the host and robot communication, error avoidance or elimination communication, navigation planning, autonomous navigation, movement detection, and/or control techniques may be used with a co-registration (e.g., CT co-registration, cone-beam CT (CBCT) co-registration, etc.) to enhance a successful targeting rate for a predetermined sample, target, or object (e.g., a lung, a portion of a lung, a vessel, a nodule, etc.) by minimizing human error. CBCT may be used to locate a target, sample, or object (e.g., the lesion(s) or nodule(s) of a lung or airways) along with an imaging device (e.g., a steerable catheter, a continuum robot, etc.) and to co-register the target, sample, or object (e.g., the lesions or nodules) with the device shown in an image to achieve proper guidance.

Accordingly, it is a broad object of the present disclosure to provide imaging (e.g., computed tomography (CT), Magnetic Resonance Imaging (MRI), etc.) apparatuses, systems, methods, and storage mediums for using host and robot communication, error avoidance or elimination communication, navigation, and/or control method or methods (manual or automatic) in one or more apparatuses or systems (e.g., an imaging apparatus or system, an endoscopic imaging device or system, etc.). It is also a broad object of the present disclosure to provide imaging (e.g., computed tomography (CT), Magnetic Resonance Imaging (MRI), etc.) apparatuses, systems, methods, and storage mediums for using host and robot communication, error avoidance or elimination communication, navigation, and/or control method or methods for achieving navigation planning, autonomous navigation, movement detection, and/or control through a target, sample, or object (e.g., lung airway(s) during bronchoscopy) in one or more apparatuses or systems (e.g., an imaging apparatus or system, an endoscopic imaging device or system, etc.).

By applying the target detection method(s) of the present disclosure, movement of a robot may be automatically calculated and navigation planning, autonomous navigation, and/or control to a target, sample, or object (e.g., a nodule, a lung, an airway, a predetermined location in a sample, a predetermined location in a patient, etc.) may be achieved. Additionally, by applying several levels (such as, but not limited to, three or more levels) of target detection, the advancement, movement, and/or control of the robot may be secured in one or more embodiments (e.g., the robot will not fall into a loop). In one or more embodiments, automatically calculating the movement of the robot may be provided (e.g., targeting an airway during a bronchoscopy or other lung-related procedure/imaging may be performed automatically so that any next move or control is automatic), and navigation planning and/or autonomous navigation to a predetermined target, sample, or object (e.g., a nodule, a lung, a location in a sample, a location in a patient, etc.) is feasible and may be achieved (e.g., such that a CT path does not need to be extracted, any other pre-processing may be avoided or may not need to be extracted, etc.).

The navigation planning, autonomous navigation, movement detection, and/or control may be employed so that an apparatus or system may operate to: use a depth map produced by processing one or more images obtained by or coming from a continuum robot or steerable catheter (e.g., such as, but not limited to, a bronchoscopy robotic device or system (such as, but not limited to, a robotic catheter device(s), system(s), and method(s) discussed in PCT/US2023/062508, filed on Feb. 13, 2023, which is incorporated by reference herein in its entirety)); apply thresholding using an automated method; fit a one or more set or predetermined geometric shapes or one or more circles, rectangles, squares, ovals, octagons, and/or triangles/blob in or on one or more detected objects; set a center or portion of the one or more set or predetermined geometric shapes or one or more circles, rectangles, squares, ovals, octagons, and/or triangles as a target for a next movement of the continuum robot or steerable catheter; in a case where one or more targets are not detected, then apply peak detection to the depth map and use one or more detected peaks as the one or more targets; in a case where one or more peaks are not detected, then use a deepest point of the depth map as the one or more targets; and/or automatically advancing the continuum robot or steerable catheter to the detected one or more targets or choose automatically, semi-automatically, or manually one of the detected one or more targets. In one or more embodiments where a target position is outside an acceptable or predetermined area, then the method may further include returning to the depth map generation step. In one or more embodiments, automatic targeting the movement, navigation, and/or control may be provided (e.g., in a lung application, airway targeting which plans the next move may be automatic). In one or more embodiments, the one or more processors may apply thresholding to define an area of a target, sample, or object (e.g., to define an area of an airway/vessel or other target). In one or more embodiments, fitting the one or more set or predetermined geometric shapes or one or more circles, rectangles, squares, ovals, octagons, and/or triangles in the one or more detected objects may include blob detection and/or peak detection to identify the one or more targets and/or to confirm the identified or detected one or more targets. The one or more processors may further operate to: take a still image or images, use or process a depth map for the taken still image or images, apply thresholding to the taken still image or images and detect one or more objects, fit a one or more set or predetermined geometric shapes or one or more circles, rectangles, squares, ovals, octagons, and/or triangles/blob for the one or more objects of the taken still image or images, define one or more targets for a next movement of the continuum robot or steerable catheter based on the taken still image or images; and advance the continuum robot or steerable catheter to the one or more targets or choose automatically, semi-automatically, or manually one of the detected one or more targets. The one or more processors further operate to repeat any of the features (such as, but not limited to, obtaining a depth map, performing thresholding, performing a fit based on one or more set or predetermined geometric shapes or based on one or more circles, rectangles, squares, ovals, octagons, and/or triangles, performing peak detection, determining a deepest point, etc.) of the present disclosure for a next or subsequent image or images. Such next or subsequent images may be evaluated to distinguish from where to register the continuum robot or steerable catheter with an external image, and/or such next or subsequent images may be evaluated to perform registration or co-registration for changes due to movement, breathing, or any other change that may occur during imaging or a procedure with a continuum robot or steerable catheter. In one or more embodiments, a navigation plan may include (and may not be limited to) one or more of the following: a next movement of the continuum robot, one or more next movements of the continuum robot, one or more targets, all of the next movements of the continuum robot, all of the determined next movements of the continuum robot, one or more next movements of the continuum robot to reach the one or more targets, etc. In one or more embodiments, the navigation plan may be updated or data may be added to the navigation plan, where the data may include any additionally determined next movement of the continuum robot.

Any discussion of a state, pose, position, orientation, navigation, path, or other state type discussed herein is discussed merely as a non-limiting, non-exhaustive embodiment example, and any state or states discussed herein may be used interchangeably/alternatively or additionally with the specifically mentioned type of state. Host and robot communication, error avoidance or elimination communication, autonomous driving, and/or control technique(s) may be employed to adjust, change, or control any state, pose, position, orientation, navigation, path, or other state type that may be used in one or more embodiments for a continuum robot or steerable catheter.

Physicians or other users of the apparatus or system may have reduced or saved labor and/or mental burden using the apparatus or system due to the host and robot communication, error avoidance or elimination communication, navigation planning, autonomous navigation, control, and/or orientation (or pose, or position, etc.) feature(s) of the present disclosure. Additionally, one or more features of the present disclosure may achieve a minimized or reduced interaction with anatomy (e.g., of a patient), object, or target (e.g., tissue) during use, which may reduce the physical and/or mental burden on a patient or target. In one or more embodiments of the present disclosure, a labor of a user to control and/or navigate (e.g., rotate, translate, etc.) the imaging apparatus or system or a portion thereof (e.g., a catheter, a probe, a camera, one or more sections or portions of a catheter, probe, camera, etc.) is saved or reduced via use of the host and robot communication, error avoidance or elimination communication, navigation planning, autonomous navigation, and/or control.

An imaging device or system, or a portion of the imaging device or system (e.g., a catheter, a probe, etc.), the continuum robot, and/or the steerable catheter may include multiple sections or portions, and the multiple sections or portions may be multiple bending sections or portions. The imaging device or system may include host and robot communication, error avoidance or elimination communication, manual and/or automatic navigation, and/or control features. For example, a user of the imaging device or system (or steerable catheter, continuum robot, etc.) may control each section or portion, and/or the imaging device or system (or steerable catheter, continuum robot, etc.) may operate to automatically control (e.g., robotically control) each section or portion, such as, but not limited to, via one or more host and robot communication, error avoidance or elimination communication, navigation planning, autonomous navigation, movement detection, and/or control techniques.

Host and robot communication, error avoidance or elimination communication, navigation, control, and/or orientation feature(s) may include, but are not limited to, implementing mapping of a pose (angle value(s), plane value(s), etc.) of a first portion or section (e.g., a tip portion or section, a distal portion or section, a predetermined or set portion or section, a user selected or defined portion or section, etc.) to a stage position/state (or a position/state of another structure being used to map path or path-like information), controlling angular position(s) of one or more of the multiple portions or sections, controlling rotational orientation or position(s) of one or more of the multiple portions or sections, controlling (manually or automatically (e.g., robotically)) one or more other portions or sections of the imaging device or system (e.g., continuum robot, steerable catheter, etc.) to match the navigation/orientation/position/pose of the first portion or section in a case where the one or more other portions or sections reach (e.g., subsequently reach, reach at a different time, etc.) the same position (e.g., in a target, in an object, in a sample, in a patient, in a frame or image, etc.) during navigation in or along a first direction of a path of the imaging device or system, controlling each of the sections or portions of the imaging device or system to retrace and match prior respective position(s) of the sections or portions in a case where the imaging device or system is moving or navigated in a second direction (e.g., in an opposite direction along the path, in a return direction along the path, in a retraction direction along the path, etc.) along the path, etc. For example, an imaging device or system (or portion thereof, such as, but not limited to, a probe, a catheter, a camera, etc.) may enter a target along a path where a first section or portion of the imaging device or system (or portion of the device or system) is used to set the navigation or control path and position(s), and each subsequent section or portion of the imaging device or system (or portion of the device or system) is controlled to follow the first section or portion such that each subsequent section or portion matches the orientation and position of the first section or portion at each location along the path. During retraction, each section or portion of the imaging device or system is controlled to match the prior orientation and position (for each section or portion) for each of the locations along the path. As such, an imaging device or system (or catheter, probe, camera, etc. of the device or system) may enter and exit a target, an object, a specimen, a patient (e.g., a lung of a patient, an esophagus of a patient, another portion of a patient, another organ of a patient, a vessel of a patient, etc.), etc. along the same path and using the same orientation for entrance and exit to achieve an optimal navigation, orientation, and/or control path. The host and robot communication, error avoidance or elimination communication, navigation, control, and/or orientation feature(s) are not limited thereto, and one or more devices or systems of the present disclosure may include any other desired host and robot communication, error avoidance or elimination communication, navigation, control, and/or orientation specifications or details as desired for a given application or use. In one or more embodiments and while not limited thereto, the first portion or section may be a distal or tip portion or section of the imaging device or system. In one or more embodiments, the first portion or section may be any predetermined or set portion or section of the imaging device or system, and the first portion or section may be predetermined or set manually by a user of the imaging device or system or may be set automatically by the imaging device or system.

In one or more embodiments of the present disclosure (and while not limited to only this definition), a “change of orientation” may be defined in terms of direction and magnitude. For example, each interpolated step may have a same direction, and each interpolated step may have a larger magnitude as each step approaches a final orientation. Due to kinematics of one or more embodiments, any motion along a single direction may be the accumulation of a small motion in that direction. The small motion may have a unique or predetermined set of wire position changes to achieve the orientation change. Large or larger motion(s) in that direction may use a plurality of the small motions to achieve the large or larger motion(s). Dividing a large change into a series of multiple changes of the small or predetermined/set change may be used as one way to perform interpolation. Interpolation may be used in one or more embodiments to produce a desired or target motion, and at least one way to produce the desired or target motion may be to interpolate the change of wire positions.

Any of the features of the present disclosure may be used with artificial intelligence (AI) feature(s), including the AI features discussed herein. Using artificial intelligence, for example (but not limited to), deep/machine learning, residual learning, a computer vision task (keypoint or object detection and/or image segmentation), using a unique architecture structure of a model or models, using a unique training process, using input data preparation techniques, using input mapping to the model, using post-processing and interpretation of the output data, etc., one or more embodiments of the present disclosure may achieve a better or maximum success rate of host and robot communication, error avoidance or elimination communication, navigation planning, autonomous navigation, movement detection, and/or control without (or with less) user interactions, and may reduce processing time to perform host and robot communication, error avoidance or elimination communication, navigation planning, autonomous navigation, movement detection, and/or control techniques. One or more artificial intelligence structures may be used to perform host and robot communication, error avoidance or elimination communication, navigation planning, autonomous navigation, movement detection, and/or control techniques, such as, but not limited to, a neural net or network (e.g., the same neural net or network that operates to determine whether a video or image from an endoscope or other imaging device is working; the same neural net or network that has obtained or determined the depth map, etc.; an additional neural net or network, a convolutional network (e.g., a convolutional neural network (CNN) may operate to use a visual output of a camera (e.g., a bronchoscopic camera, a catheter camera, a detector, etc.) to automatically detect one or more airways, one or more objects, one or more areas of one or more airways or objects, etc.), recurrent network, another network discussed herein, etc.). In one or more embodiments, an apparatus for performing host and robot communication, error avoidance or elimination communication, navigation planning, autonomous navigation, movement detection, and/or control using artificial intelligence may include: a memory; and one or more processors in communication with the memory, the one or more processors operating to: using or processing a depth map produced by processing one or more images obtained by or coming from a continuum robot or steerable catheter (e.g., such as, but not limited to, a bronchoscopy robotic device or system (such as, but not limited to, a robotic catheter device(s), system(s), and method(s) discussed in PCT/US2023/062508, filed on Feb. 13, 2023, which is incorporated by reference herein in its entirety), obtained by a continuum robot or steerable catheter from the memory, obtained via one or more neural networks, etc.); applying thresholding using an automated method and detecting one or more objects; fitting one or more set or predetermined geometric shapes or one or more circles, rectangles, squares, ovals, octagons, and/or triangles in or on the one or more detected objects; defining one or more targets for a next movement of the continuum robot or steerable catheter; and advancing the continuum robot or steerable catheter to the one or more targets or choose automatically, semi-automatically, or manually one of the detected one or more targets. In one or more embodiments, defining the one or more targets may include setting a center or portion(s) of the one or more set or predetermined geometric shapes or the one or more circles, rectangles, squares, ovals, octagons, and/or triangles as one or more targets for a next movement of the continuum robot or steerable catheter. In one or more embodiments, the method may further include the following steps: in a case where the one or more targets are not detected, then applying peak detection to the depth map and using one or more detected peaks as the one or more targets; and/or in a case where one or more peaks are not detected, then using a deepest point of the depth map as the one or more targets. In one or more embodiments, the continuum robot or steerable catheter may be automatically advanced during the advancing step. In one or more embodiments, automatic targeting the movement, host and robot communication, error avoidance or elimination communication, navigation, and/or control may be provided (e.g., in a lung application, airway targeting which plans the next move may be automatic). One or more of the artificial intelligence features discussed herein that may be used in one or more embodiments of the present disclosure, includes but is not limited to, using one or more of deep learning, a computer vision task, keypoint detection, a unique architecture of a model or models, a unique training process or algorithm, a unique optimization process or algorithm, input data preparation techniques, input mapping to the model, pre-processing, post-processing, and/or interpretation of the output data as substantially described herein or as shown in any one of the accompanying drawings. Neural networks may include a computer system or systems. In one or more embodiments, a neural network may include or may comprise an input layer, one or more hidden layers of neurons or nodes, and an output layer. The input layer may be where the values are passed to the rest of the model. While not limited thereto, in one or more continuum robot or steerable catheter application(s), the input layer may be the place where the transformed navigation, movement, and/or control data may be passed to a model for evaluation. In one or more embodiments, the hidden layer(s) may be a series of layers that contain or include neurons or nodes that establish connections between the neurons or nodes in the other hidden layers. Through training, the values of each of the connections may be altered so that, due to the training, the system/systems will trigger when the expected pattern is detected. The output layer provides the result(s) of the model. In the case of the continuum robot or steerable catheter host and robot communication, error avoidance or elimination communication, navigation planning, autonomous navigation, movement detection, and/or control application(s), this may be a Boolean (true/false) value for detecting the one or more targets, for detecting the one or more objects, detecting the one or more peaks, detecting the deepest point, or any other calculation, detection, or process/technique discussed herein. One or more features discussed herein may be determined using a convolutional auto-encoder, Gaussian filters, Haralick features, and/or thickness or shape of the sample(s), target(s), or object(s). In one or more embodiments, and while not limited thereto, residual network(s), such as deep residual network(s), wide residual network(s), aggregated residual transformation network(s), other types of residual network(s) (e.g., ResNet, ResNeXt, etc.), any combination thereof, etc., may be used to perform AI feature(s).

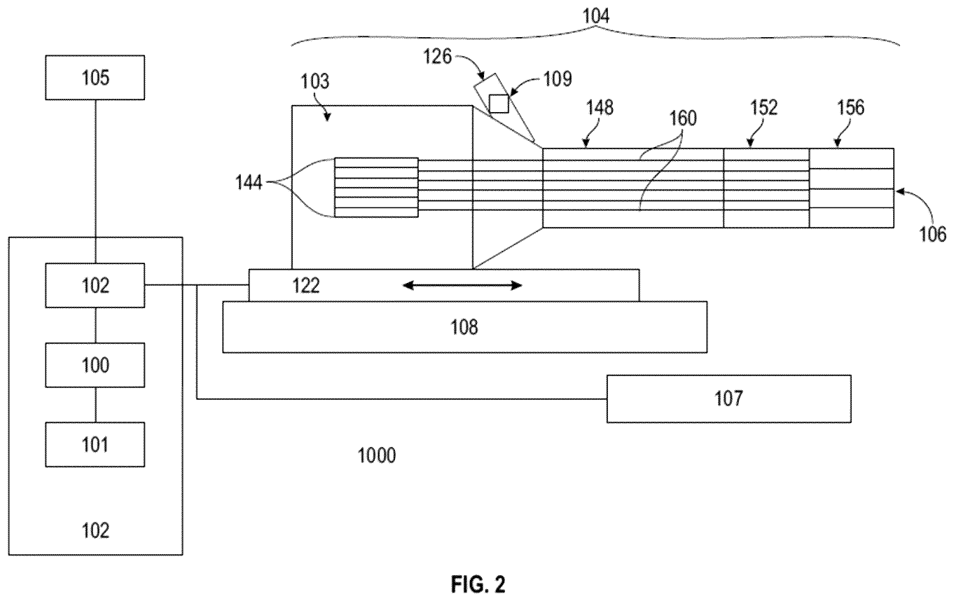

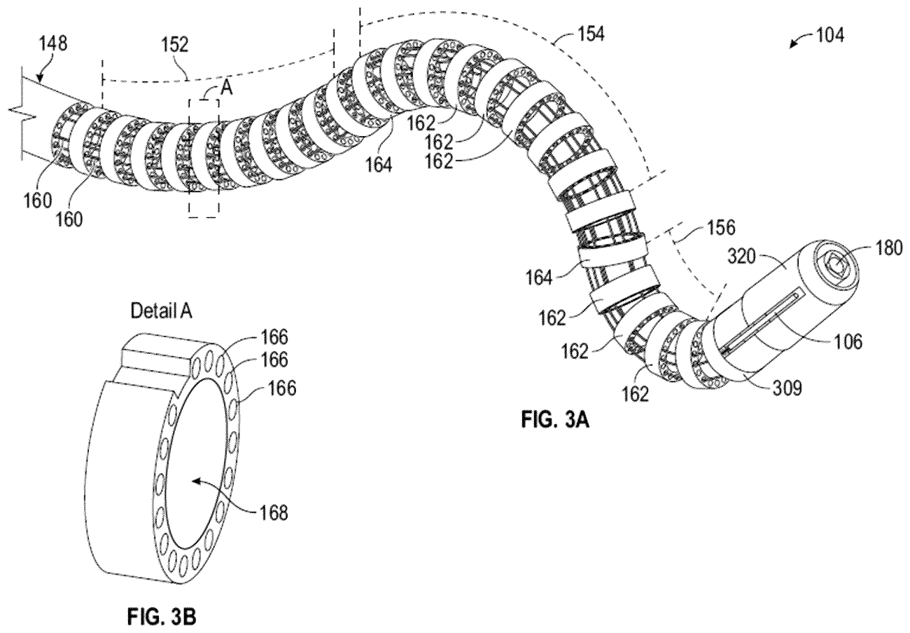

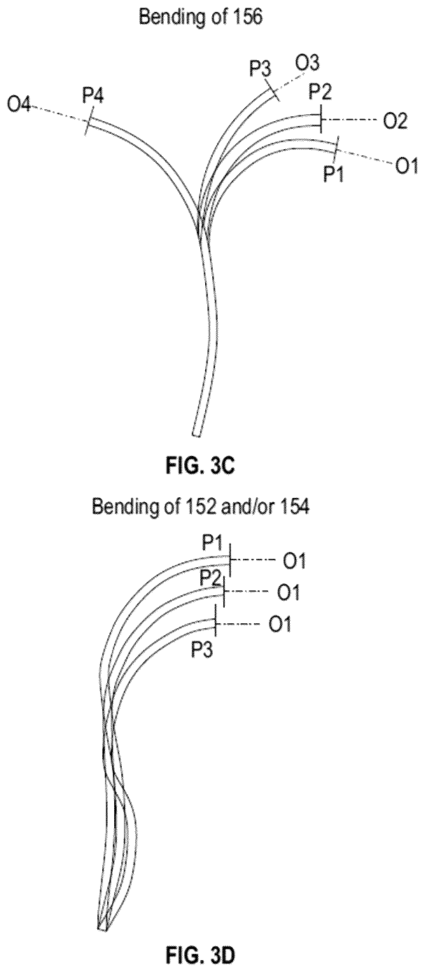

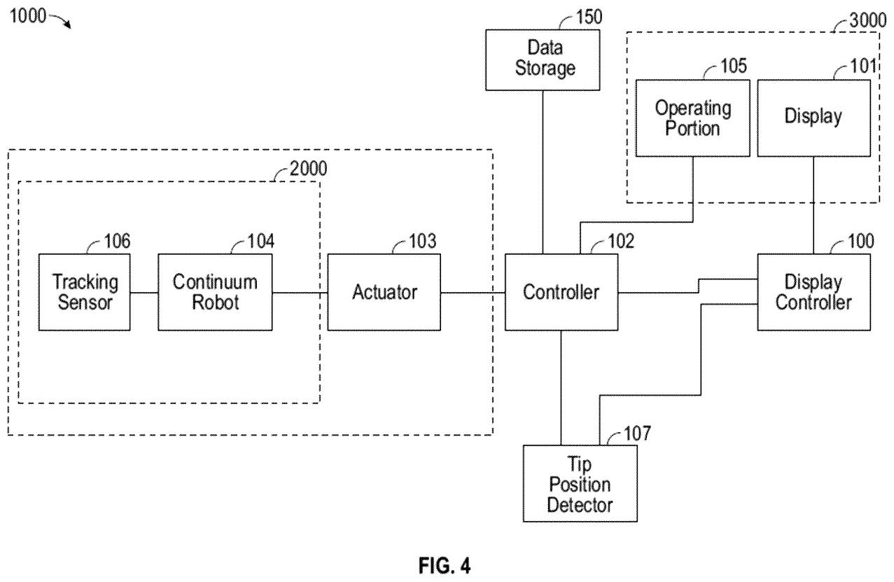

FIG. 1 illustrates a simplified representation of a medical environment, such as an operating room, where a robotic catheter apparatus/system 1000 may be used. FIG. 2 illustrates a functional block diagram that may be used in at least one embodiment of the robotic catheter apparatus/system 1000. FIGS. 3A-3D represent at least one embodiment of the catheter 104 (see FIGS. 3A-3B) and bending for the catheter 104 (as shown in FIGS. 3C-3D). FIG. 4 illustrates a logical block diagram that may be used for the robotic catheter apparatus/system 1000. In at least this embodiment example, the system 1000 may include a computer cart (see e.g., the controller 100, 102 in FIG. 1) operatively connected to a steerable catheter or continuum robot 104 via a robotic platform 108. The robotic platform 108 includes one or more than one robotic arm 132 and a rail 110 (see e.g., FIGS. 1-2) and/or linear translation stage 122 (see e.g., FIG. 2).

As shown in FIGS. 1-4 of the present disclosure, one or more embodiments of an apparatus/system 1000 for performing host and robot communication, error removal communication, navigation planning, autonomous navigation, movement detection, and/or control (e.g., for a continuum robot, a steerable catheter, etc.) may include one or more of the following: a display controller 100, a display 101-1, a display 101-2, a controller 102 (also referred to herein as an apparatus controller, a system controller, a processor, etc.), an actuator 103, a continuum device (also referred to herein as a “steerable catheter” or “an imaging device”) 104, an operating portion/input device 105, a camera 106 or a tracking sensor 106 (e.g., an electromagnetic (EM) tracking sensor), a catheter tip position/orientation/pose/state detector 107 (which may be optional (e.g., while a camera 106 or a camera 180 may be used without an EM tracking or tracking sensor in one or more embodiments, the tracking or EM tracking sensor may be used along with the camera 106 or the camera 180 in one or more embodiments; the position/state detector 107 may be used in one or more embodiments, but the position/state detector 107 may be optional in one or more embodiments such that the camera 106, 180 may be used without a position/state detector 107 in one or more embodiments, etc.), and a rail 110 (which may be attached to or combined with a linear translation stage 122) (for example, as shown in at least FIGS. 1 and 2). The apparatus/system 1000 may include one or more processors, such as, but not limited to, a display controller 100, a controller 102, a console or computer 1200 or 1200′, a CPU 120, a CPU 1201, any other processor or processors discussed herein, etc., that operate to execute a software program, to control the one or more adjustment, control, and/or smoothing technique(s) discussed herein, and to control display of a navigation screen on one or more displays 101. The one or more processors (e.g., the display controller 100, the controller 102, the CPU 120, the console or computer 1200 or 1200′, the CPU 1201, any other processor or processors discussed herein, etc.) may generate a three dimensional (3D) model of a structure (for example, a branching structure like airway of lungs of a patient, an object to be imaged, tissue to be imaged, etc.) based on images, such as, but not limited to, CT images, MRI images, etc. Alternatively, the 3D model may be received by the one or more processors (e.g., the display controller 100, the controller 102, the console or computer 1200 or 1200′, the CPU 1201, any other processor or processors discussed herein, etc.) from another device. A two-dimensional (2D) model may be used instead of 3D model in one or more embodiments. The 2D or 3D model may be generated before a navigation starts. Alternatively, the 2D or 3D model may be generated in real-time (in parallel with the navigation). In the one or more embodiments discussed herein, examples of generating a model of branching structure are explained. However, the models may not be limited to a model of branching structure. For example, a model of a route direct to a target may be used instead of the branching structure. Alternatively, a model of a broad space may be used, and the model may be a model of a place or a space where an observation or a work is performed by using a continuum robot 104 explained below.

In FIG. 1, a user U (e.g., a physician, a technician, etc.) may control the robotic catheter apparatus/system 1000 via a user interface unit (operation unit) to perform an intraluminal procedure on a patient P positioned on an operating table B. The user interface may include at least one of a main or first display 101-1 (a first user interface unit), a second display 101-2 (a second user interface unit), and a handheld controller/operating portion/input device 105 (a third user interface unit) (in one or more embodiments, an input device 1600 or any other input devices discussed herein may be used, such as, but not limited to, a mouse 1211, a keyboard 1210, a touch screen, etc.). The main or first display 101-1 may include, for example, a large display screen attached to the apparatus/system 1000 and/or the controllers 101, 102 of the apparatus/system 1000 or mounted on a wall of the operating room and may be, for example, designed as part of the robotic catheter apparatus/system 1000 or may be part of the operating room equipment. Optionally, there may be a secondary display 101-2 that is a compact (portable) display device configured to be removably attached to the robotic platform 108. Examples of the second or secondary display 101-2 may include, but are not limited to, a portable tablet computer, a mobile communication device (a cellphone), a tablet, a laptop, etc.

The steerable catheter 104 may be actuated via an actuator/actuator unit 103. The actuator unit 103 may be removably attached to the robotic platform 108 or any component thereof (e.g., the robotic arm 132, the rail 110, and/or the linear translation stage 122). The handheld controller/operating portion/input device 105 may include a gamepad-like controller with a joystick having shift levers and/or push buttons, and the controller/operating portion/input device 105 may be a one-handed controller or a two-handed controller. In one embodiment, the actuator unit 103 may be enclosed in a housing having a shape of a catheter handle. One or more access ports 126 may be provided in or around the catheter handle. The access port 126 may be used for inserting and/or withdrawing end effector tools and/or fluids when performing an interventional procedure of the patient P. In one or more embodiments, the operating portion/input device 105 may be used with or may include an input device 1600 or any other input devices discussed herein may be used, such as, but not limited to, a mouse 1211, a keyboard 1210, a touch screen, etc.

In one or more embodiments, the apparatus/system 1000 includes at least an apparatus/system controller 102, a display controller 100, and the main display 101-1. The main display 101-1 may include a conventional display device such as a liquid crystal display (LCD), an OLED display, a QLED display, etc. The main display 101-1 may provide or display a graphic interface unit (GUI) configured to display one or more views. These views may include a live view image 134, an intraoperative image 135, a preoperative image 136, and other procedural information 138. Other views that may be displayed include a model view, a navigational information view, and/or a composite view. The live image view 134 may be an image from a camera (e.g., the camera 106, the camera 180, etc.) at the tip of the catheter 104. The live image view 134 may also include, for example, information about the perception and navigation of the catheter 104. The preoperative image 136 may include pre-acquired 3D or 2D medical images of the patient P acquired by conventional imaging modalities such as computer tomography (CT), magnetic resonance imaging (MRI), ultrasound imaging, or any other desired imaging modality. The intraoperative image 135 may include images used for image guided procedure such images may be acquired by fluoroscopy or CT imaging modalities (or another desired imaging modality). The intraoperative image 135 may be augmented, combined, or correlated with information obtained from a sensor, camera image, or catheter data.

In the various embodiments where a catheter tip tracking sensor 106 is used in addition to the camera 106 and/or the camera 180, the sensor 106 may be located at the distal end of the catheter 104. The catheter tip tracking sensor 106 may be, for example, an electromagnetic (EM) sensor. If an EM sensor is used, a catheter tip position detector 107 may be included in the robotic catheter apparatus/system 1000; the catheter tip position detector 107 may include an EM field generator operatively connected to the apparatus/system controller 102. As aforementioned, the camera 106 may be used alone or in combination with the tracking sensor 106 and the position detector 107 to determine and output detected positional/state information to the apparatus/system controller 102. Suitable electromagnetic sensors for use with a steerable catheter may be used with any feature of the present disclosure, including the sensors discussed, for example, in U.S. Pat. No. 6,201,387 and in International Pat. Pub. WO 2020/194212 A1, which are incorporated by reference herein in their entireties.

While not limited to such a configuration, the display controller 100 may acquire position/orientation/navigation/pose/state (or other state) information of the continuum robot 104 from the controller 102. Alternatively, the display controller 100 may acquire the position/orientation/navigation/pose/state (or other state) information directly from the camera 106 and/or from a tip position/orientation/navigation/pose/state (or other state) detector 107. As aforementioned, the camera 106 may be used alone or in combination with the tracking sensor 106 and the position detector 107 to determine and output detected positional/state information to the apparatus/system controller 102. The continuum robot 104 may be a catheter device (e.g., a steerable catheter or probe device). The continuum robot 104 may be attachable/detachable to the actuator 103, and the continuum robot 104 may be disposable. While not limited thereto, the display controller 100, the controller 102, and/or any other processor or computer discussed herein may operate to perform the host and robot communication, error avoidance or elimination communication, the navigation, the planning, the imaging, and/or the control technique(s) or any other technique(s) discussed herein.

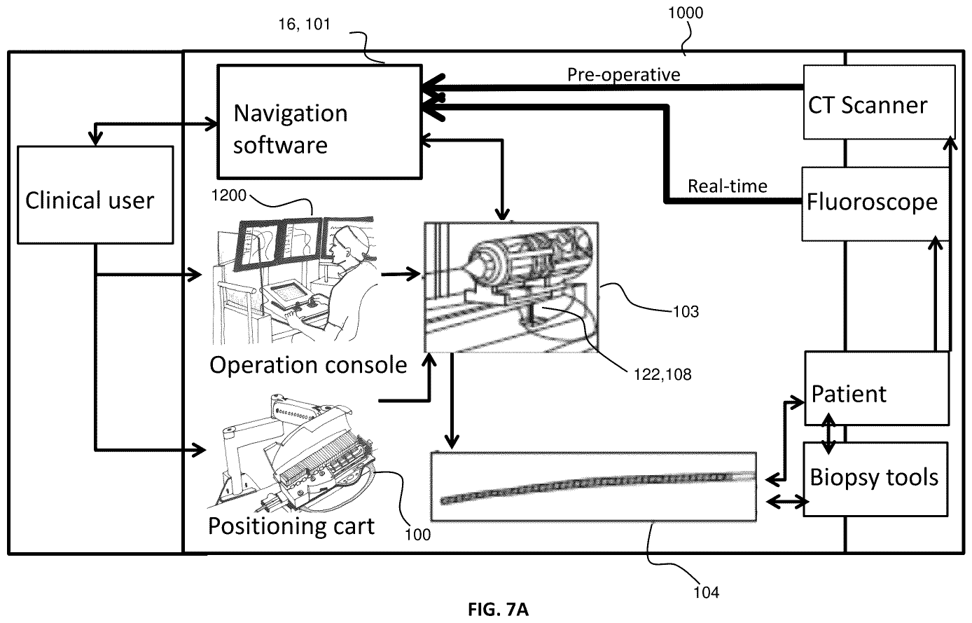

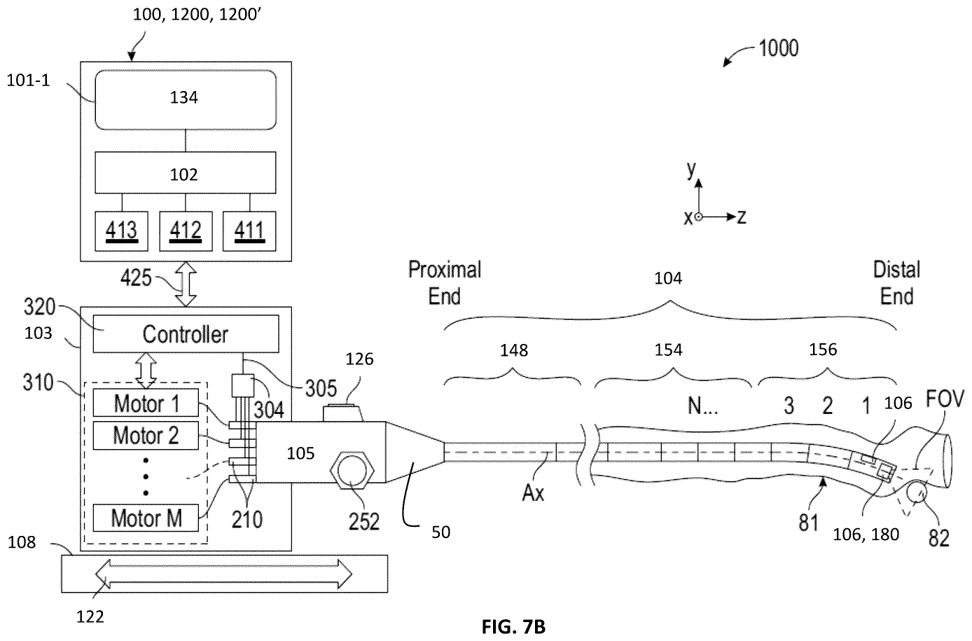

The robot-assisted endoscope apparatus/system 1000 may include a steerable instrument (a steerable medical device, which may include one or more of the bendable device 104, the operating portion/input device 105, one or more other components of the apparatus/system 1000, etc.) and/or the bendable device 104 operable by the user U (e.g., a physician, a technician, another operator, etc.) to perform an endoscopy procedure on the patient P. The robot-assisted endoscope apparatus/system 1000 may include a computer system 1200, 1200′ operatively attached to the steerable instrument and/or the bendable device 104 via a robotic arm 132 and/or a robotic platform 108. As shown in at least the embodiment example of FIG. 7B, the computer system 1200, 1200′ (e.g., a system console) includes a processor or central processing unit (CPU) 102 and/or any other CPU or processor discussed herein (e.g., the controller 100, the CPU 120, the CPU 1201, the processor or operation console 1200, the processor 1200′, etc.) and a display screen 101-1, such as a liquid crystal display (LCD), OLED or QLED display, and/or any other display screen discussed herein (e.g., the display 101-2, the display 1209, etc.). A storage memory 411 (e.g., ROM and RAM memory; a ROM memory (e.g., the ROM 110, the ROM 1202, etc.); a RAM memory (e.g., the RAM 130, the RAM 1203, etc.); data storage/HDD 150; the memory 1602; and/or any other memory discussed herein or known to those skilled in the art), a system interface 412 (e.g., one or more graphics processing units (“GPUs”; also called a visual processing unit (“VPU”)), one or more Field Programmable Gate Arrays (“FPGAs”), or other types of processing components (e.g., application specific integrated circuit(s) (ASIC)); an FPGA card; a CPU, a digital signal processor (“DSP”), FPGA, ASIC, or some other processing circuitry; any other processor or processing circuitry discussed herein; etc.), and a user interface 413 (e.g., mouse and keyboard; and/or any other user interface, mouse (e.g., mouse 1211 discussed below), keyboard (e.g., keyboard 1210 discussed below), touchscreen, or other user interface feature/structure discussed herein or known to those skilled in the art; etc.) may be operatively connected to the processor or CPU 102 and to the display screen 101-1.

In one or more embodiments, the steerable instrument and/or the bendable device 104 may include or be connected to a handle/operating portion/input device 105 and a bendable medical device 104 (e.g., a steerable sheath, a steerable catheter or probe, a steerable continuum robot, etc.), which may be removably connected to each other. The handle/operating portion/input device 105 may include or be connected to an actuator/actuator unit/actuator system 103 (e.g., which may include structure as shown in FIG. 2) which may receive electronic commands from a controller 100 (or any other processor discussed herein) and/or a computer system 1200, 1200′ to mechanically actuate the bendable medical device 104. The handle/operating portion/input device 105 may operate to be detachably mounted on the robotic platform 108 and/or the stage 122 (and/or the rail 110). The robotic platform 108 may include a robotic arm 132 and a stage 122 (and/or the rail 110) for robotically guiding the bendable medical device 104 towards a target site 82 within the subject or patient P. In one or more embodiments, the platform 108 may be disposed on a first surface of the stage 122 (as shown in FIG. 7B) or may be disposed on a second surface of the stage 122 (as shown in FIG. 2). When the handle/operating portion/input device 105 is not necessarily mounted on the robotic platform 108 and/or the stage 122 (and/or the rail 110), the handle/operating portion/input device 105 may be operated manually by the user U to control the bendable medical device 104 and/or the steerable instrument. For treating or examining the patient P, the steerable instrument may include one or more access ports 126 arranged on or around the handle/operating portion/input device 105 (see e.g., FIGS. 1 and 2). Access ports 126 may be used for inserting end effectors or for passing fluids to/from the patient P. In one or more embodiments as aforementioned, the camera 106 and/or the camera 180 may be positioned at the end of the bendable device 104. In one or more embodiments that may use the sensor 106 and the EM generator or detector 107, the electromagnetic (EM) field generator or detector 107 may interact with one or more EM sensors 106 arranged on the steerable sheath/catheter/probe and/or the bendable device 104 for tracking the position, shape, and/or orientation of the steerable sheath and/or the bendable device 104 while being inserted through a bodily lumen 81 (see e.g., as shown in FIG. 7B) towards a target site 82 within the patient P. The steerable instrument and/or the bendable device or medical device 104 may include a tool channel for a biopsy or other interventional tool. The clinical user may insert and retract/retreat the medical device and/or the bendable device 104 to perform, for example, a biopsy in the airways of the patient P.

During an endoscope procedure, the system processor or CPU 102 (or any other processor or CPU discussed herein) of computer system 100, 1200, 1200′ or any other computer system discussed herein operates to perform operations based on computer-executable code pre-stored in the system's memory 411 (see e.g., FIG. 7B) and/or any other memory discussed herein or known to those skilled in the art (such as, but not limited to, ROM and RAM memory; a ROM memory (e.g., the ROM 110, the ROM 1202, etc.); a RAM memory (e.g., the RAM 130, the RAM 1203, etc.); data storage/HDD 150; the memory 1602; etc.). As shown in FIG. 1, the display screen 101-1 may include a graphical user interface (GUI) configured to display one or more of patient information in an information window in the display screen 101-1, an endoscope live-image 134, an intra-operative image 135 (e.g., fluoroscopy), and a pre-operative image 136 (e.g., a slice image) of the patient P.

Similar to FIG. 1, FIG. 2 illustrates the robotic catheter apparatus/system 1000 including the apparatus/system controller 102 operatively connected to the display controller 100, which is connected to the first display 101-1 and to the second display 101-2. The apparatus/system controller 102 is also connected to the actuator 103 via the robotic platform 108 or any component thereof (e.g., the robotic arm 132, the rail 110, and/or the linear translation stage 122). The actuator unit 103 may include a plurality of motors 144 that operate to control a plurality of drive wires 160 (while not limited to any particular number of drive wires 160, FIG. 2 shows that six (6) drive wires 160 are being used in the subject embodiment example). The drive wires 160 travel through the steerable catheter or continuum robot 104. One or more access ports 126 may be located on the catheter 104 (and may include an insertion/extraction detector 109). The catheter 104 may include a proximal section 148 located between the actuator 103 and the proximal bending section 152, where the drive wires 160 operate to actuate the proximal bending section 152. Three of the six drive wires 160 continue through the distal bending section 156 where the drive wires 160 operate to actuate the distal bending section 156 and allow for a range of movement. FIG. 2 is shown with two bendable sections 152, 156. Other embodiments as described herein may have three bendable sections (see e.g., FIGS. 3A-3D). A single bending section may be provided, or alternatively, four or more bendable sections may be present in one or more embodiments of the catheter 104.

FIGS. 3A-3B show continuum robot 104 embodiment(s) that may be used in apparatus/system 1000 or other apparatus/system discussed herein. FIG. 3A shows embodiment(s) of a bendable portion and/or a steerable catheter 104. The steerable catheter 104 may have a non-steerable proximal section 148, a steerable distal section 156, and a catheter tip 320. The proximal section 148 and distal bendable section 156 (including portions 152, 154, and 156 in FIG. 3A) are joined to each other by a plurality of drive wires 160 arranged along the wall of the catheter 104. The proximal section 148 is configured with through-holes (or thru-holes) or grooves or conduits to pass drive wires 160 from the distal section 152, 154, 156 to the actuator unit 103. The distal section 152, 154, 156 is comprised of a plurality of bending segments including at least a distal segment 156, a middle segment 154, and a proximal segment 152. Each bending segment is bent by actuation of at least some of the plurality of drive wires 160 (driving members). The posture of the catheter 104 may be supported by supporting wires (support members) also arranged along the wall of the catheter 104 (as discussed in U.S. Pat. Pub. US2021/0308423, which is incorporated by reference herein in its entirety). The proximal ends of drive wires 160 are connected to individual actuators or motors 144 of the actuator unit 103, while the distal ends of the drive wires 160 are selectively anchored to anchor members in the different bending segments of the distal bendable section 152, 154, 156.

Each bending segment is formed by a plurality of ring-shaped components (rings) with through-holes (or thru-holes), grooves, or conduits along the wall of the rings. The ring-shaped components are defined as wire-guiding members 162 or anchor members 164 depending on a respective function(s) within the catheter 104. The anchor members 164 are ring-shaped components onto which the distal end of one or more drive wires 160 are attached in one or more embodiments. The wire-guiding members 162 are ring-shaped components through which some drive wires 160 slide through (without being attached thereto).

As shown in FIG. 3B, detail “A” obtained from the identified portion of FIG. 3A illustrates at least one embodiment of a ring-shaped component (a wire-guiding member 162 or an anchor member 164). Each ring-shaped component 162, 164 may include a central opening which may form a tool channel 168 and may include a plurality of conduits 166 (grooves, sub-channels, or through-holes (or thru-holes)) arranged lengthwise (and which may be equidistant from the central opening) along the annular wall of each ring-shaped component 162,164. Inside the ring-shaped component(s) 162, 164, an inner cover, such as is described in U.S. Pat. Pub. US2021/0369085 and US2022/0126060, which are incorporated by reference herein in their entireties, may be included to provide a smooth inner channel and to provide protection. The non-steerable proximal section 148 may be a flexible tubular shaft and may be made of extruded polymer material. The tubular shaft of the proximal section 148 also may have a central opening or tool channel 168 and plural conduits 166 along the wall of the shaft surrounding the tool channel 168. An outer sheath may cover the tubular shaft and the steerable section 152, 154, 156. In this manner, at least one tool channel 168 formed inside the steerable catheter 104 provides passage for an imaging device and/or end effector tools from the insertion port 126 to the distal end of the steerable catheter 104.

The actuator/actuator unit 103 may include, in one or more embodiments, one or more servo motors or piezoelectric actuators. The actuator unit 103 may operate to bend one or more of the bending segments of the catheter 104 by applying a pushing and/or pulling force to the drive wires 160.

As shown in FIG. 3A, each of the three bendable segments of the steerable catheter 104 has a plurality of drive wires 160. If each bendable segment is actuated by three drive wires 160, the steerable catheter 104 has nine driving wires arranged along the wall of the catheter 104. Each bendable segment of the catheter 104 is bent by the actuator unit 103 by pushing or pulling at least one of these nine drive wires 160. Force is applied to each individual drive wire in order to manipulate/steer the catheter 104 to a desired pose. The actuator unit 103 assembled with steerable catheter 104 may be mounted on the robotic platform 108 or any component thereof (e.g., the robotic arm 132, the rail 110, and/or the linear translation stage 122). The robotic platform 108, the rail 110, and/or the linear translation stage 122 may include a slider and a linear motor. In other words, the robotic platform 108 or any component thereof (e.g., the robotic arm 132, the rail 110, and/or the linear translation stage 122) is motorized, and may be controlled by the apparatus/system controller 102 to insert and remove the steerable catheter 104 to/from the target, sample, or object (e.g., the patient, the patient's bodily lumen, one or more airways, a lung, a vessel, a lumen, etc.).

An imaging device 180 that may be inserted through the tool channel 168 includes an endoscope camera (videoscope) along with illumination optics (e.g., optical fibers or LEDs) (or any other camera or imaging device, tool, etc. discussed herein or known to those skilled in the art). In one or more embodiments where a camera 106 is used, the imaging device 180 and/or the camera 106 may be used and inserted through the tool channel 168 for navigation, control, and/or imaging. The illumination optics provide light to irradiate the lumen and/or a lesion target which is a region of interest within the target, sample, or object (e.g., in a patient). End effector tools may refer to endoscopic surgical tools including clamps, graspers, scissors, staplers, ablation or biopsy needles, and other similar tools, which serve to manipulate body parts (organs or tumorous tissue) during imaging, examination, or surgery. The imaging device 180 may be what is commonly known as a chip-on-tip camera and may be color (e.g., take one or more color images) or black-and-white (e.g., take one or more black-and-white images). In one or more embodiments, a camera may support color and black-and-white images.

In some embodiments, the camera 106 and/or the camera 180 alone or in combination with a tracking sensor (e.g., an EM tracking sensor) 106 may be attached to the catheter tip 320. In an embodiment where an EM tracking sensor 106 is used along with the camera 106 (and/or the imaging device 180), the steerable catheter 104 and the tracking sensor 106 may be tracked by the tip position detector 107. Specifically, the tip position detector 107 detects a position of the tracking sensor 106, and outputs the detected positional information to the apparatus/system controller 102. Again, the camera 106 and/or the imaging device 180 may be used alone or in combination with the tracking sensor 106 and the position detector 107 to determine and output detected positional/state information to the apparatus/system controller 102 (and/or any other processor or controller discussed herein). The apparatus/system controller 102 (and/or any other processor or controller discussed herein) receives the positional information from the tip position detector 107 and continuously records and displays the position of the steerable catheter 104 with respect to the coordinate system of the target, sample, or object (e.g., a patient, a lung, an airway(s), a vessel, etc.). The apparatus/system controller 102 (and/or any other processor or controller discussed herein) operates to control the actuator unit 103 and the robotic platform 108 or any component thereof (e.g., the robotic arm 132, the rail 110, and/or the linear translation stage 122) in accordance with the manipulation commands input by the user U via one or more of the input and/or display devices (e.g., the handheld controller/operating portion/input device 105, a GUI at the main display 101-1, touchscreen buttons at the secondary display 101-2, etc.).

FIG. 3C and FIG. 3D show exemplary catheter tip manipulations by actuating one or more bending segments of the steerable catheter 104. As illustrated in FIG. 3C, manipulating only the most distal segment 156 of the steerable section may change the position and orientation of the catheter tip 320. On the other hand, manipulating one or more bending segments (152 or 154) other than the most distal segment may affect only the position of catheter tip 320, but may not affect the orientation of the catheter tip 320. In FIG. 3C, actuation of distal segment 156 changes the catheter tip from a position P1 having orientation O1, to a position P2 having orientation O2, to position P3 having orientation O3, to position P4 having orientation O4, etc. In FIG. 3D, actuation of the middle segment 152 and/or the middle segment 154 may change the position of the catheter tip 320 from a position P1 having orientation O1 to a position P2 and position P3 having the same orientation O1. Here, it should be appreciated by those skilled in the art that exemplary catheter tip manipulations shown in FIG. 3C and FIG. 3D may be performed during catheter navigation (e.g., while inserting the catheter 104 through tortuous anatomies, one or more targets, samples, objects, a patient, etc.). In the present disclosure, the one or more catheter tip manipulations shown in FIG. 3C and FIG. 3D may apply namely to the targeting mode applied after the catheter tip 320 has been navigated to a predetermined distance (a targeting distance) from the target, sample, or object.

The actuator 103 may proceed or retreat along the rail 110 and/or any other component of the platform (e.g., to translate the actuator 103, the continuum robot/catheter 104, etc.), and the actuator 103 and continuum robot 104 may proceed or retreat in and out of the patient's body or other target, object, or specimen (e.g., tissue, a lumen, etc.). As shown in FIG. 3B, the catheter device 104 may include a plurality of driving backbones and may include a plurality of passive sliding backbones. In one or more embodiments, the catheter device 104 may include at least nine (9) driving backbones and at least six (6) passive sliding backbones. The catheter device 104 may include an atraumatic tip at the end of the distal section of the catheter device 104.

FIG. 4 illustrates that an apparatus/1000 may include the apparatus/system controller or processor 102 which may operate to execute software programs and control the display controller or processor 100 to display a navigation screen (e.g., a live view image 134) on the main display 101-1 and/or the secondary display 101-2. The display controller 100 may include a graphics processing unit (GPU) or a video display controller (VDC) (or any other suitable hardware discussed herein or known to those skilled in the art).

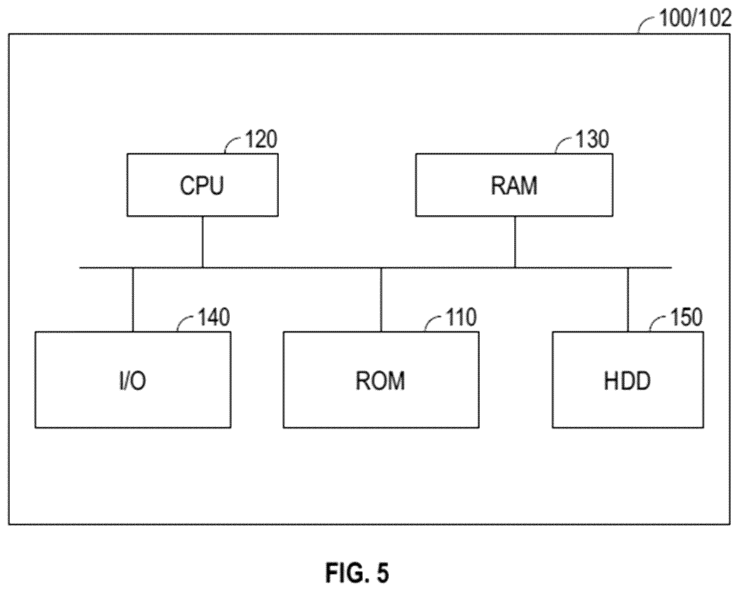

FIG. 5 illustrates components of the apparatus/system controller 102 and/or the display controller 100. The apparatus/system controller 102 and the display controller 100 may be configured separately. Alternatively, the apparatus/system controller 102 and the display controller 100 may be configured as one device. In either case, the apparatus/system controller 102 and the display controller 100 may include substantially the same components in one or more embodiments. For example, as shown in FIG. 5, the apparatus/system controller 102 and the display controller 100 may include a central processing unit (CPU 120) (which may be comprised of one or more processors (microprocessors)), a random access memory (RAM 130) module, an input/output (I/O 140) interface, a read only memory (ROM 110), and data storage memory (e.g., a hard disk drive (HDD 150) or solid state drive (SSD)). In one or more embodiments, any other RAM, ROM, storage memory, or other components discussed herein may be used for one or more processors or controllers, including at least controllers 100, 102.

In one or more embodiments, the ROM 110 and/or HDD 150 store the operating system (OS) software, and software programs necessary for executing the functions of the robotic catheter apparatus/system 1000 (or any other apparatus/system discussed herein, etc.) as a whole. The RAM 130 is used as a workspace memory. The CPU 120 executes the software programs developed in the RAM 130. The I/O 140 inputs, for example, positional information to the display controller 100 and/or the apparatus/system controller 102, and outputs information for displaying the navigation screen to the one or more displays (main display 101-1 and/or secondary display 101-2). In the embodiments described below, the navigation screen is a graphical user interface (GUI) generated by a software program but, it may also be generated by firmware, or a combination of software and firmware.

The apparatus/system controller 102 may control the steerable catheter 104 based on any known kinematic algorithms applicable to continuum or snake-like catheter robots. For example, the system controller controls the steerable catheter 104 based on an algorithm known as follow the leader (FTL) algorithm. With the FTL algorithm, the most distal segment 156 is actively controlled with forward kinematic values, while the middle segment 154 and the other middle or proximal segment 152 (following sections) of the steerable catheter 104 move at a first position in the same way as the distal section moved at the first position or a second position near the first position.

The display controller 100 may acquire position information of the steerable catheter 104 from apparatus/system controller 102. Alternatively, the display controller 100 may acquire the position information directly from the tip position detector 107. The steerable catheter 104 may be a single-use or limited-use catheter device. In other words, the steerable catheter 104 may be attachable to, and detachable from, the actuator unit 103 to be disposable.

During a procedure, the display controller 100 may generate and output a live-view image or a navigation screen to the main display 101-1 and/or the secondary display 101-2 based on the 3D model of a target, sample, or object (e.g., a lung, an airway, a vessel, a patient's anatomy (a branching structure), etc.) and the position information of at least a portion of the catheter (e.g., position of the catheter tip 320) by executing pre-programmed software routines. The navigation screen may indicate a current position of at least the catheter tip 320 on the 3D model. By observing the navigation screen, a user may recognize the current position of the steerable catheter 104 in the branching structure. Upon completing navigation to a desired target, one or more end effector tools may be inserted through the access port 126 at the proximal end of the catheter 104, and such tools may be guided through the tool channel 168 of the catheter body to perform an intraluminal procedure from the distal end of the catheter 104.

The tool may be a medical tool such as an endoscope camera, forceps, a needle, or other biopsy or ablation tools. The tool may be described as an operation tool or working tool. The working tool is inserted or removed through the working tool access port 126. In the embodiments below, at least one embodiment of using a steerable catheter 104 to guide a tool to a target is explained. The tool may include an endoscope camera or an end effector tool, which may be guided through a steerable catheter under the same principles. In a procedure there is usually a planning procedure, a registration procedure, a targeting procedure, and an operation procedure.

The one or more processors, such as, but not limited to, the display controller 100, the CPU 1201, etc., may generate and output a navigation screen to the one or more displays 101-1, 101-2 based on the 2D/3D model and the position/orientation/navigation/pose/state (or other state) information by executing the software. The navigation screen may indicate a current position/orientation/navigation/pose/state (or other state) of the continuum robot 104 on the 2D/3D model. By using the navigation screen, a user may recognize the current position/orientation/navigation/pose/state (or other state) of the continuum robot 104 in the branching structure. Any feature herein may be used with any navigation/pose/state feature(s) or other feature(s) discussed in International Patent Application No. PCT/US2024/031766, filed May 30, 2024, the disclosure of which is incorporated by reference herein in its entirety.

Any feature of the present disclosure may be used with any navigation/pose/state feature(s) and/or other feature(s) discussed in: International Patent Application No. PCT/US2024/037935, filed Jul. 12, 2024, the disclosure of which is incorporated by reference herein in its entirety; International Patent Application No. PCT/US2024/037924, the disclosure of which is incorporated by reference herein in its entirety; and International Patent Application No. PCT/US2024/037930, the disclosure of which is incorporated by reference herein in its entirety.

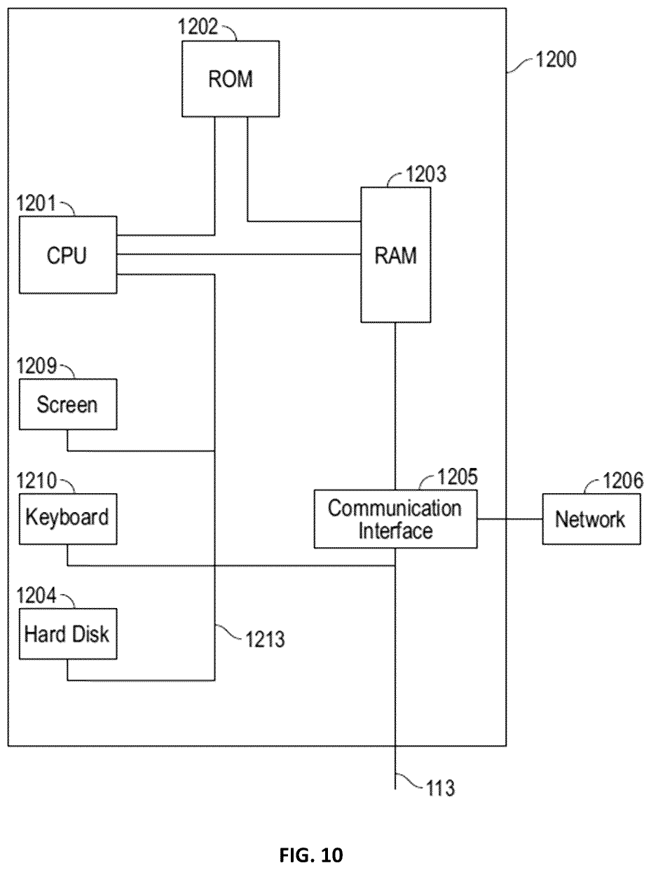

In one or more embodiments, the one or more processors, such as, but not limited to, the display controller 100 and/or the controller 102, may include, as shown in FIG. 5, at least one storage Read Only Memory (ROM) 110, at least one central processing unit (CPU) 120, at least one Random Access Memory (RAM) 130, at least one input and output (I/O) interface 140 and at least one Hard Disc Drive (HDD) 150 (see e.g., also data storage 150 of FIG. 4). A Solid State Drive (SSD) may be used instead of HDD 150 as the data storage 150. In one or more additional embodiments, the one or more processors, and/or the display controller 100 and/or the controller 102, may include structure as shown in any of FIGS. 10-16 as further discussed below.

The ROM 110 and/or HDD 150 operate to store software in one or more embodiments. The RAM 130 may be used as a work memory. The CPU 120 may execute the software program developed in the RAM 130. The I/O 140 operates to input the positional (or other state) information to the display controller 100 (and/or any other processor discussed herein) and to output information for displaying the navigation screen to the one or more displays 101-1, 101-2. In the embodiments below, the navigation screen may be generated by the software program. In one or more other embodiments, the navigation screen may be generated by a firmware.

One or more devices or systems, such as the apparatus/system 1000, may include a tip position/orientation/navigation/pose/state (or other state) detector 107 that operates to detect a position/orientation/navigation/pose/state (or other state) of the EM tracking sensor 106 and to output the detected positional (and/or other state) information to the controller 100 or 102 (e.g., as shown in FIGS. 1-2), or to any other processor(s) discussed herein.

The controller 102 may operate to receive the positional (or other state) information of the tip of the continuum robot 104 from the camera 106 and/or from the tip position/orientation/navigation/pose/state (or any other state discussed herein) detector 107. In one or more embodiments, the detector 107 may be optional such that the camera 106 may be used alone (e.g., without the tracking sensor 106 and the detector 107). The controller 100 and/or the controller 102 operates to control the actuator 103 in accordance with the manipulation by a user (e.g., manually), and/or automatically (e.g., by a method or methods run by one or more processors using software, by the one or more processors, using automatic manipulation in combination with one or more manual manipulations or adjustments, etc.) via one or more operation/operating portions or operational controllers/input device(s) 105 (e.g., such as, but not limited to a joystick as shown in FIGS. 1-2; see also, diagram of FIG. 4; see also, the mouse 1211, the keyboard 1210, the touch screen(s), or any other input devices discussed herein or known to those skilled in the art, which may be used as an input device(s) 105). The one or more displays 101-1, 101-2 and/or operation portion or operational controllers/operating portions/input devices 105 may be used as a user interface 3000 (also referred to as a receiving device) (e.g., as shown diagrammatically in FIG. 4). In an embodiment shown in FIGS. 1-2 or the embodiment shown in FIG. 4, the system(s) 1000 may include, as an operation unit, the display 101-1 (e.g., such as, but not limited to, a large screen user interface with a touch panel, first user interface unit, etc.), the display 101-2 (e.g., such as, but not limited to, a compact user interface with a touch panel, a second user interface unit, etc.) and the operating portion/input device 105 (e.g., such as, but not limited to, a joystick shaped user interface unit having shift lever/button, a third user interface unit, a gamepad, or other input device, etc.).

The controller 100 and/or the controller 102 (and/or any other processor discussed herein) may control the continuum robot 104 based on an algorithm known as follow the leader (IFTL) algorithm. The IFTL algorithm may be used in addition to the host and robot communication, error elimination and/or avoidance communication, navigation planning, and/or autonomous navigation features of the present disclosure. For example, by applying the FTL algorithm, the middle section and the proximal section (following sections) of the continuum robot 104 may move at a first position (or other state) in the same or similar way as the distal section moved at the first position (or other state) or a second position (or state) near the first position (or state) (e.g., during insertion of the continuum robot/catheter 104, by using the host and robot communication, error elimination and/or avoidance communication, navigation planning, autonomous navigation, movement, and/or control feature(s) of the present disclosure, etc.). Similarly, the middle section and the distal section of the continuum robot 104 may move at a first position or state in the same/similar/approximately similar way as the proximal section moved at the first position or state or a second position or state near the first position (e.g., during removal of the continuum robot/catheter 104). Additionally or alternatively, the continuum robot/catheter 104 may be removed by automatically and/or manually moving along the same or similar, or approximately same or similar, path that the continuum robot/catheter 104 used to enter a target (e.g., a body of a patient, an object, a specimen (e.g., tissue), etc.) using the IFTL algorithm, including, but not limited to, using FTL with the one or more host and robot communication, error elimination and/or avoidance communication, adjustment, correction, state, navigation, control, and/or smoothing technique(s) discussed herein.

Any of the one or more processors, such as, but not limited to, the controller 102 and the display controller 100, may be configured separately. As aforementioned, the controller 102 may similarly include a CPU 120, a RAM 130, an I/O 140, a ROM 110, and a HDD 150 as shown diagrammatically in FIG. 5. Alternatively, any of the one or more processors, such as, but not limited to, the controller 102 and the display controller 100, may be configured as one device (for example, the structural attributes of the controller 100 and the controller 102 may be combined into one controller or processor, such as, but not limited to, the one or more other processors discussed herein (e.g., computer, console, or processor 1200, 1200′, CPU 1201, GUI 1215, etc.)).

The apparatus/system 1000 may include a tool channel 126 for a camera, biopsy tools, or other types of medical tools (as shown in FIGS. 1-2). For example, the tool may be a medical tool, such as an endoscope, a forceps, a needle or other biopsy tools, etc. In one or more embodiments, the tool may be described as an operation tool or working tool. The working tool may be inserted or removed through a working tool insertion slot 126 (as shown in FIGS. 1-2). Any of the features of the present disclosure may be used in combination with any of the features, including, but not limited to, the tool insertion slot, as discussed in U.S. Pat. Pub. No. 2024/0112407 A1, published Apr. 4, 2024, the disclosure of which is incorporated by reference herein in its entirety.

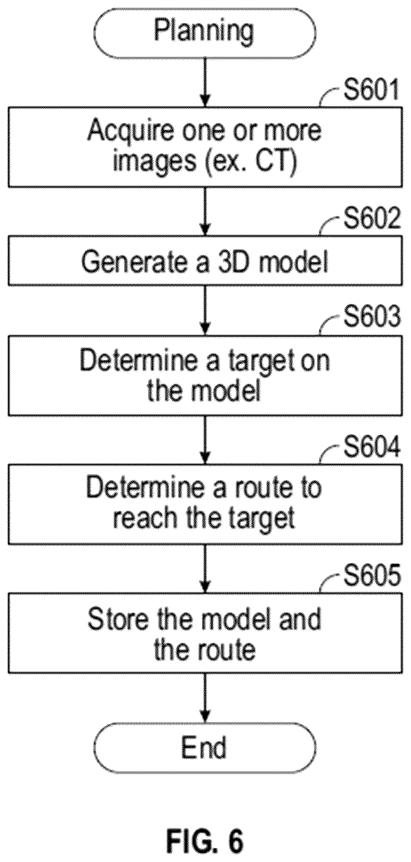

One or more of the features discussed herein may be used for planning procedures, including using one or more models for artificial intelligence applications. As an example of one or more embodiments, FIG. 6 is a flowchart showing steps of at least one planning procedure of an operation of the continuum robot/catheter device 104. One or more of the processors discussed herein may execute the steps shown in FIG. 6, and these steps may be performed by executing a software program read from a storage medium, including, but not limited to, the ROM 110 or HDD 150, by CPU 120 or by any other processor discussed herein. One or more methods of planning using the continuum robot/catheter device 104 may include one or more of the following steps: (i) In step s601, one or more images such, as CT or MRI images, may be acquired; (ii) In step s602, a three dimensional model of a branching structure (for example, an airway model of lungs or a model of an object, specimen or other portion of a body) may be generated based on the acquired one or more images; (iii) In step s603, a target on the branching structure may be determined (e.g., based on a user instruction, based on preset or stored information, etc.); (iv) In step s604, a route of the continuum robot/catheter device 104 to reach the target (e.g., on the branching structure) may be determined (e.g., based on a user instruction, based on preset or stored information, based on a combination of user instruction and stored or preset information, etc.); (v) In step s605, the generated model (e.g., the generated two-dimensional or three-dimensional model) and the decided route on the model may be stored (e.g., in the RAM 130 or HDD or data storage 150, in any other storage medium discussed herein, in any other storage medium known to those skilled in the art, etc.). In this way, a model (e.g., a 2D or 3D model) of a branching structure may be generated, and a target and a route on the model may be determined and stored before the operation of the continuum robot 104 is started.

Embodiments of using a catheter device/continuum robot 104 are explained, such as, but not limited to features for performing host and robot communication, error elimination and/or avoidance communication, navigation planning, autonomous driving/navigation, movement detection, and/or control technique(s).

The apparatus/system controller 102 (or any other controller, processor, computer, etc. discussed herein) may operate to perform a host and robot communication mode, an error elimination and/or avoidance communication mode, a navigation planning mode, and/or an autonomous navigation mode. For example, during the navigation planning mode and/or the autonomous navigation mode, the user does not need to control the bending and translational insertion position of the steerable catheter 104. The navigation planning and/or autonomous navigation mode may include or comprise: (1) a perception step, (2) a planning step, and (3) a control step. In the perception step, the apparatus/system controller 102 may receive an endoscope view (or imaging data) and may analyze the endoscope view (or imaging data) to find addressable airways from the current position/orientation of the steerable catheter 104. At an end of this analysis, the apparatus/system controller 102 identifies or perceives these addressable airways as paths in the endoscope view (or imaging data).

The planning step is a step to determine a target path, which is the destination for the steerable catheter 104. While there are a couple of different approaches to select one of the paths as the target path, the present disclosure uniquely includes means to reflect user instructions concurrently for the decision of a target path among the identified or perceived paths. Once the apparatus/system 1000 determines the target paths while considering concurrent user instructions, the target path is sent to the next step, i.e., the control step.

The control step is a step to control the steerable catheter 104 and the linear translation stage 122 (or any other portion of the robotic platform 108) to navigate the steerable catheter 104 to the target path, pose, state, etc. This step may also be performed as an automatic step. The apparatus/system controller 102 operates to use information relating to the real time endoscope view (e.g., the view 134), the target path, and an internal design & status information on the robotic catheter apparatus/system 1000 (or any other apparatus/system discussed herein, etc.).

Through these three steps, the robotic catheter apparatus/system 1000 (or any other apparatus/system discussed herein, etc.) may navigate the steerable catheter 104 autonomously, which achieves reflecting the user's intention efficiently.

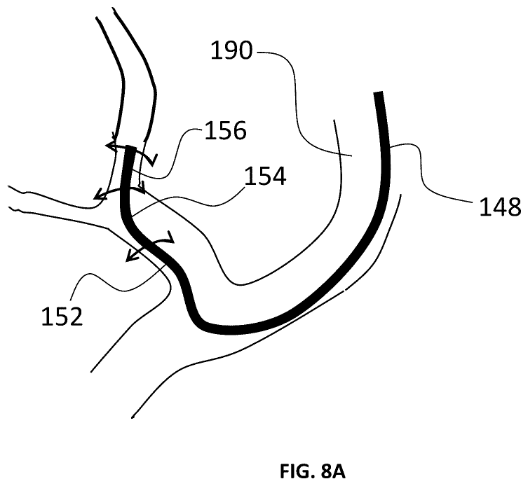

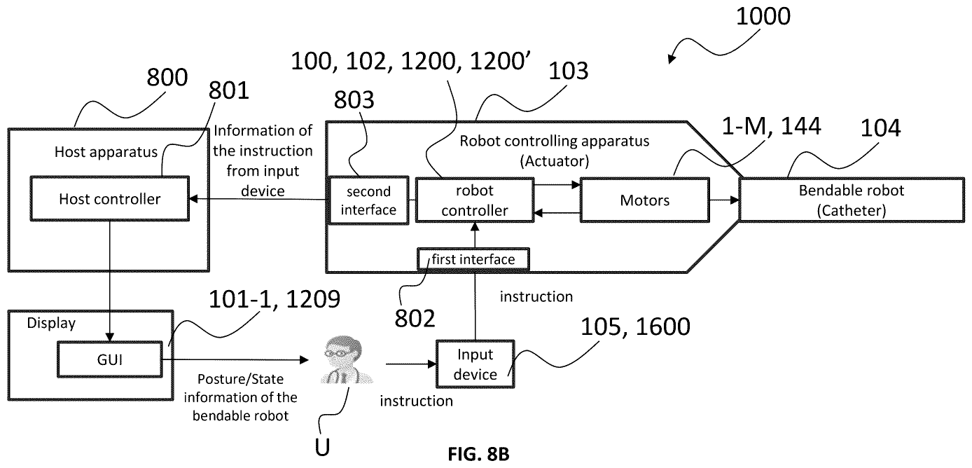

As shown in FIG. 1, the real-time endoscope view 134 may be displayed in a main display 101-1 (as a user input/output device) in the apparatus/system 1000 (or any other apparatus/system discussed herein, etc.). The user may see the airways in the real-time endoscope view 134 through the main display 101-1. This real-time endoscope view 134 may also be sent to the apparatus/system controller 102. In the perception step, the apparatus/system controller 102 may process the real-time endoscope view 134 and may identify path candidates by using image processing algorithms. Among these path candidates, the apparatus/system controller 102 may select the paths with the designed computation processes, and then may display the paths with a circle, octagon, or other geometric shape (e.g., one or more set or predetermined geometric shapes or one or more circles, rectangles, squares, ovals, octagons, triangles, any other shape discussed herein or known to those skilled in the art, any closed shape discussed herein or known to those skilled in the art, etc.) with the real-time endoscope view 134 (one example may be, as discussed further below, as shown in FIG. 8A).

In the planning step, the apparatus/system controller 102 may provide a cursor so that the user may indicate the target path by moving the cursor with the joystick or any other input device(s) 105, 1600 discussed herein. When the cursor is disposed or is located within the area of the path, the apparatus/system controller 102 operates to recognize the path with the cursor as the target path.

In a further embodiment example, the apparatus/system controller 102 may pause the motion of the actuator unit 103 and the linear translation stage 122 while the user is moving the cursor so that the user may select the target path with a minimal change of the real-time endoscope view 134 and paths since the apparatus/system 1000 (or any other apparatus/system discussed herein, etc.) would not move in such a scenario. Additionally or alternatively, the features of the present disclosure may be performed using artificial intelligence, including, but not limited to, the autonomous driving mode, the host and robot communication mode, the error avoidance and/or removal mode, the navigation mode, the planning mode, the imaging mode, the control mode, etc. For example, deep learning may be used for performing autonomous driving using deep learning for localization. Any features of the present disclosure may be used with artificial intelligence features discussed in J. Sganga, D. Eng, C. Graetzel, and D. B. Camarillo, “Autonomous Driving in the Lung using Deep Learning for Localization,” July 2019, Accessed: Jun. 28, 2023. [Online]. Available: https://arxiv.org/abs/1907.08136v1, the disclosure of which is incorporated by reference herein in its entirety.

In one or more embodiments, the apparatus/system controller 102 (or any other controller, processor, computer, etc. discussed herein) may operate to perform a depth map mode. A depth map may be generated or obtained from one or more images (e.g., bronchoscopic images, CT images, images of another imaging modality, etc.). A depth of each image may be identified or evaluated to generate the depth map or maps. The generated depth map or maps may be used to perform host and robot communication, error avoidance or removal communication, navigation planning, autonomous navigation, movement detection, and/or control of a continuum robot, a steerable catheter, an imaging device or system, etc. as discussed herein. In one or more embodiments, thresholding may be applied to the generated depth map or maps, or to the depth map mode, to evaluate accuracy for navigation purposes. For example, while not limited to only this type of a threshold, a threshold may be set for an acceptable distance between the ground truth (and/or a target camera location, a predetermined camera location, an actual camera location, etc.) and an estimated camera location for a catheter or continuum robot (e.g., the catheter or continuum robot 104). By way of a further example, the threshold may defined such that the distance between the ground truth (and/or a target camera location, a predetermined camera location, an actual camera location, etc.) and an estimated camera location is equal to or less than, or less than, a set or predetermined distance of one or more of the following: 5 mm, 10 mm, about 5 mm, about 10 mm, any other distance set by a user of the device (depending on a particular application). In one or more embodiments, the predetermined distance may be less than 5 mm or less than about 5 mm. Any other type of thresholding may be applied to the depth mapping to improve and/or confirm the accuracy of the depth map(s).

Additionally or alternatively, thresholding may be applied to segment the one or more images to help identify or find one or more objects and to ultimately help define one or more targets used for the host and robot communication, the error avoidance or removal communication, navigation planning, autonomous navigation, movement detection, and/or control features of the present disclosure. For example, a depth map or maps may be created or generated using one or more images (e.g., CT images, bronchoscopic images, images of another imaging modality, vessel images, etc.), and then, by applying a threshold to the depth map, the objects in the one or more images may be segmented (e.g., a lung may be segmented, one or more airways may be segmented, etc.). In one or more embodiments, the segmented portions of the one or more images (e.g., the one or more segmented airways, the segmented portions of a lung, a vessel, a lumen, etc.) may define one or more navigation targets for a next host and robot communication, error avoidance or removal communication, automatic robotic movement, navigation, and/or control. Examples of segmented airways and using thresholding (such as, but not limited to, watershed method(s), threshold selection method(s), peak detection method(s), using depth map(s), geometric shape/blob detection(s), etc.) are discussed in U.S. Prov. Pat. App. No. 63/742,346, filed on Jan. 6, 2025, which is incorporated by reference herein in its entirety.

The depth map(s) may be obtained, and/or the quality of the obtained depth map(s) may be evaluated, using artificial intelligence structure, such as, but not limited, convolutional neural networks, generative adversarial networks (GANs), neural networks, any other AI structure or feature(s) discussed herein, any other AI network structure(s) known to those skilled in the art, etc. For example, a generator of a generative adversarial network may operate to generate an image(s) that is/are so similar to ground truth image(s) that a discriminator of the generative adversarial network is not able to distinguish between the generated image(s) and the ground truth image(s). The generative adversarial network may include one or more generators and one or more discriminators. Each generator of the generative adversarial network may operate to estimate depth of each image (e.g., a CT image, a bronchoscopic image, etc.), and each discriminator of the generative adversarial network may operate to determine whether the estimated depth of each image (e.g., a CT image, a bronchoscopic image, etc.) is estimated (or fake) or ground truth (or real). In one or more embodiments, an AI network, such as, but not limited to, a GAN or a consistent GAN (cGAN), may receive an image or images as an input and may obtain or create a depth map for each image or images. In one or more embodiments, an AI network may evaluate obtained one or more images (e.g., a CT image, a bronchoscopic image, etc.), one or more virtual images, and one or more ground truth depth maps to generate depth map(s) for the one or more images and/or evaluate the generated depth map(s). A Three Cycle-Consistent Generative Adversarial Network (3cGAN) may be used to obtain the depth map(s) and/or evaluate the quality of the depth map(s), and an unsupervised learning method (designed and trained in an unsupervised procedure) may be employed on the depth map(s) and the one or more images (e.g., a CT image or images, a bronchoscopic image or images, any other obtained image or images, etc.). Any feature or features of obtaining a depth map or performing a depth map mode of the present disclosure may be used with any of the depth map or depth estimation features as discussed in A. Banach, F. King, F. Masaki, H. Tsukada, and N. Hata, “Visually Navigated Bronchoscopy using three cycle-Consistent generative adversarial network for depth estimation,” Med Image Anal, vol. 73, p. 102164, October 2021, doi: 10.1016/J.MEDIA.2021.102164 (Banach 2021) or the pose estimates or tracking as described in U.S. Prov. Patent Application No. 63/714,676 entitled “Self-Supervised Direct Pose Estimation for Vision-Based Tracking in Navigated Bronchoscopy” filed Oct. 31, 2024 (Kalia 2024), the disclosures of which are incorporated by reference herein in their entireties.

In one or more embodiments, the apparatus/system controller 102 (or any other controller, processor, computer, etc. discussed herein) may operate to perform a computation of one or more lumen (e.g., a lumen computation mode) and/or one or more of the following: a one or more set or predetermined geometric shapes or one or more circles, rectangles, squares, ovals, octagons, and/or triangles fit/blob process, a peak detection, and/or a deepest point analysis. In one or more embodiments, the computation of one or more lumen may include a one or more set or predetermined geometric shapes or one or more circles, rectangles, squares, ovals, octagons, and/or triangles fit/blob process, a peak detection, and/or a deepest point analysis.

For a one or more set or predetermined geometric shapes or one or more circles, rectangles, squares, ovals, octagons, and/or triangles/blob fit technique(s) of the present disclosure, fitting one or more set or predetermined geometric shapes or one or more circles, rectangles, squares, ovals, octagons, and/or triangles or a blob to a binary object may be equivalent to fitting one or more set or predetermined geometric shapes or one or more circles, rectangles, squares, ovals, octagons, and/or triangles or a blob to a set of points. In one or more embodiments, the set of points may be the boundary points of the binary object. Given a set of points (x1, y1), (x2, y2), (x3, y3), . . . , (xn, yn) a circle (x−a)²+(y−b)²=c² may be fit to the points by summing the squares of the distances from the points to the circle:

$$SS ( a , b , c ) = {\sum }_{k = 1}^{n} {( c - \sqrt{{( xi - a )}^{2} + {( yi - b )}^{2}} )}^{2} .$$

However, a circle/blob fit is not limited thereto (as discussed herein, any one or more set or predetermined geometric shapes or one or more circles, rectangles, squares, ovals, octagons, and/or triangles (or other shape(s)) may be used). Indeed, there are several other variations that may be applied as described in D. Umbach and K. N. Jones, “A few methods for fitting circles to data,” in IEEE Transactions on Instrumentation and Measurement, vol. 52, no. 6, pp. 1881-1885, December 2003, doi: 10.1109/TIM.2003.820472, the disclosure of which is incorporated by reference herein in its entirety. For example, circle/Blob fitting may be achieved on the binary objects by calculating their circularity/blob shape as 4πArea/(perimeter)² and then defining the circle/blob radius.

For peak detection and/or deepest point technique(s), one or more embodiments may include same in various ways. For example, peak detection may be performed in a 1-D signal and may be defined as the extreme value of the signal. Similarly, 2-D image peak detection may be defined as the highest value of the 2-D matrix. Herein, a depth map or maps is/are the 2-D matrix in one or more embodiments, and a peak is the highest value of the depth math or maps which may correspond to the deepest point. However, since there might be more than one airway or airways which are represented by different depth value concentrations along the depth map(s) image or images, more than one peak may exist. The depth map or maps produce an image which predicts the depth of the airways; therefore, for each airway, there may be a concentration of non-zero pixels around a deepest point that the neural network, residual network, GANs, or any other AI structure/network discussed herein or known to those skilled in the art predicted. By applying peak detection to all the non-zero concentrations of the 2-D depth map or maps, the peak of each concentration is detected; each peak corresponds to an airway. In one or more embodiments (including the study discussed herein), a GANs (or another AI structure/network) may be used (or was used) to predict the concentration of non-zero pixels.

One or more features discussed herein may be used for performing host and robot communication, error avoidance or removal communication, navigation planning, autonomous navigation, movement detection, and/or control technique(s) for a steerable catheter, continuum robot, imaging device or system, etc. as discussed herein.

Those skilled in the art will recognize that, in general, terms used herein, and especially in the appended claims (e.g., bodies of the appended claims) are generally intended as “open” terms (e.g., the term “including” should be interpreted as “including, but not limited to,” the term “having” should be interpreted as “having at least”, the term “includes” should be interpreted as “includes, but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element, or intervening features or elements may be present. In contrast, when a feature or element is referred to as being “directly connected”, “directly attached”, or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts, and/or sections. It should be understood that these elements, components, regions, parts, and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists”, and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely”, “only”, and the like in connection with the recitation of claim elements, or it may use a “negative” limitation.

Unless specifically stated otherwise, as apparent from the following disclosure, it is understood that, throughout the disclosure, discussions using terms such as “processing”, “computing”, “calculating”, “determining”, “displaying”, or the like, refer to the actions and processes of a processor such as a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computational or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to”, “in response to”, “related to”, “based on”, or other like past-tense adjectives are generally not intended to exclude such variants, unless indicated otherwise.

As known in the field of medical devices, the terms “proximal” and “distal” are used with reference to the manipulation of an end of an instrument extending from the user to an object, target, sample, etc. In this regard, the term “proximal” refers to the portion (e.g., a handle) of the instrument closer to the user, and the term “distal” refers to the portion (tip) of the instrument further away from the user and closer to an object, target, sample, or surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

As used herein the term “bendable medical device” generally refers to a flexible and thin tubular instrument made of medical grade material designed to be inserted through a narrow opening into a bodily lumen (e.g., a vessel, bronchi, etc.) to perform a broad range of medical functions. A catheter may be a bendable medical device. The more specific term “optical catheter” refers to a bendable medical device comprising an elongated bundle of one or more flexible light conducting fibers that may be disposed inside a protective sheath made of medical grade polymer material and having an optical imaging function. A particular example of an optical catheter is a fiber optic catheter which may include a flexible sheath, a coil, and an optical probe or imaging core contained within the coil. In some applications, a catheter may include a “guide catheter” which functions similarly to a sheath. The bendable medical device may be configured for use with one or more tools. For example, a camera may be inserted into the bendable medical device, or a camera or other optical probe may be an integral part of the device. Biopsy instruments may be used with the bendable medical device.

As used herein, the term “endoscope” refers to a rigid or flexible medical instrument which uses light guided by an optical probe to look inside a body cavity, organ, sample, target, etc. A medical procedure, in which an endoscope is inserted through a natural opening, is called an endoscopy. Specialized endoscopes are generally named for how or where the endoscope is intended to be used, such as, but not limited to, the bronchoscope (mouth and lung), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes. Feature(s) and/or embodiment(s) of the present disclosure may be applicable to one or more of the foregoing endoscopes or any other probes or scopes discussed herein or known to those skilled in the art.

The present disclosure generally relates to imaging or medical devices, and it exemplifies embodiments of an optical probe which may be applicable to an imaging apparatus (e.g., an endoscope). The embodiments of the optical probe and portions thereof are described in terms of their state in a three-dimensional space. As used herein, the term “position” may refer to the location of an object or a portion of an object in a three-dimensional space (e.g., three degrees of translational freedom along Cartesian X, Y, Z coordinates); the term “orientation” may refer to the rotational placement of an object or a portion of an object (three degrees of rotational freedom—e.g., roll, pitch, and yaw); the term “posture” may refer to the position of an object or a portion of an object in at least one degree of translational freedom and to the orientation of that object or portion of object in at least one degree of rotational freedom (up to six total degrees of freedom); the term “shape” may refer to a set of posture, positions, and/or orientations measured along the elongated body of the object; and the term “state” may refer, but is not limited, to any orientation, position, posture, or other placement or status information.

### Autonomous Driving

### Perception

In one or more embodiments, autonomous driving method feature(s) of the present disclosure relies/rely on the 2D image from the monocular bronchoscopic camera without tracking hardware or prior CT segmentation in one or more embodiments. A 200×200 pixel grayscale bronchoscopic image serves as input for a deep learning model (3cGAN (see e.g., Banach 2021, or Kalia 2024, the disclosures of which are incorporated by reference herein in their entireties)) that generates a bronchoscopic depth map and/or pose estimate.

Specifically, 3cGAN's adversarial loss accumulates losses across six levels:

$${L}_{{gan}_{6}} = {L}_{{gan}_{lev}} + {L}_{{gan}_{lev}} + ⋯ + {L}_{{gan}_{lev}} \qquad ( 1 )$$

the adversarial loss of level i is referred to as L_{gan}_{ₗₑᵥᵢ}.

The cycle consistency loss combines the cycle consistency losses from all three level pairs:

$${L}_{cyc} 6 = {‖ {A}_{1} - {G}_{{B}_{2} {A}_{2}^{⋀}} ( {\hat{B}}_{1} ) ‖}_{L 2} + {‖ {B}_{2} - {G}_{{A}_{1} {B}_{1}^{⋀}} ‖}_{L 2} + {‖ {C}_{3} - {G}_{{B}_{4} {C}_{4}^{⋀}} ( {\hat{B}}_{3} ) ‖}_{L 2} + {‖ {B}_{4} - {G}_{{C}_{3} {B}_{3}^{⋀}} ( {\hat{C}}_{4} ) ‖}_{L 2} + {‖ {A}_{5} - {G}_{{C}_{6} {A}_{6}^{⋀}} ( {\hat{C}}_{5} ) ‖}_{L 2} + {‖ {C}_{6} - {G}_{{A}_{5} {C}_{5}^{⋀}} ( {\hat{A}}_{6} ) ‖}_{L 2} \qquad ( 2 )$$

$${L}_{cyc} 6 = {‖ {A}_{1} - {G}_{{B}_{2} {A}_{2}^{⋀}} ( {\hat{B}}_{1} ) ‖}_{L 2} + {‖ {B}_{2} - {G}_{{A}_{1} {B}_{1}^{⋀}} ‖}_{L 2} + {‖ {C}_{3} - {G}_{{B}_{4} {C}_{4}^{⋀}} ( {\hat{B}}_{3} ) ‖}_{L 2} + {‖ {B}_{4} - {G}_{{C}_{3} {B}_{3}^{⋀}} ( {\hat{C}}_{4} ) ‖}_{L 2} + {‖ {A}_{5} - {G}_{{C}_{6} {A}_{6}^{⋀}} ( {\hat{C}}_{5} ) ‖}_{L 2} + {‖ {C}_{6} - {G}_{{A}_{5} {C}_{5}^{⋀}} ( {\hat{A}}_{6} ) ‖}_{L 2} \qquad ( 2 )$$

where A stands for the bronchoscopic image, B stands for the depth map, C stands for virtual bronchoscopic image, \hat{X} represents estimation of X, and the lower index i stands for the networks level.

The merging loss of the 3cGAN combines all the networks levels:

$${L}_{m} 6 = {‖ {B}_{2} - {G}_{{A}_{1} {B}_{1}^{⋀}} ( {G}_{{C}_{6} {A}_{6}^{⋀}} ( {G}_{{B}_{4} {C}_{4}^{⋀}} ( {G}_{{C}_{3} {B}_{3}^{⋀}} ( {G}_{{A}_{5} {C}_{5}^{⋀}} ( {G}_{{B}_{2} {A}_{2}^{⋀}} ( {\hat{B}}_{2} ) ) ) ) ‖}_{L 2} \qquad ( 3 )$$

$${L}_{m} 6 = {‖ {B}_{2} - {G}_{{A}_{1} {B}_{1}^{⋀}} ( {G}_{{C}_{6} {A}_{6}^{⋀}} ( {G}_{{B}_{4} {C}_{4}^{⋀}} ( {G}_{{C}_{3} {B}_{3}^{⋀}} ( {G}_{{A}_{5} {C}_{5}^{⋀}} ( {G}_{{B}_{2} {A}_{2}^{⋀}} ( {\hat{B}}_{2} ) ) ) ) ‖}_{L 2} \qquad ( 3 )$$

The total loss function of the 3cGAN is:

$${L}_{6} = {L}_{{gan}_{6}} + {L}_{{cyc}_{6}} + {L}_{{m}_{6}} \qquad ( 4 )$$

The 3cGAN model underwent unsupervised training using bronchoscopic images from phantoms derived from segmented airways. Bronchoscopic operations to acquire the training data were performed using a Scope 4 bronchoscope (Ambu Inc, Columbia, MD), while virtual bronchoscopic images and ground truth depth maps were generated in Unity (Unity Technologies, San Francisco, CA). The training ex-vivo dataset contained 2458 images. The network was trained in PyTorch using an Adam optimizer on 50 epochs with a learning rate of 2·10⁻⁴ and a batch size of one. Training time was approximately 30 hours, and less than 0.02 s for the inference of one depth map on a GTX 1080 Ti GPU.

In the inference process the depth map was generated from the 3cGAN models by inputting the 2D image from the bronchoscopic camera. The bronchoscopic image and/or the depth map was then processed for airway detection using a combination of blob detection, thresholding, and peak detection. Blob detection was performed on a depth map where 20% of the deepest area was thresholded, and the centroids of the resulting shapes were treated as potential points of advancement for the robot to bend and advance towards. Peak detection was performed as a secondary detection method to detect airways that may have been missed by the blob detection. Any peaks detected inside an existing detected blob were disregarded. Direction vector control command may be performed using the directed airways to decide to employ bending and/or insertion, and/or such information may be passed or transmitted to software to control the robot and to perform autonomous advancement.

As shown in FIGS. 1-9B, one or more embodiments of the present disclosure may be a robotic bronchoscope/endoscope/bendable device using a robotic catheter and actuator unit, a robotic arm, and/or a control software or a User Interface. Indeed, one or more robotic bronchoscopes/endoscopes/bendable devices may use any of the subject features individually or in combination.

While the user may select or change the target path at any point in the planning step, as the autonomous system move into the control step, the user can optionally adjust the target path as described herein, or the prior target path can be used until the predetermined insertion depth or end point is reached. The need for additional selection of the target path can depend on the branching of the lumen network, where, for example, the user provides target path information as each branch within the airway becomes visible in the camera image.

While color is used in this example to indicate the selection of the target path, other colors or other indicators may be used as well, such as a bolder indicator, a flashing indicator, removal of the indicator around the non-selected path lumen, etc.

By having the user output device and displaying symbols for the paths and user instruction GUI with endoscope view in the user output device, user can form their intention intuitively and accurately with the visual information in one place. The dedicated user instruction GUI allows the user to select the target path immediately even when the paths are more than two.

By having the user output device and displaying symbols for the paths and differentiating the symbol for the target path from the other paths with endoscope view in the user output device, the user can form intention intuitively and accurately with the visual information in one place. Particularly, the differentiating the symbol for the target path achieve the user instruction with the minimal symbols without the dedicated user instruction GUI and allows the user to learn/understand how to read symbols with the minimal effort.

Circles (or ovals) are used as the symbol of the paths on the 2D interface, and the cursor provides a symbol for input of user instruction to the GUI. These GUIs have minimal obstacles for the endoscope view in the user output device. Also, selecting object with the cursor are very familiar maneuver from common computer operation, the user can easily learn how to use it.

By pausing the actuator and the linear translation stage during user's instruction, the system can reduce the risk where the user may miss the target path in their interaction. Also, this gives users to think and judge the target path among the paths without pressurizing the user to make decisions in the short time.

By allowing the user to add a new target path if the user cannot find the target path among the existing paths and find the target path in the endoscope view, the plan generated by the system becomes more accurate with minimal effort of the user.

### Voice Input and Visualization

In some embodiments, the user input device is a voice input device. Since the autonomous system is driving the steerable catheter, full directional control of the catheter is unnecessary for autonomous driving. It can also be unwanted. A limited library of commands that the user can provide gives full control to the user to select which lumen is the correct one for the next navigation step, but prevents the user from, for example, trying to keep the steerable catheter in the center of the lumen as they would do with a manual catheter since this can be accomplished through the autonomous function. The limited library also simplifies the system.

Effective voice commands can be in the form of a limited library in the system and are, for the one system as provided herewith are (1) start, (2) stop, (3) center, (4) up, (5) down, (6) right, (7) left, and (8) back. Other systems may have more or fewer commands. Some systems will have a different selection of commands, depending on the use for the steerable catheter. In this embodiment, voice commands are classified as one of the effective commands and acted on as such. Verbalizations that are not classified as one of the effective commands are ignored when the sent command is not recognized as the effective commands. In some embodiments, the user or users train the system to recognize their particular enunciation of the effective voice commands. In other embodiments, the system is pre-set with the range of enunciations that are effective commands.

In some embodiments, the instructions are limited to the eight commands listed above or variants thereof. In other embodiments, the instructions are limited to less than or equal to 4, 6, 8, 10, 12, 14, 16, 18, 20, or 24 commands. The commands may all be limited to instructions for selecting a target path in a lumen.

Some embodiments provide autonomous navigation with voice command as discussed in U.S. Prov. Pat. App. No. 63/742,346, filed on Jan. 6, 2025, which is incorporated by reference herein in its entirety.

In some embodiments, in order for the user to select an airway (or a lumen) for the steerable catheter to move in, the user sends one of voice commands from the options of “center”, “up”, “down”, “right” and “left”. When the voice command is accepted by the system, the color of “x” mark on the selected location may be changed from black to red (or from a first color to a second color) and a triangle (or other geometric shape) may be displayed on the selected mark.

The selected location may stay at the same location until a different location is accepted to the system. As the default, the “x” mark on the center is set when the autonomous navigation mode is started.

The system may set the closest airway from the selected x mark as the airway to be aimed based on the distance between the selected x mark and each diamond mark in the detected airways.

By choosing the closest airway as an operator's intended airway from a “+” (or other set or predetermined) mark, which the operator may move with the voice commands, the operator may instruct the intended airway intuitively and accurately. The operator can always clearly confirm the distance between the “+” mark and the intended airway on the display and easily understand which airway option the autonomous system will choose on the display. Therefore, this transparency gives the operator predictable system behavior and operation confidence during autonomous operation.

Also, since the position options of “+” mark include a limited number, the operator can determine the next position option easily and quickly.

Moreover, with this method, the autonomous system always has at least one intended airway until there is at least one airway candidate. This feature avoids the situation without the intended airway and make the system behavior robust.

The user can stop the autonomous navigation any time by sending a voice command, “stop”, and can start the manual navigation using a handheld controller/operating portion/input device 105 to control the steerable catheter and the linear translational stage. When the user takes over the control, “Manual navigation mode” is displayed on the main display 101-1. The user can restart the autonomous navigation when needed by sending a voice command, “start”. Alternatively, the user can stop the autonomous navigation by, for example, an interaction with the handheld controller.

While the robotic platform is bending the steerable catheter and moving the linear translational stage forward, all input signals to the robotic platform are recorded in the data storage memory HDD 150 (or other memory discussed herein) regardless of navigation modes. When the user needs to retract the robotic platform, the user sends a voice command, “back”, then the robotic platform inversely applies the recorded input signals taken during insertion to the steerable catheter and the linear translational stage. During retraction, the system displays “back” on the display.

(Backup and alternative systems) Instead of sending voice commands, the user may send the commands using other input devices including a number pad or a general computer keyboard. The commands may be assigned as numbers on the number pad or other letters on the keyboard. The other input devices may be used along with or instead of voice commands. In some embodiments, the input device has a limited number of keys/buttons that may be pressed by the user. For example, a numerical keypad is used where 2, 4, 6, and 8 are the four directions and 5 is center. The additional numbers on the keypad may be without function, or they may provide an angled movement.

### Navigation/Driving

The integrated control using first-person view, grants physicians the capability to guide the distal section's motion via visual feedback from the robotic bronchoscope. For forward motion/navigation, users may determine only the lateral and vertical movements of the third (e.g., most distal) section, along with the general advancement or retraction of the robotic bronchoscope. The user's control of the third section may be performed using the computer mouse and drag and drop a cross or plus sign (or other set or predetermined sign) to the desired direction. A voice control may also be implemented additionally or alternatively to the mouse-operated cross or sign. For example, an operator or user may select an airway for the robotic bronchoscope to aim using voice recognition algorithm (VoiceBot, Fortress, Ontario, Canada) via a headset (J100 Pro, Jeeco, Shenzhen, China). The options acceptable as input commands to control the robotic bronchoscope were the four cardinal directions (up, down, left, right, and center) and start/stop. For example, when the voice recognition algorithm accepted “up”, a cross 1003 was shown on top of the endoscopic camera view. Then, the system automatically selected the closest airway to the mark out of the airways detected by the trained 3cGAN model, and sent commands to the robotic catheter to bend the catheter toward the airway.

Additionally or alternatively, any feature of the present disclosure may be used with features, including, but not limited to, training feature(s), autonomous navigation feature(s), artificial intelligence feature(s), etc., as discussed in International Patent Application No. PCT/US2024/037935, filed Jul. 12, 2024, the disclosure of which is incorporated by reference herein in its entirety; International Patent Application No. PCT/US2024/037924, the disclosure of which is incorporated by reference herein in its entirety; International Patent Application No. PCT/US2024/037930, the disclosure of which is incorporated by reference herein in its entirety; and U.S. Prov. Pat. App. No. 63/742,346, filed on Jan. 6, 2025, the disclosure of which is incorporated by reference herein in its entirety.

### Control

For specifying the robot's movement direction, the target airway may be identified based on its center proximity to the user-set marker visible as the cross or cross/plus (or other set or predetermined) sign (which may be any set or predetermined color) in one or more embodiments. In one or more embodiments, a direction vector may be computed from the center of the depth map to the center of this target detected airway. In one or more embodiments, the vector may inform a virtual gamepad controller (or other type of controller) and/or one or more processors, instigating or being responsible for the bending of the bronchoscopic tip. In one or more embodiments, a desired target airway may be selected using voice control, a click/touch on an image, an instruction being sent directly to the controller/processor, etc. In one or more embodiments, the robot may advance in a straight line if this direction vector's magnitude is less than 30% of the camera view's width, which is called linear stage engagement (LSE). In one or more embodiments, the process may repeat for each image frame received from the bronchoscopic camera without influence from previous frames. In one or more embodiments, the bronchoscopic robot may maintain a set or predetermined/calculated linear speed (e.g., of 2 mm/s) and a set or predetermined/calculated bending speed (e.g., of 15 deg/s).

One or more features of the present disclosure may provide at least one or more of the following benefits/advantages: (i) having direct communication (e.g., between a host and a robot or robotic catheter, for example) reduces latency and increases responsiveness and accuracy of position/posture/orientation/state/etc. information being used for commands/inputs/control; (ii) having an ability to move the stage backwards and to command bending of other catheter section(s) (e.g., simultaneously or separately); (iii) having more precision over when/where/how certain motion commands are applied; (iv) having improved smoothness of motion and a reduction in navigation time by bending catheter section(s) and moving a stage simultaneously; (v) addressing situations where a catheter may not be able to align with an airway by employing retracting in one or more embodiments; (vi) reducing or minimizing situations where control command(s) is/are sent based on an error, a communication error (e.g., by or between the host controller and/or the robot controller), or inaccurate information; (vii) identifying situations which are pertinent to autonomous control, such as breathing motion and the entering of a new generation; and/or (viii) improving alignment timing and accuracy by sending a larger motion in a case where a target is farther away and by sending a more refined motion when a target is closer.

One or more embodiments may employ a command set and communication method(s) or technique(s). The location of the target within the 2D image may determine the control commands to be relayed, ultimately, to the one or more controllers which drive the movement of the catheter. These control commands may be represented as gamepad commands, serial commands, or any other form of communication/message in which the controllers may convert to catheter movements. These movements may be one or more of stage insertion/retraction, catheter tip bending, catheter tip displacement, or other catheter section bending/displacement/state change. As aforementioned, direct communication reduces latency and increases responsiveness, and an ability to move the stage backwards and to command bending of other catheter section(s) may be employed.

One or more embodiments may use command thresholding. The direction vector's magnitude may be included in the determination of which control commands to send. In some embodiments, there may be a distinct threshold (e.g., 30% of the camera view's width) or threshold range wherein a bending command may be utilized in a case where the magnitude falls upon one side of the threshold or threshold range, and a linear stage insertion command may be utilized in a case where the magnitude falls on the other side of the threshold or threshold range. As aforementioned, one or more embodiments may: introduce more precision over when/where/how certain motion commands are applied, improve smoothness of motion and reduce navigation time by bending and moving a stage simultaneously, and/or address situations where a catheter may not be able to align with an airway, a lumen, a vessel, etc. by using retraction.

In one or more embodiments, there might also be additional thresholds upon which no commands are sent when the magnitude falls upon a particular side. In other embodiments, a linear stage retraction command can be designated when the magnitude is within a particular threshold range. In this case, if the target is far to the edge of the image, retraction might help bring it closer towards the center of the field of view. A reverse IFTL (rFTL) algorithm (which may operate as the FTL algorithm working in reverse or to move a catheter in reverse) activated during retraction may also help with the control as the rFTL technique(s) may operate to re-orient the catheter sections in a manner in which there is less impedance in the bending required to align with the target airway, lumen, vessel, etc.

Additionally, any command may reside within any threshold level. Also, more than one command may be utilized within the same threshold area. The size of the thresholds and the corresponding commands may vary either through user input (e.g., manually), automatically based on one or more conditions (like the control mode, catheter pose(s), insertion depth, direction vectors angle, etc.), or a combination of one or more of the same.

In other embodiments, there might be multiple threshold frames which can be overlapping. For example, Insertion can have a maximum threshold of 35% of the camera views width, while Bending can have a minimum threshold of 25% of the camera views width. In this scenario, both bending and insertion will be commanded when the magnitude falls within 25% and 35% of the camera views width. Under these conditions, the utilize of FTL will assist with the bending motion as the middle and proximal sections will also begin to guide the tip towards the target airway, lumen, vessel, etc.

In one or more embodiments, target history technique(s) may be used. The process may repeat for each image frame received from the bronchoscopic camera with or without influence from previous one or more frames. In one or more embodiments, the difference in location of the target airway, lumen, vessel, etc. may affect the control determination. For example, in a case where there is a very large displacement of the target airway, lumen, vessel, etc. (and/or it is very close to an unselected airway, lumen, vessel, etc. in the earlier frame), it may be determined to be a misdetection, and no movement may be commanded until the next frame. Similarly, in a case where the magnitude of the target airway's/lumen's/vessel's/etc. direction vector increases, it may indicate breathing motion, and the control output may be muted or subdued until the magnitude and/or the breathing motion returns to an acceptable range. In one or more embodiments, a target airway, lumen, vessel, etc. near the center of the camera image may no longer be found in later frames, and it might be assumed that a new branch has been entered. Such a situation may be informed to a user, and the situation and information may affect the control determination. As aforementioned, one or more embodiments may reduce situations where the control commands are sent based on an incorrect airway, lumen, vessel, etc. and may identify situations which are pertinent to autonomous control, such as breathing motion and the entering of a new generation (e.g., of airway(s) or other target pathway(s)). Data (e.g., 3D) data may be combined as a catheter progresses to generate a bigger, more accurate map of an entire pathway in one or more embodiments.

The threshold levels which determine how the direction vectors change affect the controls, as well as which of the corresponding controls represented by these thresholds, may be fixed/predetermined, adjusted manually from user input, adjusted automatically based on prior changes or other one or more conditions, or a combination of such features.

One or more embodiments of the present disclosure may use command speed and magnitude features or techniques. The speed and magnitudes of the insertion, retraction, and bending commands relayed to the controller(s) or one or more processors may be fixed/predetermined, adjusted by user input, calculated/parametric, or any combination thereof. In one or more embodiments, the speed and/or magnitude of the motion commands may depend on the direction vectors magnitude. Similarly, in one or more embodiments, these command levels may be proportional to the position of the target airway within its corresponding threshold or threshold range. In such scenarios, the threshold may be considered to represent a gradient of speed and/or magnitude of motion commands. The gradient may be discrete or continuous, and the slope or range size may follow a mathematical formula (e.g., linear, logarithmic, exponential, any other predetermined formula, any other formula selected by a user, etc.), may follow a parameterized function based on one or more control conditions, may be user defined, or may be any combination thereof. This gradient may also have ranges within where the slope/size calculation differs from other ranges. Again, the determination of the size/position of these ranges, as well as the corresponding slope/size calculations, may be a generic formula, may be parameterized based on one or more robot conditions, may be user defined, or may be any combination of such features. As aforementioned, one or more embodiments may improve alignment timing and accuracy by sending a larger motion (e.g., a command for same) in a case where a target is further away (e.g., over a distance threshold, equal to or over a distance threshold, etc.) and by sending a more refined motion (e.g., a command for same) in a case where the target is closer (e.g., under a distance threshold, under or equal to a distance threshold, etc.).

In one or more embodiments, all possible command which controllers (e.g., a joystick, a gamepad controller, one or more processors, etc.) accept may be utilized. In one or more embodiments, commands may be combined and more control may exist over when/how/where each individual command is activated/deactivated. One or more embodiments may use past states for error detection and control decisions. One or more embodiments may react to a target location within an image or frame (e.g., within a camera frame or image) by sending or commanding a predetermined, set, or appropriate amount of motion.

Simultaneously, in one or more embodiments, the movements of the initial two sections (first and second sections) may be managed by the FTL motion algorithm, based on the movement history of the third section. During retraction, the reverse FTL motion algorithm may control all three sections, leveraging the combined movement history of all sections recorded during the advancement phase, allowing users to retract the robotic bronchoscope whenever necessary. By applying FTL, a most distal segment may be actively controlled with forward kinematic values, while a middle segment and another middle or proximal segment (e.g., one or more following sections) of a steerable catheter or continuum robot move at a first position in the same way as the distal section moved at the first position or a second position near the first position. The FTL algorithm may be used in addition to the robotic control features of the present disclosure. For example, by applying the FTL algorithm, the middle section and the proximal section (e.g., following sections) of a continuum robot may move at a first position (or other state) in the same or similar way as the distal section moved at the first position (or other state) or a second position (or state) near the first position (or state) (e.g., during insertion of the continuum robot/catheter, by using the navigation, movement, and/or control feature(s) of the present disclosure, etc.). Similarly, the middle section and the distal section of the continuum robot may move at a first position or state in the same/similar/approximately similar way as the proximal section moved at the first position or state or a second position or state near the first position (e.g., during removal of the continuum robot/catheter). Additionally or alternatively, the continuum robot/catheter may be removed by automatically and/or manually moving along the same or similar, or approximately same or similar, path that the continuum robot/catheter used to enter a target (e.g., a body of a patient, an object, a specimen (e.g., tissue), etc.) using the IFTL algorithm, including, but not limited to, using FTL with the one or more control, depth map-driven autonomous advancement, or other technique(s) discussed herein. Other FTL features may be used with the one or more features of the present disclosure.

One or more depth maps may be determined using AI features. For example, one or more embodiments may receive/obtain one or more bronchoscopic images (which may be input into a 3cGAN or any other AI-related architecture/structure for processing) such that a network (e.g., a neural network, a 3cGAN, a GAN, a convolutional neural network, any other AI architecture/structure, etc.) and/or one or more processors may estimate a depth map from the one or more bronchoscopic images. An airway/pathway detection algorithm or process may identify the one or more airways/pathways in the bronchoscopic image(s) and/or in the depth map (e.g., such as, but not limited to, using thresholding, blob detection, peak detection, and/or any other process for identifying one or more airways/pathways as discussed herein and/or as may be set by a user and/or one or more processors, etc.). A direction vector control command may decide between bending and insertion. The direction vector may then be sent to the robot's control software by a virtual gamepad (or other controller or processor) which may initiate the autonomous advancement. At least one embodiment may have a network estimate a depth map from a bronchoscopic image, and the airway/pathway detection algorithm(s) may identify the airways/pathways.

In some embodiments, when the user starts the autonomous navigation, an indicator of the navigation mode being used, such as displaying “Autonomous navigation mode” on the main display 101-1. The autonomous navigation system may detect lumen or airways/pathways in the camera view.

In some embodiments, the user has the ability to stop the autonomous navigation anytime by pushing a button on the handheld controller/operating portion/input device 105 and can start the manual navigation to control the steerable catheter and the linear translational stage by the handheld controller. When the user takes over the control, an indicator of this control is provided, such as a display of “Manual navigation mode” on the main display 101-1. The user can restart the autonomous navigation when needed by, for example, pushing a button on the handheld controller/operating portion/input device 105.

When the steerable catheter reaches a position close to the region of interest (e.g., tumorous tissue, target, sample, object, etc.), the user may have the option to switch the navigation mode to the manual navigation and, for example, deploy a biopsy tool toward the region of interest to take a sample through the working tool access port.

A Return to the Carina (or other set or predetermined location) function may be included with the autonomous driving catheter and system. While the robotic platform is bending the steerable catheter and moving the linear translational stage forward, all input signals to the robotic platform may be recorded in the data storage memory HDD 150 (see also, hard disk 1204, SSD 1207, or any other data storage known to those skilled in the art, etc. that may be used to record data) regardless of navigation modes. When the user indicates a start of the Return to Carina (or other set or predetermined location) function (e.g., hitting the appropriate button on the handheld controller/operating portion/input device 105), the robotic platform inversely applies the recorded input signal taken during insertion to the steerable catheter and the linear translational stage.

In some embodiments, an insertion depth may be set before driving the steerable catheter. When the linear translational stage reaches the predetermined insertion depth or end point, the autonomous navigation system can be instructed to stop bending the steerable catheter and moving the linear translational stage forward. The robotic platform then switches the mode from the autonomous navigation to the manual navigation. This allows the user to start interacting with the region of interest (e.g., take a biopsy, take an image, perform another procedure, etc.) or to provide additional adjustments to the location or orientation of the steerable catheter.

In some embodiments, the frame rate is set for safe movement. Then based on the frame rate and the acceptable risk during bronchoscopy (or other predetermined procedure or imaging), the speed of bending the steerable catheter and the speed of moving the linear translational stage are decided. If it is important to move the steerable catheter based on the images when the steerable catheter is moving faster than can be ‘seen’ by the images from the camera. In an example where the maximum frame rate is 10 frames per second (fps) and the acceptable amount of the airway pushed by the steerable catheter is 0.5 mm, the speed of the linear translational stage may be set less than 5 [mm/sec]. Other frame rates and risk factors will suggest different speeds.

One or more embodiments of the presented method of the present disclosure may be dependent on the quality of bronchoscopic depth estimation by 3cGAN (see e.g., Banach 2021), pose estimation by vision based tracking (see e.g., Kalia 2024), or other AI-related network architecture used or that may be used (for example, while not limited hereto: in one or more embodiments, in a case where one or more processors train one or more models or AI-networks, the one or more trained models or AI-networks is or uses one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel or lumen and/or including tissue characterization data during pullback in a vessel or lumen, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, a model using repeated object detection or regression model technique(s); one or more other AI-networks or models known to those skilled in the art; etc.).

FIG. 7A is an apparatus/system block diagram of at least one embodiment example of a bendable medical and/or imaging device apparatus/system 1000 discussed herein. In one or more embodiments, and while not limited hereto, the apparatus/system 1000 may be set up as shown in one or more of FIGS. 1-9B. For example, the bendable medical device apparatus/system 1000 may comprise: the actuator or driving unit 103 (also referred to herein as a “driver”) for driving the wires 160; the base platform 108, the linear translation stage 122, and/or the rail 110; the bendable imaging or medical device 104 (also referred to herein as a “catheter 104” or a “continuum robot 104”), a positioning cart (e.g., having the display controller 100, the controller 102, etc.), and an operation console or computer 1200 (also referred to herein as a “controller 1200” or a “processor 1200”), which may further include or comprise, in one or more embodiments, push-button, thumbstick, and/or joystick operational console or controller features and navigation software (e.g., software 16 on a display 101, 101-1, 101-2, 1209, any other display discussed herein, etc.). The bendable medical and/or imaging device apparatus/system 1000 may operate to interact with external system component(s) and clinical user(s) to facilitate use in a patient or patients.

FIG. 7B illustrates a general structure that may be used in one or more embodiments for the steerable medical apparatus/system 1000 from FIG. 1 in a functional block diagram without the user U and/or the patient P. The medical apparatus/system 1000 may include a handle/operating portion/input device 105 and a bendable medical device 104, which are removably connected to each other by a connector assembly 50. The handle/operating portion/input device 105 includes an actuator system 103 that is part of the driving unit 310 and which operates to receive electronic commands from the computer system 100, 102, 1200, 1200′ (or any other processor or computer discussed herein) to mechanically actuate the bendable medical device 104. The driving unit 310 may include motors 1-M (or motors 144). Each motor may be attached to a connector 210 that connects each motor to a respective drive wire 160, or each motor may be directly attached to a respective drive wire 160 in one or more embodiments. The actuator 103 may include a controller or processor 320 that operates to control the motors 1-M, 144 and/or the send signals to/from the connectors 210 and/or the drive wires 160 (e.g., via a line 305 and/or a splitter or electronic component 304 that operates to connect the controller 320 to each of the connectors 210 and/or the drive wires 160). The handle/operating portion/input device 105 operates to be detachably mounted on the robotic platform 108 and/or the stage 122 (and/or the rail 1100), which may be part of the positioning cart. The handle/operating portion/input device 105 may include a dial or knob 252 for sending signals and/or commands via the handle/operating portion/input device 105. The robotic platform 108 may include a robotic arm 132, the rail 110, and/or the stage 122 for robotically guiding the bendable medical device 104 towards a target site 82 within the subject or patient P. When the handle/operating portion/input device 105 is not mounted on the robotic platform 108, the handle/operating portion/input device 105 may be operated manually by the user U to control the bendable medical device 104. For treating or examining the patient P, the steerable medical apparatus/system 1000 may include one or more access ports 126 arranged on or around the handle/operating portion/input device 105. Access ports 126 may be used for inserting end effectors or for passing fluids to/from the patient P. In one or more embodiments, the bendable device 104 may include a camera 106 and/or one or more electromagnetic (EM) field sensors to provide a field of view FOV. An EM generator 107 may interact with one or more EM sensors 106 arranged on the bendable medical device 104 for tracking the position, shape, and/or orientation of the bendable medical device 104 while being inserted through a bodily lumen 81 towards the target site 82 within the patient P. The bendable device 104 may include the proximal section 148 and/or one or more bending sections 152, 254, and/or 156 (and/or one or more sub-sections 1, 2, 3, . . . N or more) that may be arranged along a longitudinal axis (Ax). The proximal section 148 may be a non-steerable section and may serve to connect the steerable/bendable section(s) of the bending device 104 to the handle/operating portion/input device 105 and the actuator 103.

In one or more embodiments, the steerable distal section 156 (and the middle section 154) may be divided into multiple bending segments 1, 2, 3 . . . N, which operate to be bent, curved, twisted, and/or rotated when advancing the bendable medical device 104 through intraluminal tortuous paths of a bodily lumen (e.g., such as the lumen 81). Each bending segment includes at least one ring-shaped component. The steerable medical apparatus/system 1000 may operate in a three-dimensional (3D) space defined by a 3D coordinate system of x, y, z Cartesian coordinates. The bendable medical device 104 may define at least one tool channel 168 which extends from the proximal end to the distal end along the longitudinal axis Ax. The bendable medical device 104 may include one or more position and/or orientation cameras and/or sensors 106 arranged on the wall the catheter sheath, and may include a removable imaging device 180, such as a fiber camera or a miniature electronic CMOS sensor arranged in the tool channel 168. In one or more embodiments, the imaging device 180 may be arranged such that its imaging plane is in the x-y plane, and the longitudinal axis Ax of the bendable medical device 104 extends along the z-axis of the coordinate system.

An example of a bendable medical device 104 and a method of using the medical device via the medical apparatus/system 1000 is described in United States Pat. Pub. No. 2019/0105468, which is incorporated by reference herein in its entirety. Other examples of bendable medical devices and methods of using the medical device via the medical system are disclosed in United States Pat. Pub. Nos. 2018/0243900; 2018/0311006; 2019/0105468; 2019/0015978; and 2019/0105468; and PCT Pub. Nos. WO2018/204202; WO/2020/086749; and WO/2020/092096, all of which are incorporated by reference herein in their entireties.

For inserting an endoscope into a biological lumen 81 such as an airway of a patient P, the tip (distal end) of the bendable medical device 104 may be advanced (navigated) along a center line of the lumen 81. In this case, an imaging device 180 (e.g., a miniature camera) and/or a camera 106 may be arranged in the tool channel 168 to provide a live-view image of the lumen 81 taken directly from the instrument's field of view (FOV). However, in some embodiments, the bendable medical device 104 may not allow for the arrangement of a camera within the tool channel 168. In this case, navigation may be provided by intra-procedural guided imaging based on position and/or orientation provided by the one or more sensors 106 arranged along the sheath and/or the device 104. In any case, in order to reach a desired target site 82, the bendable medical device 104 may operate to bend, twist and/or rotate in different directions such that the distal section 156 of the bendable medical device 104 continuously changes shape and direction until it reaches an optimal location aligned with target site 82, such as, but not limited to, a tumor.

In one or more embodiments, the bending, twisting, and/or rotation (steering) of the bendable medical device 104 may be controlled by a system comprised of the handle/operating portion/input device 105, the actuator system 103 and/or the computer system 100, 1200, 1200′ (and/or any other computer or processor discussed herein). The actuator system 103 may include a micro-controller 320 and an actuator unit 310 which may be operatively connected to the computer system 100, 1200, 1200′ via a network connection 425 (e.g., a cable bundle or wireless link). The computer system 100, 1200, 1200′ (and/or any other computer or processor discussed herein) may include suitable software, firmware, and/or peripheral hardware operated by the processor or CPU 102 (and/or any other processor or CPU discussed herein). The computer system 400, the actuator system 300, and the handle 200 may be operatively connected to each other by the robot platform 108, which may include one or more robotic arms 132 and translation stage 122 (and/or the rail 110). In some embodiments, the actuator system 103 may include or be connected to a handheld controller/operating portion/input device 105, 1600, such as, but not limited to, a gamepad controller or a portable computing device like a smart phone or a tablet. Among other functions, the computer system 100, 1200, 1200′ and the actuator system 103 may provide a surgeon or other operator with a graphical user interface (GUI) and patient information shown in the display screen 101-1 to operate the steerable medical system 1000 according to its application(s).

FIG. 8A illustrates the bendable medical device 104 having three or four sections (152, 154, 156, and 148; one or more embodiments may have sections 156, 154, and 148), where the tip 320 of the bendable medical device 104 is the distal-most portion of the distal section 156. The bendable medical device 104 is situation in a body lumen, which can be the lung 190. As indicated by the lung 190, at each bifurcation, the airway may become smaller such that the bendable medical device 104 may no longer fit into the airway.

FIG. 8B illustrates at least one embodiment of an apparatus/system 1000 that operates to control a bendable robot/device 104 based on an instruction received from an input device/operating portion 105. The robot controller (which may be a processor or CPU, such as, but not limited to, processor 100, 102, 1200, 1200′, 1201, any other processor or computer discussed herein, etc.) operates to send information in the form of instructions from the input device/operating portion 105 to a host controller 801 of a host apparatus 800. The host controller 801 may be a processor or CPU, such as, but not limited to, processor 100, 102, 1200, 1200′, 1201, any other processor or computer discussed herein, etc. The host controller 801 may operate to generate a Graphical User Interface (GUI) based on the information of the instructions from the robot controller (e.g., processor 100, 102, 1200, 1200′, 1201, any other processor or computer discussed herein, etc.). The GUI may operate to provide a posture, orientation, state, etc. information of the bendable robot/device 104 to a user U via the display 101-1, 1209, etc. The user U may input the instructions based on the posture, orientation, state, etc. information on the GUI.

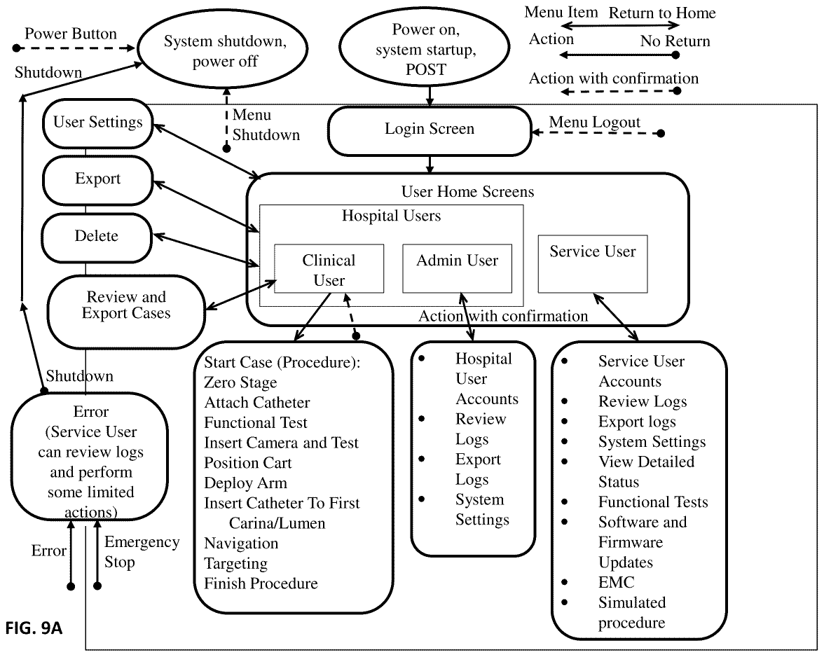

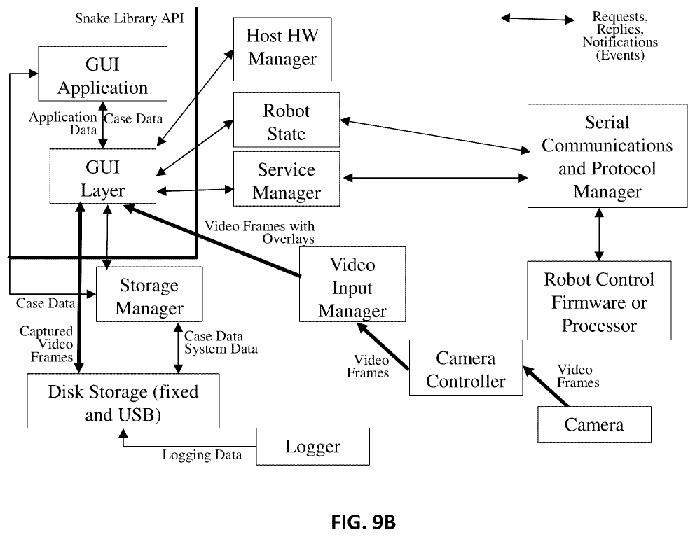

FIG. 9A diagrammatically illustrates a summary of system behaviors for one or more embodiments. By way of a few examples, an apparatus, system 1000 may be turned on or shut down via the Power Button. Once the Login Screen appears on a display 101-1, 1209, etc., a user may login. In one or more situations, there may be a few different user profiles, such as, but not limited to, an admin user, a clinical user, a service user, etc. Depending on the type of user logging in, different functions or features may be provided by the apparatus/system 1000. For example, a clinical user of a hospital may interact with a catheter or other bendable device 104 and perform one or more tests (e.g., a Functional Test, an Insert Camera and Test, etc.), may position a cart, may deploy an arm (e.g., the arm 132), may insert the catheter or bendable device 104 to a first carina or lumen, may perform navigation, may perform targeting, etc. As another example, an admin user of a hospital may review accounts, review logs, export logs, evaluate/update system settings, etc. By way of a further example, a service user may: manage service user accounts, review logs, export logs, review/update system settings, view a detailed status, perform functional tests, manage software and/or firmware updates, may simulate a procedure, and/or may evaluate Electromagnetic Compatibility (EMC), review error logs and perform actions in response, etc.

FIG. 9B diagrammatically illustrates an example of data flow for high level system behaviors for one or more embodiments. One or more embodiments may include a Robot State, a Serial Communications and Protocol Manager (also referred to herein as a “host controller” or “host processor”), and a Robot Control Firmware or processor (also referred to herein as a “robot controller” or “robot processor”). The robot controller or processor (which may be any processor or CPU, such as, but not limited to, 102, 1200, 1200′, 1201, any other processor or CPU discussed herein, etc.) operates to check a communication status with the host controller or processor 801. In a case where the robot controller or processor (e.g., processor or computer 100, 102, 1200, 2100′, 1201, any other processor or computer discussed herein, etc.) detects an error in communication with the host controller or processor 801, the robot controller operates to stop the bendable robot/device 104 from moving. A camera, a camera controller/processor, a video input manager, etc. may be used to send video frames or video frames with overlays to a GUI or GUI layer. The GUI or GUI layer may be used by a host HW manager and/or service manager, and may be used to interact with or update the Robot State. The GUI or GUI layer may be used by a storage manager to monitor case data and/or system data in a storage, and a logger may manage or monitor a logging of data.

In one or more embodiments, a robot controlling apparatus and/or the actuator 103 may include: a first interface 802 that operates to receive an instruction from an input device, the instruction for bending/moving a bendable robot or device; a processor or controller that operates to: (i) control one or more actuators to bend and/or move the bendable robot or device in accordance with the instruction from the input device, and (ii) detect an error in communication between the robot controlling apparatus and a host apparatus and, in a case where the error is detected, restrict the one or more actuators from bending and/or moving the bendable robot or device; and a second interface 803 that operates to transfer information of the instruction from the processor or controller to a host apparatus, wherein a graphical user interface on a display operates to display information of a current posture, position, orientation, and/or state of the bendable robot or device. In one or more embodiments, the robot controlling apparatus further includes or is in communication with the bendable robot or device, such as, but not limited to, a catheter or continuum robot. In one or more embodiments, the robot controlling apparatus further includes the one or more actuators and/or one or more motors that operate to control, move, and/or bend one or more drive wires of the bendable robot or device. In one or more embodiments, the robot controlling apparatus may be in communication with the host apparatus and may transfer the information of the instruction from the processor or controller of the robot controlling apparatus to the host apparatus, and/or the GUI may operate to provide the information of the current posture, position, orientation, and/or state of the bendable robot or device to a user. The user may be a user of the robot controlling apparatus and/or the host apparatus. In one or more embodiments, the error in communication detection may be performed at or using an operating system driver, firmware, and/or software levels of the robot controlling apparatus and/or of the host apparatus. The processor or controller may operate to interact with the operating system to check for any deviancy from a standard communication protocol timing and message length. In one or more embodiments, the processor or controller may operate to interact with the firmware to check that the message contents are within the application command set, and/or the processor may operate to interact with the software to check that the one or more messages (or all messages) sent receive replies within a timeout period.

In one or more embodiments, the processor or controller of the robot controlling apparatus further operates to send, to a processor or controller of the host apparatus, one or more of the following: (i) status information (e.g., whether the catheter is connected, whether the catheter is disconnected, etc.); (ii) navigation information of the bendable robot or device (e.g., information as the user manipulates a tip of a catheter of the bendable robot or device; information as the user manipulates the linear stage (and/or the rail 110, the robotic platform 108, the arm 132, etc.); (iii) one or more requests from the user to change a state (e.g., navigation, targeting, orientation, position, rotation, posture, etc.); (iv) one or more requests from the user to take a snapshot; (v) one or more requests from the user to rotate the camera image; (vi) warning/error/failure information (such as, but not limited to, a voltage being out of a set or predetermined range, a temperature being out of a set or predetermined range, an emergency stop is issued or not, etc.); (vii) version information and parameter values; and/or (viii) whether the information being transmitted is a response to a request from the processor or controller of the host apparatus or is an unsolicited notification.

In one or more embodiments, the processor or controller of the host apparatus further operates to send, to the processor or controller of the robot controlling apparatus, one or more of the following: (i) one or more requests for status, version, voltage, temperature, parameters, etc. (In one or more embodiments, requests for voltage and temperature may also be used as a heartbeat signal sent periodically to check that the bendable robot or device is communicating; (ii) one or more requests to home the linear stage 122 (and/or one or more components connected thereto, such as, but not limited to, the rail 110, the arm 132, the platform 108, etc.); (iii) one or more requests to zero the actuator 103 and/or the actuation units (catheter bending motors 1-M and/or 144); (iv) one or more navigation requests (e.g., from a service user) to manipulate the bending/moving/posture/orientation/state/etc. and/or the linear stage 122 (and/or one or more components attached thereto, such as, but not limited to, the rail 110, the arm 132, the platform 108, etc.) of the catheter/bendable robot or device); (v) a request for a joystick or handle mode (e.g., for normal use, for testing, for procedure, for imaging, for control, for control by a user, etc.); (vi) one or more reports to report error(s) to the processor or controller of the robot controlling apparatus; (vii) one or more requests for shutdown; and/or (viii) information regarding EMC testing parameters and operations.

In one or more embodiments, the processor or controller of the host apparatus may operate to perform or instruct one or more of the following: (i) display of a GUI; (ii) pre-procedure operations (e.g., login, enter case information, etc.); (iii) procedure setup operations (e.g., guide the user through the setup); (iv) procedure operations (e.g., display video from one or more cameras, show catheter/bendable robot or device status (which may include wire forces), issue warnings/warn a user as needed, show additional video feeds, etc.); and/or (v) post-procedure operations (e.g., review cases, export cases to USB memory devices, export cases to one or more memory devices, perform management task(s) (e.g., add/remove users, handle per-user settings, etc.), and/or writing a log to disk/memory).

In one or more embodiments, the processor or controller of the robot controlling apparatus may further operate to perform or instruct one or more of the following: (i) stopping acceptance of the instructions from the input device in a case where the processor or controller of the robot controlling apparatus detects the communication error with the processor or controller of the host apparatus; (ii) stopping transfer, from the processor or controller of the robot controlling apparatus to the processor or controller of the host apparatus, of the information of the instruction input by the input device in a case where the processor or controller of the robot controlling apparatus detects the communication error with the processor or controller of the host apparatus; and/or (iii) controlling an operation mode of the of the bendable robot or device to be a relax mode in a case where the processor or controller of the robot controlling apparatus detects the communication error with the processor or controller of the host apparatus.

In one or more embodiments, the catheter and/or the bendable robot or device may operate in a relax mode to bend in accordance with an external force.

In one or more embodiments, the apparatus/system 1000 may further include an indicator that operates to indicate that an error is happening or has happened, in response to a detection of the communication error with the host controller 801 of the host apparatus 800. In one or more embodiments, the indicator may be disposed in the input device/operating portion 105, in the robot controlling apparatus or the actuator 103, and/or in a display (e.g., the display 101-1, 1209, etc.) that is in communication with or is part of the apparatus/system 1000.

In one or more embodiments, the robot controlling apparatus or the actuator 103 of the apparatus/system 1000 may operate to move along a slider and/or a rail (e.g., the rail 110), in accordance with a control by the host controller 801 of the host apparatus 800. In one or more embodiments, the instruction(s) may include an instruction to move the robot controlling apparatus or the actuator 103 along the slider and/or the rail 110, and the processor or controller of the robot controlling apparatus 103 operates to stop transferring, from the processor or controller of the robot controlling apparatus 103 to the host controller 801 of the host apparatus 800, the information of the instruction corresponding to a movement of the robot controlling apparatus and/or the actuator 103 along the slider or the rail 110, in a case where the processor or controller of the robot controlling apparatus detects the communication error with the host controller 801 of the host apparatus 800.

In one or more embodiments, the processor or controller (e.g., the processor 100, 102, 1200, 1200′, any other processor discussed herein, etc.) of the robot controlling apparatus and/or the actuator 103 may operate to disable the one or more actuator drivers or motors (e.g., the motor(s) 1-M, 144, etc.) to bend or from bending the catheter and/or the bendable robot or device 104 in a case where the processor or controller of the robot controlling apparatus and/or the actuator 103 does not receive a predetermined response and/or a valid message from the host controller 801 of the host apparatus 800 within a predetermined period or amount of time. In one or more embodiments, the predetermined period or amount of time may be one or more of the following: less than 1 second, 1 second, 2 seconds, 3 seconds, 1 minute, 10 minutes, in a range of 1 second to 1 minute, in a range of 1 second to 10 minutes, an amount of time set by a user of the robot controlling apparatus and/or the actuator, an amount of time set by a user of the bendable robot or device or catheter, an amount of time set by a user of the host apparatus, in a range of time set by a user of the robot controlling apparatus or actuator, in a range of time set by a user of the bendable robot or device or the catheter, in a range of time set by a user of the host apparatus, etc. In one or more embodiments, the processor or controller (e.g., the processor 100, 102, 1200, 1200′, any other processor discussed herein, etc.) of the robot controlling apparatus and/or the actuator 103 may operate to disable the one or more motors 1-M, 144, etc. to bend or from bending the catheter and/or the bendable robot or device 104 in a case where the processor or controller (e.g., the processor 100, 102, 1200, 1200′, any other processor discussed herein, etc.) of the robot controlling apparatus and/or the actuator 103 does not receive any valid message from the host controller 801 within a predetermined period or amount of time. In one or more embodiments, the apparatus/system 1000 may further include a camera 180, 106, etc. which is inserted in a lumen of the catheter and/or the bendable robot or device 104. An image or images captured by the camera 180, 106, etc. may be displayed on a display (e.g., the display 101-1, 1209, any other display discussed herein, etc.). In one or more embodiments, the host controller 801 may keep the image captured by the camera 180, 106, etc. being displayed, even in a case where the processor or controller (e.g., the processor 100, 102, 1200, 1200′, any other processor discussed herein, etc.) of the robot controlling apparatus and/or the actuator 103 detects the communication error with the host controller 801. In one or more embodiments, the host controller 801 may be the processor or computer 100, 102, 1200, 1200′, any other processor discussed herein, etc. In one or more embodiments, the predetermined response and/or the valid message may include one or more of the following: a query version string; a request to reset the bendable robot or catheter; a request to run power-on test(s); a request or query for a cyclic redundancy check (CRC) and/or for an on or off status of the CRC; a parameter(s) query; a request to query, notify, or set a failure string; a request to query, notify, or set a warning string; a request to notify or log a string or log string to an application log; a request to shut down the bendable robot or catheter and/or the robot controlling apparatus or actuator; a request to start a new procedure; a request to power cycle; a request for a query of complete status; a request to query, notify or set a mode, a disconnect mode, a disabled mode, a halt mod, a tip control mode, a manual mode, a pause mode, a relax mode, a park mode, a Follow-the-Leader (FTL) mode, a reverse FTL mode, a targeting mode, and/or an error mode; a request to query or notify an emergency stop or E-stop, or to obtain an ok or tripped status of the emergency stop; a request to establish a home, zero, or default position for the bendable robot or catheter for one or more or all motors or drive wires of the robot controlling apparatus or actuator; a request to query or notify a connected or a disconnected status of the bendable robot or catheter; and/or a request for a wire test of the bendable robot or catheter.

The processor or controller (e.g., the processor 100, 102, 1200, 1200′, any other processor discussed herein, etc.) of the robot controlling apparatus and/or the actuator 103 stops accepting the instructions from the input device/operating portion 105 in a case where the processor or controller (e.g., the processor 100, 102, 1200, 1200′, any other processor discussed herein, etc.) of the robot controlling apparatus and/or the actuator 103 detects the communication error with the host controller. As such, one or more embodiments may achieve the advantage or benefit where the processor or controller (e.g., the processor 100, 102, 1200, 1200′, any other processor discussed herein, etc.) of the robot controlling apparatus and/or the actuator 103 operates to put the apparatus/system 1000 and/or the catheter or bendable robot or device 104 in a safety shutdown mode since the user U will not be able to see the results of the user's actions.

In one or more embodiments, the processor or controller (e.g., the processor 100, 102, 1200, 1200′, any other processor discussed herein, etc.) of the robot controlling apparatus and/or the actuator 103 operates to stop transferring, from the processor or controller (e.g., the processor 100, 102, 1200, 1200′, any other processor discussed herein, etc.) of the robot controlling apparatus and/or the actuator 103 to the host controller 801, the information of the instruction input by the input device/operating portion 105, in a case where the processor or controller (e.g., the processor 100, 102, 1200, 1200′, any other processor discussed herein, etc.) of the robot controlling apparatus and/or the actuator 103 detects the communication error with the host controller 801. As such, one or more embodiments may achieve the advantage or benefit avoiding misleading the user U into believing that the apparatus/system 1000 and/or the catheter or robot 104 is in a normal operation (e.g., in case where the communications link is compromised, in a case where the link is in error, in a case where there is a communication error, etc.).

In one or more embodiments, the processor or controller (e.g., the processor 100, 102, 1200, 1200′, any other processor discussed herein, etc.) of the robot controlling apparatus and/or the actuator 103 operates to control an operation mode of the catheter and/or the bendable robot or device 104 to change to or enter into a relax mode in a case where the processor or controller (e.g., the processor 100, 102, 1200, 1200′, any other processor discussed herein, etc.) of the robot controlling apparatus and/or the actuator 103 detects the communication error with the host controller 801. As such, one or more embodiments may achieve the advantage or benefit of having the relax mode operate as a safety mode where the catheter and/or the bendable robot or device 104 may be removed from the patient P regardless of the posture/position/orientation/state/etc. of the robot 104 or the host apparatus 800.

In one or more embodiments, the catheter and/or the bendable robot or device 104 operates to bend in accordance with an external force when in the relax mode. As such, one or more embodiments may achieve or provide the advantage or benefit where the catheter or bendable robot/device 104 operates to conform to the lumen 81 (e.g., an airway, vessel, etc.) of the patient P in a case where the bendable robot/device 104 is removed manually (and may do so while reducing stress on the lumen 81 in a case of an emergency removal).

In one or more embodiments of the apparatus/system 1000 further including the indicator that indicates that the communication error is happening or has happened, in response to the detection of the communication error with the host controller 801, an advantage or benefit may be achieved or provided where the catheter and/or the bendable robot/device 104 operates to independently signal to the user U that the error is occurring or has occurred. This would operate to act as a primary or secondary signal of an error condition to the user U.

In one or more embodiments, the indicator may be disposed in the input device/operating portion 105, in the robot controlling apparatus or the actuator 103, and/or in a display (e.g., the display 101-1, 1209, etc.) that is in communication with or is part of the apparatus/system 1000. One or more embodiments may achieve or provide the benefit of having an additional safety measure where the input device/operating portion 105 also signals to the user U that the apparatus/system 1000 (or any other apparatus/system discussed herein) is in error.

As aforementioned, the robot controlling apparatus and/or actuator 103 may operate to move along a slider or the rail 110 in accordance with a control by the host controller 801. As such, one or more embodiments may provide the advantage or benefit of having the slider or the rail 110 provide precise depth control into the lumen 81 (e.g., an airway, a vessel, another lumen, etc.) for navigation. One or more processors of the apparatus/system 1000 may operate to remember the path the catheter and/or the bendable robot/device 104 has taken and to reverse the path exactly (e.g., using the FTL algorithm, the rFTL algorithm, and/or another navigation algorithm discussed herein).

In one or more embodiments, the instruction(s) may include an instruction to move the robot controlling apparatus or the actuator 103 along the slider and/or the rail 110, and the processor or controller of the robot controlling apparatus 103 operates to stop transferring, from the processor or controller of the robot controlling apparatus 103 to the host controller 801 of the host apparatus 800, the information of the instruction corresponding to a movement of the robot controlling apparatus and/or the actuator 103 along the slider or the rail 110, in a case where the processor or controller of the robot controlling apparatus detects the communication error with the host controller 801 of the host apparatus 800. As such, one or more embodiments may provide the advantage or benefit of stopping or preventing the user U from driving further into the lumen 81 with no visibility into the user's actions in a case where the communication error is happening or has happened.

In one or more embodiments, the processor or controller (e.g., the processor 100, 102, 1200, 1200′, any other processor discussed herein, etc.) of the robot controlling apparatus and/or the actuator 103 may operate to disable the one or more actuator drivers or motors (e.g., the motor(s) 1-M, 144, etc.) to bend or from bending the catheter and/or the bendable robot or device 104 in a case where the processor or controller of the robot controlling apparatus and/or the actuator 103 does not receive a predetermined response from the host controller 801 of the host apparatus 800 within a predetermined period or amount of time. As such, one or more embodiments may provide or achieve the advantage or benefit of stopping or preventing the user U from driving further into the lumen 81 with no visibility into the user's actions.

In one or more embodiments, the processor or controller (e.g., the processor 100, 102, 1200, 1200′, any other processor discussed herein, etc.) of the robot controlling apparatus and/or the actuator 103 may operate to disable the one or more motors 1-M, 144, etc. to bend or from bending the catheter and/or the bendable robot or device 104 in a case where the processor or controller (e.g., the processor 100, 102, 1200, 1200′, any other processor discussed herein, etc.) of the robot controlling apparatus and/or the actuator 103 does not receive any valid message from the host controller 801 within a predetermined period or amount of time. As such, one or more embodiments may provide the advantage or benefit of stopping or preventing the user U from bending, moving, or changing a state of the catheter and/or the bendable robot/device 104 while having no visibility into the user's actions.

In one or more embodiments, the apparatus/system 1000 may further include a camera 180, 106, etc. which is inserted in a lumen of the catheter and/or the bendable robot or device 104. As such, one or more embodiments may provide the advantage or benefit of having a camera inserted into the catheter and/or the bendable robot/device 104 to provide a live video feed for navigation (e.g., of the lumen 81, of an airway of the patient P, of a vessel, etc.), and images may also be captured for reference.

An image or images captured by the camera 180, 106, etc. may be displayed on a display (e.g., the display 101-1, 1209, any other display discussed herein, etc.). As such, one or more embodiments may achieve or provide the benefit or advantage of display the image on a display to provide the live video feed for navigation (e.g., of the lumen 81, of an airway of the patient P, of a vessel, etc.).

In one or more embodiments, the host controller 801 may keep the image captured by the camera 180, 106, etc. being displayed, even in a case where the processor or controller (e.g., the processor 100, 102, 1200, 1200′, any other processor discussed herein, etc.) of the robot controlling apparatus and/or the actuator 103 detects the communication error with the host controller 801. As such, one or more embodiments may achieve or provide the advantage or benefit of having a camera (e.g., the camera 180, another camera discussed herein, etc.) be connected to the host apparatus 800 (and may not be connected to the catheter and/or the robot/device 104), the live video feed may continue to display even in a case where an error occurs. This allows the user U to view the navigation (e.g., of the lumen 81, of an airway of the patient P, of a vessel, etc.) as the user U operates, moves, removes, etc. the catheter and/or the robot/device 104.

In view of the above, one or more embodiments of the apparatus/system 1000 (and/or the robot controlling apparatus and/or the actuator 103) may operate to check a communication status with the host controller 801 of the host apparatus 800. By way of a few examples, the processor or controller (e.g., the processor 100, 102, 1200, 1200′, any other processor discussed herein, etc.) of the robot controlling apparatus and/or the actuator 103 may operate to do one or more of the following: (i) stop accepting instructions from the input device/operating portion 105 when an error occurs (e.g., with the host controller); (ii) stop transferring (e.g., from the actuator robot controller of the actuator 103 to the host controller 801) the information of the instruction input by the input device/operating portion 105 when an error occurs (e.g., with the host controller 801); and/or (iii) control/switch/change an operation mode of the catheter and/or bendable robot/device 104 to be a relax mode when an error occurs (e.g., with the host controller). By way of a couple of additional examples, the bendable robot 104 may bend in accordance with an external force in the relax mode, and/or the apparatus/system 1000 may further include the indicator to indicate when an error is happening or has happened in response to a detection of an error (e.g., a communication error, an error with the host controller, etc.). In one or more embodiments, the input device 1600 may be used.

In view of the above, one or more embodiments of the apparatus/system 1000 (and/or the robot controlling apparatus and/or the actuator 103) and/or the processor or controller (e.g., the processor 100, 102, 1200, 1200′, any other processor discussed herein, etc.) of the robot controlling apparatus and/or the actuator 103 may operate to, in response to an error detection, stop the catheter and/or the bendable robot/device 104 from moving. By way of a few examples, one or more of the following may occur or may exist: (i) the indicator may be disposed in the input device/operating portion 105, in the robot controlling apparatus or the actuator 103, and/or in a display (e.g., the display 101-1, 1209, etc.) that is in communication with or is part of the apparatus/system 1000; (ii) the robot controlling apparatus and/or the actuator 103 may move along the slider and/or the rail 110 (e.g., in accordance with a control by the host controller 801); (iii) the instruction may include an instruction to move the robot controlling apparatus and/or the actuator 103 along the slider and/or the rail 110 and the processor or controller (e.g., the processor 100, 102, 1200, 1200′, any other processor discussed herein, etc.) of the robot controlling apparatus and/or the actuator 103 may operate to stop transferring, from that processor or controller of the robot controlling apparatus and/or the actuator 103 to the host controller 801, the information of the instruction corresponding to a movement of the robot controlling apparatus and/or the actuator 103 along the slider where the processor or controller (e.g., the processor 100, 102, 1200, 1200′, any other processor discussed herein, etc.) of the robot controlling apparatus and/or the actuator 103 detects the communication error (e.g., with the host controller 801); (iv) the processor or controller (e.g., the processor 100, 102, 1200, 1200′, any other processor discussed herein, etc.) of the robot controlling apparatus and/or the actuator 103 may disable the one or more motors (1-M, 144, etc.) to bend or from bending the catheter and/or the bendable robot/device 104 in a case where the processor or controller (e.g., the processor 100, 102, 1200, 1200′, any other processor discussed herein, etc.) of the robot controlling apparatus and/or the actuator 103 does not receive a predetermined response from the host controller 801 within a predetermined period or amount of time; and/or (v) the processor or controller (e.g., the processor 100, 102, 1200, 1200′, any other processor discussed herein, etc.) of the robot controlling apparatus and/or the actuator 103 may disable the one or more motors (1-M, 144, etc.) to bend or from bending the catheter and/or the bendable robot/device 104 in a case where the processor or controller (e.g., the processor 100, 102, 1200, 1200′, any other processor discussed herein, etc.) of the robot controlling apparatus and/or the actuator 103 does not receive any valid message from the host controller 801 within a predetermined period or amount of time.

In view of the above, one or more embodiments of the apparatus/system 1000 (and/or the robot controlling apparatus and/or the actuator 103) and/or the processor or controller (e.g., the processor 100, 102, 1200, 1200′, any other processor discussed herein, etc.) of the robot controlling apparatus and/or the actuator 103 may operate to include, control, and/or be in communication with a camera (e.g., the camera 106, the camera 180, another camera discussed herein, etc.) for imaging and/or monitoring a lumen 81 (e.g., an airway of a patient P, a vessel of a patient P, another lumen 81 of a patient P, a lumen in a target, sample, or object, etc.) where the camera may be connected to the host apparatus 800 and/or the host controller 801 of the host apparatus 800. The camera (e.g., the camera 106, the camera 180, etc.) may be disposed or inserted in a lumen of the catheter and/or the bendable robot/device 104. In one or more embodiments, an image captured by the camera (e.g., the camera 106, the camera 180, etc.) may be displayed on the display (e.g., the display 101-1, 1209, etc.), and/or the host controller 801 may keep the image captured by the camera being displayed, even in a case where the processor or controller (e.g., the processor 100, 102, 1200, 1200′, any other processor discussed herein, etc.) of the robot controlling apparatus and/or the actuator 103 may detect the communication error (e.g., with the host controller 801).

One or more of the aforementioned features may be used with a continuum robot and related features as disclosed in U.S. Pat. No. 11,882,365, issued on Jan. 23, 2024, the disclosure of which is incorporated by reference herein in its entirety. For example, at least one embodiment of a continuum robot apparatus configuration may be used to implement automatic correction of a direction to which a tool channel or a camera moves or is bent in a case where a displayed image is rotated. The continuum robot apparatus enables to keep a correspondence between a direction on a monitor (top, bottom, right or left of the monitor) and a direction the tool channel or the camera moves on the monitor according to a particular directional command (up, down, turn right or turn left) even if the displayed image is rotated. The continuum robot apparatus also may be used with any of the host and robot communication, error avoidance or removal communication, navigation planning, autonomous navigation, movement detection, and/or control features of the present disclosure.

The user may provide an operation input through an input element, and the continuum robot apparatus/system 1000, catheter/continuum robot/bendable robot/device 104, other apparatus discussed herein, etc. may receive information of the input element and one or more input/output devices, which may include, but are not limited to, a receiver, a transmitter, a speaker, a display, an imaging sensor, a user input device, which may include a keyboard, a keypad, a mouse, a position tracked stylus, a position tracked probe, a foot switch, a microphone, a camera, etc. A user may adjust various parameters of the robot apparatus/system 1000, catheter/continuum robot/bendable robot/device 104, other apparatus discussed herein, etc., such as the speed (e.g., rotational speed, translational speed, etc.), angle or plane, or other parameters.

The continuum robot apparatus/system 1000, catheter/continuum robot/bendable robot/device 104, other apparatus discussed herein, etc. may be interconnected with medical instruments or other devices, and may be controlled independently, externally, or remotely by a controller. In one or more embodiments of the present disclosure, one or more features of the continuum robot apparatus(es) as discussed in U.S. Prov. Pat. App. No. 63/742,346, filed on Jan. 6, 2025, which is incorporated by reference herein in its entirety, and one or more features of the continuum robot or catheter or probe apparatus/system 1000, catheter/continuum robot/bendable robot/device 104, any other apparatus or system discussed herein, etc. may be used in combination or alternatively to each other.

The memory may be used as a work memory or may include any memory discussed in the present disclosure. The storage stores software or computer instructions, and may be any type of storage, data storage 150, or other memory or storage discussed in the present disclosure. The CPU, which may include one or more processors, circuitry, or a combination thereof, executes the software developed in the memory (e.g., RAM 130 or any other memory discussed herein). The I/O interface operates to input information from the continuum robot apparatus/system 1000, catheter/continuum robot/bendable robot/device 104, other apparatus discussed herein, etc. to the controller(s) (e.g., the controller 100, the controller 102, any other controller or processor discussed herein, etc.) and to output information for displaying to the display 101-1 (or any other display discussed herein, such as, but not limited to, display 1209 discussed below).

The communication interface 1205 may be configured as a circuit or other device for communicating with components included in the apparatus/system 1000, catheter/continuum robot/bendable robot/device 104, etc., and with various external apparatuses connected to the apparatus via a network. For example, the communication interface 1205 may store information to be output in a transfer packet and may output the transfer packet to an external apparatus via the network by communication technology such as Transmission Control Protocol/Internet Protocol (TCP/IP). The apparatus may include a plurality of communication circuits according to a desired communication form.

The controller may be communicatively interconnected or interfaced with external device(s) including, for example, one or more data storages (e.g., the data storage 150, the SSD or storage drive 1207 discussed below, or any other storage discussed herein), one or more external user input/output devices, or the like. The controller may interface with other elements including, for example, one or more of an external storage, a display, a keyboard, a mouse, a sensor, a microphone, a speaker, a projector, a scanner, a display, an illumination device, etc.

The display may be a display device configured, for example, as a monitor, an LCD (liquid panel display), an LED display, an OLED (organic LED) display, a plasma display, an organic electro luminescence panel, or any other display discussed herein (including, but not limited to, displays 101-1, 101-2, 1209, etc.). Based on the control of the apparatus, a screen may be displayed on the display showing image(s), such as, but not limited to, one or more images being captured, captured images, captured moving images recorded on the storage unit, etc.

The components may be connected together by a BUS (or “Bus”) or other connection lines (e.g., connection line 1213) so that the components may communicate with each other. The bus transmits and receives data between these pieces of hardware connected together, or the bus transmits a command from the CPU (e.g., 100, 102, 1200, 1200′, 1201, etc.) to the other pieces of hardware. The components may be implemented by one or more physical devices that may be coupled to the CPU (e.g., 100, 102, 1200, 1200′, 1201, etc.) through a communication channel. For example, the controller may be implemented using circuitry in the form of ASIC (application specific integrated circuits) or other similar circuits as discussed herein. Alternatively, the controller may be implemented as a combination of hardware and software, where the software is loaded into a processor from a memory or over a network connection. Functionality of the controller may be stored on a storage medium, which may include, but is not limited to, RAM (random-access memory), magnetic or optical drive, diskette, cloud storage, etc.

The units described herein are exemplary and/or preferable modules for implementing processes described in the present disclosure. However, one or more embodiments of the present disclosure are not limited thereto. The term “unit”, as used herein, may generally refer to firmware, software, hardware, or other component, such as circuitry or the like, or any combination thereof, that is used to effectuate a purpose. The modules may be hardware units (such as circuitry, firmware, a field programmable gate array, a digital signal processor, an application specific integrated circuit, or the like) and/or software modules (such as a computer readable program, instructions stored in a memory or storage medium, etc.). The modules for implementing the various steps are not described exhaustively above. However, where there is a step of performing a certain process, there may be a corresponding functional module or unit (implemented by hardware and/or software) for implementing the same process. Technical solutions by all combinations of steps described and units corresponding to these steps are included in the present disclosure.

One or more host and robot communication, error avoidance or elimination/removal communication, navigation planning, autonomous navigation, movement detection, and/or control features of the present disclosure may be used with one or more image correction or adjustment features in one or more embodiments. One or more adjustments, corrections, or smoothing functions for a catheter or probe device and/or a continuum robot may adjust a path of one or more sections or portions of the catheter or probe device and/or the continuum robot (e.g., the continuum robot 104, the continuum robot device/system 1000, etc.), and one or more embodiments may make a corresponding adjustment or correction to an image view. For example, in one or more embodiments the medical tool may be a bronchoscope.

While one or more features of the present disclosure have been described with reference to exemplary embodiments, it is to be understood that the present disclosure is not limited to the disclosed exemplary embodiments. The scope of the claims is to be accorded the broadest interpretation so as to encompass all modifications, equivalent structures, and functions.

A computer, such as the console or computer 1200, 1200′, may perform any of the steps, processes, and/or techniques discussed herein for any apparatus, bronchoscope, robot, and/or system being manufactured or used, any of the embodiments shown in FIGS. 1-16, any other bronchoscope, robot, apparatus, or system discussed herein or included herewith, etc.

There are many ways to control a bronchoscope or robotic bronchoscope and/or a continuum robot or robotic device, perform imaging, host and robot communication, error avoidance or removal communication, navigation planning, autonomous navigation, movement detection, and/or control for a continuum robot, correct or adjust an image or a path/state (or one or more sections or portions of) a continuum robot (or other probe or catheter device or system), or perform any other measurement or process discussed herein, to perform continuum robot and/or bronchoscope method(s) or algorithm(s), and/or to control at least one bronchoscope and/or continuum robot device/apparatus/robot, system and/or storage medium, digital as well as analog. In at least one embodiment, a computer, such as the console or computer 1200, 1200′, may be dedicated to control and/or use continuum robot and/or bronchoscope devices, systems, methods, and/or storage mediums for use therewith described herein.

The one or more detectors, sensors, cameras, or other components of the bronchoscope, robotic bronchoscope, robot, continuum robot, apparatus, system, method, or storage medium embodiments (e.g. of the system 1000 of FIG. 1, or any other system discussed herein; of any bronchoscope, robot, apparatus, system, method, or storage medium discussed herein; etc.) may transmit the digital or analog signals to a processor or a computer such as, but not limited to, an image processor or display controller 100, a controller 102, a CPU 120, a processor or computer 1200, 1200′, host controller 801, processor of the robot controlling apparatus and/or actuator 103 (see e.g., at least FIGS. 1-8B and 10-16), a combination thereof, any other processor(s) discussed herein, etc. The image processor may be a dedicated image processor or a general purpose processor that is configured to process images. In at least one embodiment, the computer 1200, 1200′ may be used in place of, or in addition to, the image processor or display controller 100 and/or the controller 102 (or any other processor or controller discussed herein, such as, but not limited to, the host controller 801, the processor or controller of the robot controlling apparatus and/or the actuator 103, the CPU 120, etc.). In an alternative embodiment, the image processor may include an ADC and receive analog signals from the one or more detectors or sensors of the bronchoscopes, robots, apparatuses, systems (e.g., apparatus/system 1000 (or any other system discussed herein)), methods, storage mediums, etc. The image processor may include one or more of a CPU, DSP, FPGA, ASIC, or some other processing circuitry. The image processor may include memory for storing image, data, and instructions. The image processor may generate one or more images based on the information provided by the one or more detectors, sensors, or cameras. A computer or processor discussed herein, such as, but not limited to, a processor of the devices, apparatuses, bronchoscopes, or systems of FIGS. 1-16, the computer 1200, the computer 1200′, the image processor, etc. may also include one or more components further discussed herein below (see e.g., FIGS. 10-16).

Electrical analog signals obtained from the output of the apparatus/system 1000 or the components thereof, and/or from the devices, bronchoscopes, apparatuses, or systems of FIGS. 1-16, may be converted to digital signals to be analyzed with a computer, such as, but not limited to, the computers or controllers 100, 102 of FIG. 1, the computer 1200, 1200′, etc.

There are many ways to perform imaging, control a bronchoscope or robotic bronchoscope, perform navigation planning, host and robot communication, error avoidance or removal communication, autonomous navigation, movement detection, and/or control for a continuum robot, perform bronchoscope or robotic bronchoscope method(s) or algorithm(s), control at least bronchoscope or robotic device/apparatus/bronchoscope, system, and/or storage medium, correct or adjust an image, correct, adjust, or smooth a path/state (or section or portion) of a continuum robot, or perform any other measurement or process discussed herein, to perform continuum robot method(s) or algorithm(s), and/or to control at least one continuum robot device/apparatus, system and/or storage medium, digital as well as analog. By way of a further example, in at least one embodiment, a computer, such as the computer or controllers 100, 102 of FIG. 1, the console or computer 1200, 1200′, etc., may be dedicated to the host and robot communication (see e.g., at least host controller/processor 801 and the processor or controller of the robot controlling apparatus and/or the actuator 103 of at least FIG. 8B), error avoidance or removal communication, autonomous navigation/planning/control and the monitoring of the bronchoscopes, robotic bronchoscopes, devices, systems, methods, and/or storage mediums and/or of continuum robot devices, systems, methods and/or storage mediums described herein.

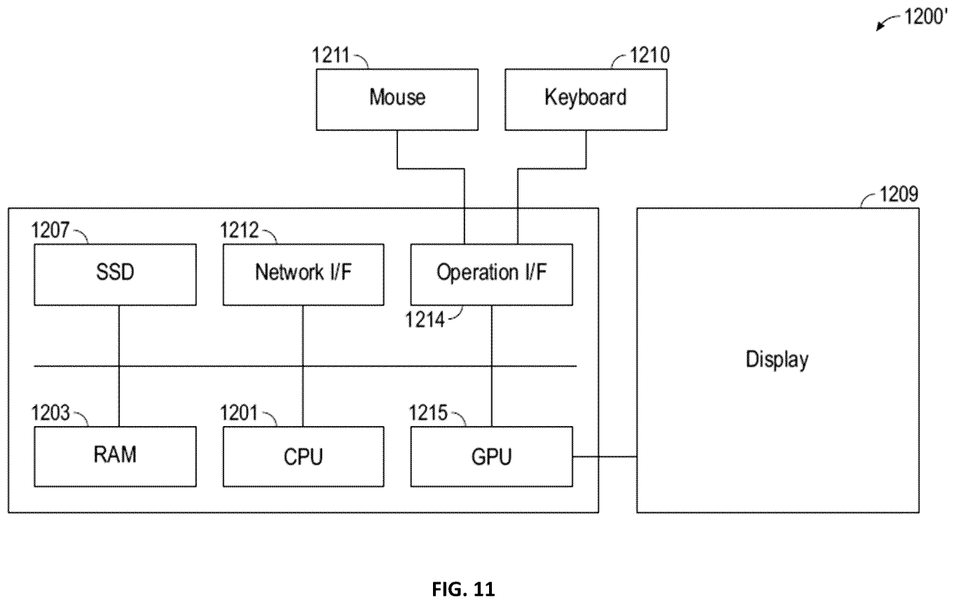

The electric signals used for imaging may be sent to one or more processors, such as, but not limited to, the processors or controllers 100, 102 of FIGS. 1-6, a computer 1200 (see e.g., FIG. 10), a computer 1200′ (see e.g., FIG. 11), etc. as discussed further below, via cable(s) or wire(s), such as, but not limited to, the cable(s) or wire(s) 113 (see FIG. 1). Additionally or alternatively, the computers or processors discussed herein are interchangeable, and may operate to perform any of the feature(s) and method(s) discussed herein.

Various components of a computer or processor apparatus/system 1200 (see e.g., the console or computer 1200 as may be used as one embodiment example of the computer, processor, or controllers 100, 102 shown in FIG. 1) are provided in FIG. 10. A computer system/processor/controller 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., as shown in FIG. 10). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a robot device, apparatus, bronchoscope or robotic bronchoscope, or system using same, and/or a continuum robot device or system using same, such as, but not limited to, the apparatus/system 1000, the apparatus/system, the devices/systems of FIGS. 1-9B, and/or other systems/apparatuses discussed herein above, via one or more lines 1213), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The computer apparatus/system/processor/controller 1200 may include one or more additional processors in addition to CPU 1201, and such processors, including the CPU 1201, may be used for controlling and/or manufacturing a device, system or storage medium for use with same or for use with any continuum robot, bronchoscope, or robotic bronchoscope technique(s), and/or use with imaging, host and robot communication, error avoidance or removal communication, navigation planning, autonomous navigation, movement detection, and/or control technique(s) discussed herein. The processor/controller/apparatus/system 1200 may further include one or more processors connected via a network connection (e.g., via network 1206). The CPU 1201 and any additional processor being used by the system 1200 may be located in the same telecom network or in different telecom networks (e.g., performing, manufacturing, controlling, calculation, and/or using technique(s) may be controlled remotely).

The I/O or communication interface 1205 provides communication interfaces to input and output devices, which may include the one or more of the aforementioned components of any of the bronchoscopes, robotic bronchoscopes, apparatuses, devices, and/or systems discussed herein (e.g., the controller 100, the controller 102, the displays 101-1, 101-2, the actuator 103, the continuum device/catheter/bendable robot/device 104, the operating portion or controller/input device 105, the camera 106, the EM tracking sensor 106 (when used), the position detector 107, the rail 110, the camera 180, etc.), a microphone, a communication cable and a network (either wired or wireless), a keyboard 1210, a mouse (see e.g., the mouse 1211 as shown in FIG. 11), a touch screen or screen 1209, a light pen and so on. The communication interface of the computer 1200 may connect to other components discussed herein via line 113 (as diagrammatically shown in FIG. 10). The monitor interface or screen 1209 provides communication interfaces thereto. In one or more embodiments, the camera 106 is used alone or in combination with the EM sensor 106, and the position detector 107 may be optional (e.g., may be used along with the EM sensor 106). The camera 180 may be used in addition in one or more embodiments.

Any methods and/or data of the present disclosure, such as, but not limited to, the methods for using/guiding and/or controlling a bronchoscope, robotic bronchoscope, continuum robot, or catheter device, apparatus, system, or storage medium for use with same and/or method(s) for imaging, host and robot communication, error avoidance or removal communication, performing tissue, lesion, or sample characterization or analysis, performing diagnosis, planning and/or examination, for controlling a bronchoscope or robotic bronchoscope, device/apparatus, or system, for performing navigation planning, autonomous navigation, movement detection, and/or control technique(s), for performing adjustment or smoothing techniques (e.g., to a path of, to a pose or position of, to a state of, or to one or more sections or portions of, a continuum robot, a catheter or a probe), and/or for performing imaging and/or image correction or adjustment technique(s), (or any other technique(s)) as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”), a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 11), SRAM, etc.), an optional combination thereof, a server/database, a neural network (or other AI architecture/structure/models) etc. may be used to cause a processor, such as, the processor or CPU 1201 of the aforementioned computer apparatus/system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) may also be realized by a computer and/or neural network (or other AI architecture/structure/models) of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, bronchoscopes or robotic bronchoscopes, devices, systems, and computer-readable storage mediums related to the processors, such as, but not limited to, the processor(s) of the aforementioned computer 1200, 1200′, the controller 100, the controller 102, the host controller 801, the processor or controller of the robot controlling apparatus and/or the actuator 103, any other processor or controller discussed herein, etc., as described herein may be achieved utilizing suitable hardware, such as that illustrated in the figures. Functionality of one or more aspects of the present disclosure may be achieved utilizing suitable hardware, such as that illustrated in FIG. 10. Such hardware may be implemented utilizing any of the known technologies, such as standard digital circuitry, any of the known processors that are operable to execute software and/or firmware programs, any neural networks (and/or any other artificial intelligence (AI) structure/architecture/models/etc. that may be used to perform any of the technique(s) discussed herein) one or more programmable digital devices or systems, such as programmable read only memories (PROMs), programmable array logic devices (PALs), etc. The CPU 1201 (as shown in FIG. 10 or FIG. 11, and/or which may be included in the computer, processor, controller and/or CPU 120 of FIGS. 1-9B and/or of any of the other figures or embodiments discussed herein), and/or the CPU 120 may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 100, 102, 120, 801, 1200, 1200′, any other computer or processor discussed herein, etc.) may include the aforementioned CPU structure, or may be connected to CPU structure for communication therewith.

As aforementioned, hardware structure of an alternative embodiment of a computer or console 1200′ is shown in FIG. 11. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid-state drive (SSD) 1207. Preferably, the computer or console 1200′ includes a display 1209 (and/or the displays 101-1, 101-2, any other display(s) discussed herein, etc.). The computer 1200′ may connect with one or more components of a system (e.g., the systems/apparatuses/bronchoscopes/robotic bronchoscopes discussed herein; the systems/apparatuses/bronchoscopes/robotic bronchoscopes and/or any other device, apparatus, system, etc. of any of the figures included herewith (e.g., the systems/apparatuses of FIGS. 1-9B, 10-16, etc.)) via the operation interface 1214 or the network interface 1212. The operation interface 1214 is connected with an operation unit such as a mouse device 1211, a keyboard 1210 or a touch panel device. The computer 1200′ may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 1200, 1200′, etc.

At least one computer program is stored in the SSD 1207 (or any other storage device/drive discussed herein), and the CPU 1201 loads the at least one program onto the RAM 1203, and executes the instructions in the at least one program to perform process(es) described herein, and the basic input, output, calculation, memory writing, and memory reading processes.

The computer, such as the computer 1200, 1200′, the computer, processors, and/or controllers of FIGS. 1-9B and/or of any other figure(s) included herewith, etc., communicates with the one or more components of the apparatuses/systems/bronchoscopes/robotic bronchoscopes/robots of FIGS. 1-9B, of any other figure(s) included herewith, etc. and/or of any other apparatuses/systems/bronchoscopes/robotic bronchoscopes/robots/etc. discussed herein, to perform imaging, and reconstruct an image from the acquired intensity data. The monitor or display 1209 displays the reconstructed image, and the monitor or display 1209 may display other information about the imaging condition or about an object, target, or sample to be imaged. The monitor 1209 also provides a graphical user interface for a user to operate a system, for example when performing CT, MRI, or other imaging technique(s), including, but not limited to, controlling continuum robots/bronchoscopes/robotic bronchoscopes/devices/systems, and/or performing imaging, host and robot communication, error avoidance or removal communication, navigation planning, autonomous navigation, movement detection, and/or control technique(s). An operation signal is input from the operation unit/operating portion/input device 105 (e.g., such as, but not limited to, a joystick, a mouse device 1211, a keyboard 1210, a touch panel device, etc.) into the operation interface 1214 in the computer 1200′, and corresponding to the operation signal the computer 1200′ instructs the apparatus/bronchoscope/robotic bronchoscope/system (e.g., the apparatus/system 1000, the apparatus/system, the systems/apparatuses of FIGS. 1-9B, any other system/apparatus discussed herein, any of the apparatus(es)/bronchoscope(s)/robotic bronchoscope(s)/system(s) discussed herein, etc.) to start or end the imaging, and/or to start or end bronchoscope/robotic bronchoscope/device/system/continuum robot control(s) and/or performance of imaging, host and robot communication, error avoidance or removal communication, correction, adjustment, and/or smoothing technique(s). The camera or imaging device may have interfaces to communicate with the computers 1200, 1200′ (or any other computer or processor discussed herein) to send and receive the status information and the control signals.

As aforementioned techniques of the present disclosure may be performed using artificial intelligence structure(s), such as, but not limited to, residual networks, neural networks, convolutional neural networks, GANs, cGANs, etc. In one or more embodiments, other types of AI structure(s) and/or network(s) may be used. The below discussed network/structure examples are illustrative only, and any of the features of the present disclosure may be used with any AI structure or network, including AI networks that are less complex than the network structures discussed below (e.g., including such structure as show in FIGS. 10-16).

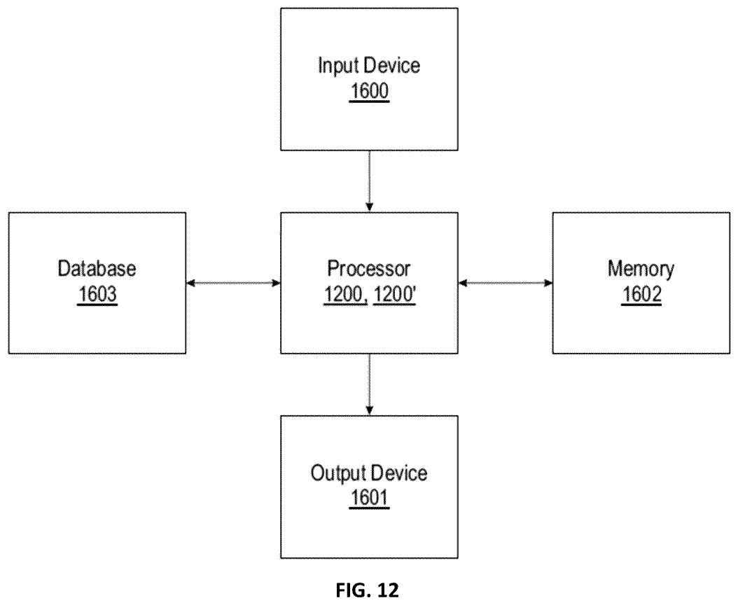

As shown in FIG. 12, one or more processors or computers 1200, 1200′ (or any other processor discussed herein) may be part of a system in which the one or more processors or computers 1200, 1200′ (or any other processor discussed herein) communicate with other devices (e.g., a database 1603, a memory 1602 (which may be used with or replaced by any other type of memory discussed herein or known to those skilled in the art), an input device 1600 (which may be any input device discussed herein or known to those skilled in the art, such as, but not limited to the input device 105, a joystick, the mouse 1211, the keyboard 1210, a touch screen, etc.), an output device 1601, etc.). In one or more embodiments, one or more models may have been trained previously and stored in one or more locations, such as, but not limited to, the memory 1602, the database 1603, etc. In one or more embodiments, it is possible that one or more models and/or data discussed herein (e.g., training data, testing data, validation data, imaging data, etc.) may be input or loaded via a device, such as the input device 1600. In one or more embodiments, a user may employ an input device 1600 (which may be a separate computer or processor, a keyboard such as the keyboard 1210, a mouse such as the mouse 1211, a microphone, a screen or display 1209 (e.g., a touch screen or display), or any other input device known to those skilled in the art or discussed herein). In one or more system embodiments, an input device 1600 (or any other input device/operating portion discussed herein, such as, but not limited to, the input device/operating portion 105 (which may be a joystick, a mouse device 1211, a keyboard 1210, a touch panel device, any other input device/operating portion discussed herein or known to those skilled in the art, etc.)) may not be used (e.g., where user interaction is eliminated by one or more artificial intelligence features discussed herein). In one or more system embodiments, the output device 1601 may receive one or more outputs discussed herein to perform coregistration, navigation planning, autonomous navigation, movement detection, control, and/or any other process discussed herein. In one or more system embodiments, the database 1603 and/or the memory 1602 may have outputted information (e.g., trained model(s), detected marker information, image data, test data, validation data, training data, coregistration result(s), segmentation model information, object detection/regression model information, combination model information, etc.) stored therein. That said, one or more embodiments may include several types of data stores, memory, storage media, etc. as discussed above, and such storage media, memory, data stores, etc. may be stored locally or remotely.

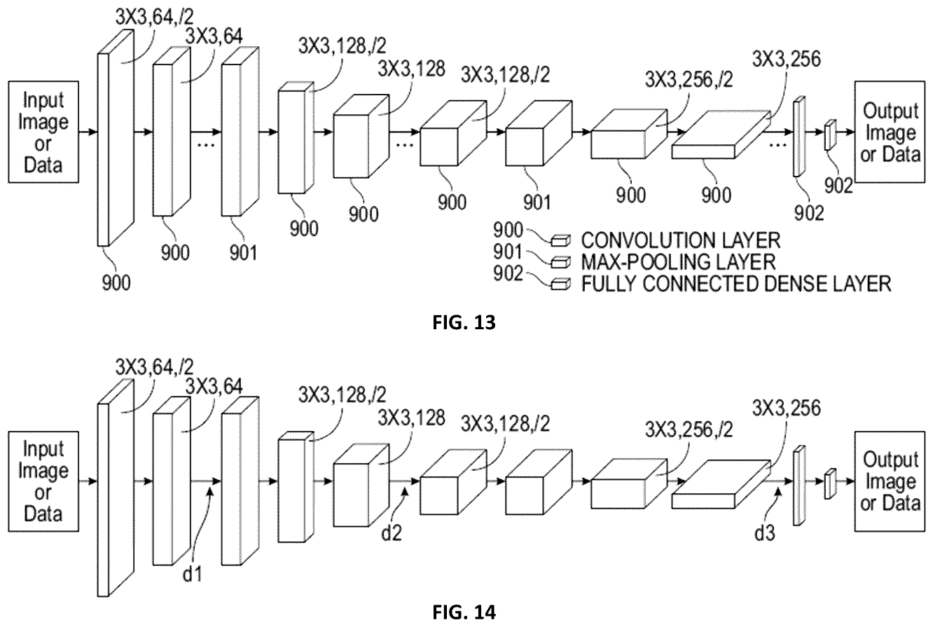

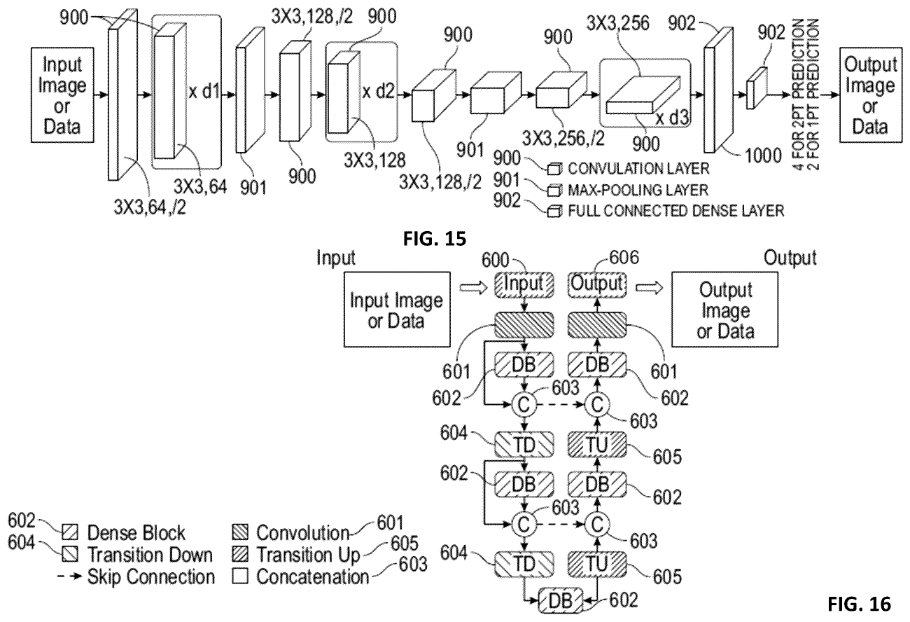

For regression model(s), the input may be the entire image frame(s), and the output may be the centroid coordinates of a target, an octagon, circle or other geometric shape used or discussed herein, one or more airways, and/or coordinates of a portion of a catheter or probe. As shown diagrammatically in FIGS. 13-15, an example of an input image on the left side of FIGS. 13-15 and a corresponding output image on the right side of FIGS. 13-15 are illustrated for regression model(s). At least one architecture of a regression model is shown in FIG. 13. In at least the embodiment of FIG. 13, the regression model may use a combination of one or more convolution layers 900, one or more max-pooling layers 901, and one or more fully connected dense layers 902. While not limited to the Kernel size, Width/Number of filters (output size), and Stride sizes shown for each layer (e.g., in the left convolution layer of FIG. 13, the Kernel size is “3×3”, the Width/# of filters (output size) is “64”, and the Stride size is “2”). Another hyper-parameter search with a fixed optimizer and with a different width may be performed, and at least one embodiment example of a model architecture for a convolutional neural network for this scenario is shown in FIG. 14. One or more embodiments may use one or more features for a regression model as discussed in “Deep Residual Learning for Image Recognition” to Kaiming He, et al., Microsoft Research, Dec. 10, 2015 (https://arxiv.org/pdf/1512.03385.pdf), which is incorporated by reference herein in its entirety. FIG. 15 shows at least a further embodiment example of a created architecture of/for a regression model(s).

Since the output from a segmentation model, in one or more embodiments, is a “probability” of each pixel that may be categorized as a target or as an estimate (incorrect) or actual (correct) match, post-processing after prediction via the trained segmentation model may be developed to better define, determine, or locate the final coordinate of catheter location and/or determine the navigation planning, autonomous navigation, movement detection, and/or control status of the catheter or continuum robot. One or more embodiments of a semantic segmentation model may be performed using the One-Hundred Layers Tiramisu method discussed in “The One Hundred Layers Tiramisu: Fully Convolutional DenseNets for Semantic Segmentation” to Simon Jégou, et al., Montreal Institute for Learning Algorithms, published Oct. 31, 2017 (https://arxiv.org/pdf/1611.09326.pdf), which is incorporated by reference herein in its entirety. A segmentation model may be used in one or more embodiment, for example, as shown in FIG. 16. At least one embodiment may utilize an input 600 as shown to obtain an output 606 of at least one embodiment of a segmentation model method. For example, by applying the One-Hundred Layers Tiramisu method(s), one or more features, such as, but not limited to, convolution 601, concatenation 603, transition up 605, transition down 604, dense block 602, etc., may be employed by slicing the training data set. While not limited to only or by only these embodiment examples, in one or more embodiments, a slicing size may be one or more of the following: 100×100, 224×224, 512×512. A batch size (of images in a batch) may be one or more of the following: 2, 4, 8, 16, and, from the one or more experiments performed, a bigger batch size typically performs better (e.g., with greater accuracy). In one or more embodiments, 16 images/batch may be used. The optimization of all of these hyper-parameters depends on the size of the available data set as well as the available computer/computing resources; thus, once more data is available, different hyper-parameter values may be chosen. Additionally, in one or more embodiments, steps/epoch may be 100, and the epochs may be greater than (>) 1000. In one or more embodiments, a convolutional autoencoder (CAE) may be used.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Pat. No. 11,882,365, issued on Jan. 23, 2024, the disclosure of which is incorporated by reference herein in its entirety. Such endoscope devices, systems, methods, and/or storage mediums are disclosed in at least: U.S. Pat. Pub. No. 2022/0202502 A1, published on Jun. 30, 2022, the disclosure of which is incorporated by reference herein in its entirety; and U.S. Pat. Pub. No. 2022/0202274 A1, published on Jun. 30, 2022, the disclosure of which is incorporated by reference herein in its entirety. Any of the features of the present disclosure may be used in combination with any of the features as discussed in U.S. Pat. Pub. No. 2024/0112407 A1, published Apr. 4, 2024, the disclosure of which is incorporated by reference herein in its entirety. Any of the features of the present disclosure may be used in combination with any of the features as discussed in U.S. Pat. Pub. No. 2023/0131269, published on Apr. 26, 2023, the disclosure of which is incorporated by reference herein in its entirety.

An imaging apparatus or system, such as, but not limited to, a robotic bronchoscope and/or imaging devices or systems, discussed herein may have or include three bendable sections. The visualization technique(s) and methods discussed herein may be used with one or more imaging apparatuses, systems, methods, or storage mediums of U.S. Pat. Pub. No. 2024/0112407 A1, published Apr. 4, 2024, the disclosure of which is incorporated by reference herein in its entirety.

Further, the present disclosure and/or one or more components of devices, systems, and storage mediums, and/or methods, thereof also may be used in conjunction with continuum robotic systems and catheters, such as, but not limited to, those described in U.S. Patent Publication Nos. 2019/0105468; 2021/0369085; 2020/0375682; 2021/0121162; 2021/0121051; and 2022-0040450, each of which patents and/or patent publications are incorporated by reference herein in their entireties.

The present disclosure and/or one or more components of devices, systems, and storage mediums, and/or methods, thereof also may be used in conjunction with autonomous robot devices, systems, methods, and/or storage mediums and/or with endoscope devices, systems, methods, and/or storage mediums. Such continuum robot devices, systems, methods, and/or storage mediums are disclosed in at least: PCT/US2024/025546, filed on Apr. 19, 2024, which is incorporated by reference herein in its entirety.

The present disclosure and/or one or more parts of devices, systems, and storage mediums, and/or methods, thereof also may be used in conjunction with autonomous robot devices, systems, methods, and/or storage mediums and/or with endoscope devices, systems, methods, and/or storage mediums, and/or other features that may be used with same, such as, but not limited to, any of the features disclosed in at least: International Patent Application No. PCT/US2024/037935, filed Jul. 12, 2024, the disclosure of which is incorporated by reference herein in its entirety; International Patent Application No. PCT/US2024/037924, the disclosure of which is incorporated by reference herein in its entirety; and International Patent Application No. PCT/US2024/037930, the disclosure of which is incorporated by reference herein in its entirety.

Although the disclosure herein has been described with reference to particular features and/or embodiments, it is to be understood that these features and/or embodiments are merely illustrative of the principles and applications of the present disclosure (and are not limited thereto), and the scope of the present disclosure is not limited to the disclosed features and/or embodiments. It is therefore to be understood that numerous modifications may be made to the illustrative features and/or embodiments and that other arrangements may be devised without departing from the spirit and scope of the present disclosure. Indeed, the present disclosure encompasses and includes any combination of any of the feature(s) and/or embodiment(s) (or component(s) thereof) discussed herein. The scope of the following claims is to be accorded the broadest interpretation so as to encompass all such modifications, equivalent structures, and functions.

1. A robot controlling apparatus or actuator comprising:
one or more processors that operate to:
check a status of one or more communications of the robot controlling apparatus or actuator with a host processor of a host apparatus;
determine whether an error is detected in the one or more communications with the host processor; and
in a case where the error is detected in the one or more communications with the host processor, stop a bendable robot or catheter from moving, bending, or changing a state of the bendable robot or catheter.

2. The robot controlling apparatus or actuator of claim 1, wherein:
(i) the robot controlling apparatus or actuator is connected to or in communication with an input device via a first interface of the robot controlling apparatus or actuator, the input device operating to transmit one or more instructions to the one or more processors of the robot controlling apparatus or actuator where the one or more instructions operate to move, control, bend, or change the state of the bendable robot or catheter; and
(ii) the robot controlling apparatus or actuator is connected to or in communication with the host apparatus via a second interface of the robot controlling apparatus or actuator such that the host processor of the host apparatus and the one or more processors of the robot controlling apparatus or actuator transmit the one or more communications and/or transmit the one or more instructions from the input device to the host processor of the host apparatus.

3. The robot controlling apparatus or actuator of claim 2, wherein one or more of the following:
(i) the host processor of the host apparatus operates to generate a Graphical User Interface (GUI) based on information of the one or more instructions from the input device and/or to display posture, position, orientation, bending, or other state information of the bendable robot or catheter;
(ii) the host processor of the host apparatus operates to generate a Graphical User Interface (GUI) based on information of the one or more instructions from the input device and/or to display current posture, position, orientation, bending, or other state information of the bendable robot or catheter;
(iii) the robot controlling apparatus or actuator is connected to or in communication with a display that operates to display a Graphical User Interface (GUI) to display posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately;
(iv) the robot controlling apparatus or actuator is connected to or in communication with a display that operates to display a Graphical User Interface (GUI) to display current posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately; and/or
(v) in the case where the error is detected in the one or more communications with the host processor, the robot controlling apparatus or actuator and/or the bendable robot or catheter is/are put in a safety shutdown mode to avoid misleading a user of the robot controlling apparatus or actuator and/or the bendable robot or catheter since the user cannot see result(s) of one or more instructions or actions of the user.

4. The robot controlling apparatus or actuator of claim 2, wherein, in the case where the robot controlling apparatus or actuator is connected to or in communication with the host apparatus via the second interface of the robot controlling apparatus or actuator, one or more of the following:
(i) the one or more processors of the robot controlling apparatus or actuator further operate to send one or more of the following to the host processor of the host apparatus: state information of the bendable robot or catheter; state information of the bendable robot or catheter indicating whether the bendable robot or catheter is connected to the robot controlling apparatus or actuator or not; state information or navigation information of the bendable robot or catheter as a tip of the bendable robot or catheter and/or a linear stage connected to the bendable robot or catheter is or are manipulated, controlled, or moved; one or more requests from a user of the robot controlling apparatus or actuator or change the state of the bendable robot or catheter; one or more requests from the user to take one or more images or snapshots using a camera of the bendable robot or catheter; one or more requests from the user to rotate the one or more images or snapshots of the camera of the bendable robot or catheter; information for a warning indicating the error; information for a warning, failure, or error related to a voltage being out of a set or predetermined range, a temperature being out of a set or predetermined range, and/or an emergency stop; version information and parameter values; and/or information automatically sent to the host processor or information manually sent to the host processor in response to a request for the information;
(ii) the host processor of the host apparatus operates to send one or more of the following to the one or more processors of the robot controlling apparatus or actuator: one or more requests for state, version, voltage, temperature, and/or parameter information; one or more requests to home or to return to a default, predetermined, or rest position/state a linear stage being used to move the bendable robot or catheter; one or more requests to zero or reset one or more motors of the robot controlling apparatus or actuator that operate to change the state of the bendable robot or catheter; one or more requests to change the state of the bendable robot or catheter and/or the linear stage; information for a test or normal joystick mode; error report(s); a request for shutdown or stopping of the bendable robot or catheter and/or the robot controlling apparatus or actuator; and/or Electromagnetic Compatibility (EMC) testing parameters and operations; and/or
(iii) the host processor of the host apparatus operates to perform one or more of the following: generation of a Graphical User Interface (GUI) based on information of the one or more instructions from the input device and/or to display current posture, position, orientation, bending, or other state information of the bendable robot or catheter; display of the GUI on a display; one or more pre-procedure operations related to login and/or entering case information; one or more procedure setup operations; one or more procedure operations related to displaying video from a camera of the bendable robot or catheter, showing the state or statue of the bendable robot or catheter, showing the state or statue of the bendable robot or catheter including one or more forces affecting one or more drive wires of the bendable robot or catheter, showing one or more warnings related to displaying the error information, and/or showing additional video feed(s); post-procedure operations related to reviewing cases and/or exporting the cases to a memory for storage; one or more management operations related to adding or removing user(s) of the robot controlling apparatus or actuator, the input device, and/or of the host apparatus, and/or related to handling one or more settings for each of the user(s); and/or writing or storing a log to a disk or to the memory.

5. The robot controlling apparatus or actuator of claim 2, further comprising or being in communication with one or more indicators that operate to indicate that the error is happening or has happened, in response to the detection of the error in the one or more communications with the host processor, wherein one or more of the following:
(i) the one or more indicators independently or automatically signal that the error is happening or has happened, or the one or more indicators independently or automatically signal that the error is happening or has happened to a user of the robot controlling apparatus or actuator and/or the input device;
(ii) the one or more indicators are disposed in or on one or more of the following: the robot controlling apparatus or actuator, the input device, the host apparatus, and/or a display that operates to display a Graphical User Interface (GUI); and/or
(iii) the bendable robot or catheter, the input device, the host apparatus, and/or the robot controlling apparatus or actuator operates to independently signal that the error is happening or has happened via the one or more indicators as a primary or secondary signal of the error.

6. The robot controlling apparatus or actuator of claim 1, further comprising one or more motors, wherein one or more of the following:
(i) the one or more processors of the robot controlling apparatus or actuator further operate to control the one or more motors to move, control, bend, or change the state of the bendable robot or catheter;
(ii) the robot controlling apparatus or actuator is connected to or in communication with the bendable robot or catheter such that the one or more processors and the one or more motors of the robot controlling apparatus operate to move, control, bend, or change the state of the bendable robot or catheter; and/or
(iii) in the case where the error is detected in the one or more communications with the host processor, the one or more processors of the robot controlling apparatus or actuator further operate to restrict, disable, or stop the one or more motors from moving, controlling, bending, or changing the state of the bendable robot or catheter.

7. The robot controlling apparatus or actuator of claim 6, wherein the one or more processors of the robot controlling apparatus or actuator further operate to restrict, disable, or stop the one or more motors from moving, controlling, bending, or changing the state of the bendable robot or catheter in a case where the one or more processors of the robot controlling apparatus or actuator do not receive a predetermined response and/or a valid message from the host processor of the host apparatus within a predetermined period or amount of time, the predetermined response and/or the valid message including one or more of the following: a query version string; a request to reset the bendable robot or catheter; a request to run power-on test(s); a request or query for a cyclic redundancy check (CRC) and/or for an on or off status of the CRC; a parameter(s) query; a request to query, notify, or set a failure string; a request to query, notify, or set a warning string; a request to notify or log a string or log string to an application log; a request to shut down the bendable robot or catheter and/or the robot controlling apparatus or actuator; a request to start a new procedure; a request to power cycle; a request for a query of complete status; a request to query, notify or set a mode, a disconnect mode, a disabled mode, a halt mod, a tip control mode, a manual mode, a pause mode, a relax mode, a park mode, a Follow-the-Leader (FTL) mode, a reverse FTL mode, a targeting mode, and/or an error mode; a request to query or notify an emergency stop or E-stop, or to obtain an ok or tripped status of the emergency stop; a request to establish a home, zero, or default position for the bendable robot or catheter for one or more or all motors or drive wires of the robot controlling apparatus or actuator; a request to query or notify a connected or a disconnected status of the bendable robot or catheter; and/or a request for a wire test of the bendable robot or catheter.

8. The robot controlling apparatus or actuator of claim 1, wherein one or more of the following:
(i) the one or more processors of the robot controlling apparatus or actuator further operate to determine whether the error is detected in the one or more communications with the host processor by using or communicating with one or more of the following: a driver or an operating system driver of the robot controlling apparatus or actuator, firmware of the robot controlling apparatus or actuator, software of the robot controlling apparatus or actuator, a robot state from the host processor of the host apparatus, and/or a serial communications and protocol manager of the host processor of the host apparatus;
(ii) the one or more processors of the robot controlling apparatus or actuator further operate to control an operation mode of the bendable robot or catheter to be in a relaxed mode in the case where the error is detected in the one or more communications with the host processor, or the one or more processors of the robot controlling apparatus or actuator further operate to control an operation mode of the bendable robot or catheter to be in a relaxed mode in the case where the error is detected in the one or more communications with the host processor where the bendable robot or catheter bends or changes the state of the bendable robot or catheter based on or in accordance with an external force so that the bendable robot or catheter conforms to a lumen of an object, target, or sample in a case where the bendable robot or catheter is removed from the lumen of the object, target, or sample; and/or
(iii) the one or more processors of the robot controlling apparatus or actuator further operate to control an operation mode of the bendable robot or catheter to be in a relaxed mode in the case where the error is detected in the one or more communications with the host processor such that the relaxed mode operates as a safety mode where the bendable robot or catheter is removed from an object, target, or sample.

9. The robot controlling apparatus or actuator of claim 1, further comprising a slider, a rail, and/or a translational stage that operates to move the robot controlling apparatus or actuator and/or the bendable robot or catheter to provide depth control or remember a path along or into a lumen of a target, sample, or object, wherein one or more of the following:
(i) the one or more processors of the robot controlling apparatus or actuator and/or the host processor further operate(s) to provide the depth control or to remember the path and to move the bendable robot or catheter using one or more of the following: a Follow-The-Leader (FTL) algorithm or process to move one or more sections of the bendable robot or catheter along the path; and/or a reverse Follow-The-Leader (rFTL) algorithm or process to reverse the path exactly or to re-orient the one or more sections of the bendable robot or catheter in a manner in which there is less impedance in the bending used to align the bendable robot or catheter with the lumen of the target, sample, or object; and/or
(ii) the one or more processors of the robot controlling apparatus or actuator and/or the host processor further operate(s) to, in the case where the error is detected in the one or more communications with the host processor, stop the movement of the robot controlling apparatus or actuator and/or the bendable robot or catheter via the slider, the rail, and/or the translational stage; or the one or more processors of the robot controlling apparatus and/or the host processor further operate(s) to, in the case where the error is detected in the one or more communications with the host processor, stop the movement of the robot controlling apparatus or actuator and/or the bendable robot or catheter via the slider, the rail, and/or the translational stage such that a user of the robot controlling apparatus or actuator, of the host apparatus, and/or of the bendable robot or catheter is stopped or prevent from driving the bendable robot or catheter further into the lumen with no visibility or information of action(s) of the user.

10. The robot controlling apparatus or actuator of claim 9, wherein the one or more processors of the robot controlling apparatus or actuator and/or the host processor further operate to one or more of the following:
(i) estimate or determine a depth of the depth control and/or estimate or determine the path to the target, sample, or object;
(ii) estimate or determine a depth of the depth control and/or estimate or determine the path to the target, sample, or object using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture; and/or
(iii) use a neural network, a convolutional neural network, a generative adversarial network (GAN), a consistent generative adversarial network (cGAN), a three cycle-consistent generative adversarial network (3cGAN), recurrent neural networks, and/or any other AI architecture to one or more of: use or evaluate an estimated or determined depth or path, identify one or more target points in the lumen or along the path, evaluate the accuracy of the identified one or more target points, plan the navigation of the bendable robot or catheter, autonomously move the bendable robot or catheter to the one or more target points, take one or more images, frames, or views using a camera of the bendable robot or catheter, and/or display the one or more target points on one of the one or more images, frames, or views on a display or indicate on the display or via an indicator on the display or via a light or indicator on or in communication with the bendable robot or catheter that the navigation has been planned or determined.

11. The robot controlling apparatus or actuator of claim 1, further comprising a camera that operates to be inserted into a lumen or be disposed at a distal end of the bendable robot or catheter to obtain one or more images or a live video feed for reference and/or for navigation in a lumen of an object, target, or sample, wherein one or more of the following:
(i) the one or more images or the live video feed is displayed on a display; and/or
(ii) the host processor further operates to keep the one or more images or the live video feed being displayed even in the case where the error is detected to allow for operation, movement, and/or removable of the bendable robot or catheter.

12. The robot controlling apparatus or actuator of claim 1, wherein one or more of the following:
(i) the bendable robot or catheter includes a distal bending section or portion, wherein the distal bending section or portion is commanded or instructed automatically or based on an input of a user of the bendable robot or catheter;
(ii) the bendable robot or catheter includes a plurality of bending sections or portions including a distal or most distal bending portion or section and the rest of the plurality of the bending sections or portions, where the plurality of bending sections or portions each include driving wires that operate to bend a respective section or portion of the plurality of sections or portions, and where the driving wires are connected to the robot controlling apparatus or actuator or to respective one or more motors of the robot controlling apparatus or actuator so that the robot controlling apparatus or actuator operates to bend one or more of the plurality of bending sections or portions using the driving wires; and/or
(iii) the robot controlling apparatus further comprises a slider, a rail, and/or a translational stage, and the one or more processors further operate to instruct or command the forward motion, or the motion in a set or predetermined direction, of the slider, the rail, the translational stage, and/or the bendable robot or catheter automatically or autonomously and/or based on an input of a user of the bendable robot or catheter.

13. A method for performing error detection or error removal for a robot controlling apparatus or actuator, the method comprising:
checking a status of one or more communications of one or more processors of the robot controlling apparatus or actuator with a host processor of a host apparatus;
determining whether an error is detected in the one or more communications with the host processor; and
in a case where the error is detected in the one or more communications with the host processor, stopping a bendable robot or catheter from moving, bending, or changing a state of the bendable robot or catheter.

14. The method of claim 13, further comprising:
(i) transmitting one or more instructions from an input device to the one or more processors of the robot controlling apparatus or actuator where the one or more instructions operate to move, control, bend, or change the state of the bendable robot or catheter; and
(ii) transmitting the one or more communications and/or transmitting the one or more instructions from the input device to the host processor of the host apparatus.

15. The method of claim 14, further comprising one or more of the following:
(i) generating, via the host processor, a Graphical User Interface (GUI) based on information of the one or more instructions from the input device, and/or displaying posture, position, orientation, bending, or other state information of the bendable robot or catheter;
(ii) generating, via the host processor, a Graphical User Interface (GUI) based on information of the one or more instructions from the input device, and/or displaying current posture, position, orientation, bending, or other state information of the bendable robot or catheter;
(iii) displaying a Graphical User Interface (GUI) to display posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately;
(iv) displaying a Graphical User Interface (GUI) to display current posture, position, orientation, bending, or other state information of the bendable robot or catheter such that: (a) one or more instructions of the input device are based on the posture, position, orientation, bending, or other state information of the bendable robot or catheter; and/or (b) the posture, position, orientation, bending, or other state information of the bendable robot or catheter are displayed to a user of the robot controlling apparatus or actuator such that the user determines, transmits, or updates one or more instructions of the input device accurately; and/or
(v) in the case where the error is detected in the one or more communications with the host processor, putting the robot controlling apparatus or actuator and/or the bendable robot or catheter in a safety shutdown mode to avoid misleading a user of the robot controlling apparatus or actuator and/or the bendable robot or catheter since the user cannot see result(s) of one or more instructions or actions of the user.

16. The method of claim 14, further comprising one or more of the following:
(i) sending, via the one or more processors, one or more of the following to the host processor of the host apparatus: state information of the bendable robot or catheter; state information of the bendable robot or catheter indicating whether the bendable robot or catheter is connected to the robot controlling apparatus or actuator or not; state information or navigation information of the bendable robot or catheter as a tip of the bendable robot or catheter and/or a linear stage connected to the bendable robot or catheter is or are manipulated, controlled, or moved; one or more requests from a user of the robot controlling apparatus or actuator or change the state of the bendable robot or catheter; one or more requests from the user to take one or more images or snapshots using a camera of the bendable robot or catheter; one or more requests from the user to rotate the one or more images or snapshots of the camera of the bendable robot or catheter; information for a warning indicating the error; information for a warning, failure, or error related to a voltage being out of a set or predetermined range, a temperature being out of a set or predetermined range, and/or an emergency stop; version information and parameter values; and/or information automatically sent to the host processor or information manually sent to the host processor in response to a request for the information;
(ii) sending, via the host processor, one or more of the following to the one or more processors of the robot controlling apparatus or actuator: one or more requests for state, version, voltage, temperature, and/or parameter information; one or more requests to home or to return to a default, predetermined, or rest position/state a linear stage being used to move the bendable robot or catheter; one or more requests to zero or reset one or more motors of the robot controlling apparatus or actuator that operate to change the state of the bendable robot or catheter; one or more requests to change the state of the bendable robot or catheter and/or the linear stage; information for a test or normal joystick mode; error report(s); a request for shutdown or stopping of the bendable robot or catheter and/or the robot controlling apparatus or actuator; and/or Electromagnetic Compatibility (EMC) testing parameters and operations; and/or
(iii) performing, via the host processor, one or more of the following: generation of a Graphical User Interface (GUI) based on information of the one or more instructions from the input device and/or to display current posture, position, orientation, bending, or other state information of the bendable robot or catheter; display of the GUI on a display; one or more pre-procedure operations related to login and/or entering case information; one or more procedure setup operations; one or more procedure operations related to displaying video from a camera of the bendable robot or catheter, showing the state or statue of the bendable robot or catheter, showing the state or statue of the bendable robot or catheter including one or more forces affecting one or more drive wires of the bendable robot or catheter, showing one or more warnings related to displaying the error information, and/or showing additional video feed(s); post-procedure operations related to reviewing cases and/or exporting the cases to a memory for storage; one or more management operations related to adding or removing user(s) of the robot controlling apparatus or actuator, the input device, and/or of the host apparatus, and/or related to handling one or more settings for each of the user(s); and/or writing or storing a log to a disk or to the memory.

17. The method of claim 15, using one or more indicators that operate to indicate that the error is happening or has happened, in response to the detection of the error in the one or more communications with the host processor, wherein one or more of the following:
(i) the one or more indicators independently or automatically signal that the error is happening or has happened, or the one or more indicators independently or automatically signal that the error is happening or has happened to a user of the robot controlling apparatus or actuator and/or the input device;
(ii) the one or more indicators are disposed in or on one or more of the following: the robot controlling apparatus or actuator, the input device, the host apparatus, and/or a display that operates to display a Graphical User Interface (GUI); and/or
(iii) the bendable robot or catheter, the input device, the host apparatus, and/or the robot controlling apparatus or actuator operates to independently signal that the error is happening or has happened via the one or more indicators as a primary or secondary signal of the error.

18. The method of claim 13, further comprising using or instructing one or more motors of the robot controlling apparatus or actuator to one or more of the following:
(i) move, control, bend, or change the state of the bendable robot or catheter; and/or
(ii) in the case where the error is detected in the one or more communications with the host processor and/or in a case where the one or more processors of the robot controlling apparatus or actuator do not receive a predetermined response and/or a valid message from the host processor of the host apparatus within a predetermined period or amount of time, restrict, disable, or stop the movement, control, bending, or changing of the state of the bendable robot or catheter, the predetermined response and/or the valid message including one or more of the following: a query version string; a request to reset the bendable robot or catheter; a request to run power-on test(s); a request or query for a cyclic redundancy check (CRC) and/or for an on or off status of the CRC; a parameter(s) query; a request to query, notify, or set a failure string; a request to query, notify, or set a warning string; a request to notify or log a string or log string to an application log; a request to shut down the bendable robot or catheter and/or the robot controlling apparatus or actuator; a request to start a new procedure; a request to power cycle; a request for a query of complete status; a request to query, notify or set a mode, a disconnect mode, a disabled mode, a halt mod, a tip control mode, a manual mode, a pause mode, a relax mode, a park mode, a Follow-the-Leader (FTL) mode, a reverse FTL mode, a targeting mode, and/or an error mode; a request to query or notify an emergency stop or E-stop, or to obtain an ok or tripped status of the emergency stop; a request to establish a home, zero, or default position for the bendable robot or catheter for one or more or all motors or drive wires of the robot controlling apparatus or actuator; a request to query or notify a connected or a disconnected status of the bendable robot or catheter; and/or a request for a wire test of the bendable robot or catheter.

19. The method of claim 13, wherein one or more of the following:
(i) the determination of whether the error is detected in the one or more communications with the host processor uses or communicates with one or more of the following: a driver or an operating system driver of the robot controlling apparatus or actuator, firmware of the robot controlling apparatus or actuator, software of the robot controlling apparatus or actuator, a robot state from the host processor of the host apparatus, and/or a serial communications and protocol manager of the host processor of the host apparatus;
(ii) the method further comprises determining the error is detected in the one or more communications with the host processor, and controlling an operation mode of the bendable robot or catheter to be in a relaxed mode and/or to be in a relaxed mode where the bendable robot or catheter bends or changes the state of the bendable robot or catheter based on or in accordance with an external force so that the bendable robot or catheter conforms to a lumen of an object, target, or sample in a case where the bendable robot or catheter is removed from the lumen of the object, target, or sample; and/or
(iii) the method further comprises determining the error is detected in the one or more communications with the host processor, and controlling an operation mode of the bendable robot or catheter to be in a relaxed mode such that the relaxed mode operates as a safety mode where the bendable robot or catheter is removed from an object, target, or sample.

20. The method of claim 13, further comprising moving or controlling a slider, a rail, and/or a translational stage attached to the robot controlling apparatus or actuator and/or the bendable robot or catheter to provide depth control or to remember a path along or into a lumen of a target, sample, or object, and one or more of the following:
(i) providing the depth control or remembering the path, and moving the bendable robot or catheter using one or more of the following: a Follow-The-Leader (FTL) algorithm or process to move one or more sections of the bendable robot or catheter along the path; and/or a reverse Follow-The-Leader (rFTL) algorithm or process to reverse the path exactly or to re-orient the one or more sections of the bendable robot or catheter in a manner in which there is less impedance in the bending used to align the bendable robot or catheter with the lumen of the target, sample, or object; and/or
(ii) detecting the error, stopping the movement of the robot controlling apparatus or actuator and/or the bendable robot or catheter via the slider, the rail, and/or the translational stage; or stopping the movement of the robot controlling apparatus or actuator and/or the bendable robot or catheter via the slider, the rail, and/or the translational stage such that a user of the robot controlling apparatus or actuator, of the host apparatus, and/or of the bendable robot or catheter is stopped or prevent from driving the bendable robot or catheter further into the lumen with no visibility or information of action(s) of the user.

21. The method of claim 20, further comprising one or more of the following:
(i) estimating or determining a depth of the depth control and/or estimating or determining the path to the target, sample, or object;
(ii) estimating or determining a depth of the depth control and/or estimating or determining the path to the target, sample, or object using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture; and/or
(iii) using a neural network, a convolutional neural network, a generative adversarial network (GAN), a consistent generative adversarial network (cGAN), a three cycle-consistent generative adversarial network (3cGAN), recurrent neural networks, and/or any other AI architecture to one or more of: use or evaluate an estimated or determined depth or path, identify one or more target points in the lumen or along the path, evaluate the accuracy of the identified one or more target points, plan the navigation of the bendable robot or catheter, autonomously move the bendable robot or catheter to the one or more target points, take one or more images, frames, or views using a camera of the bendable robot or catheter, and/or display the one or more target points on one of the one or more images, frames, or views on a display or indicate on the display or via an indicator on the display or via a light or indicator on or in communication with the bendable robot or catheter that the navigation has been planned or determined.

22. The method of claim 13, further comprising one or more of the following:
(i) inserting a camera into a lumen or disposing the camera at a distal end of the bendable robot or catheter to obtain one or more images or a live video feed for reference and/or for navigation in a lumen of an object, target, or sample;
(i) displaying the one or more images or the live video feed on a display; and/or
(ii) keeping the one or more images or the live video feed displayed, via the host processor, on a display even where the error is detected to allow for operation, movement, and/or removable of the bendable robot or catheter.

23. The method of claim 13, wherein one or more of the following:
(i) the bendable robot or catheter includes a distal bending section or portion, wherein the distal bending section or portion is commanded or instructed automatically or based on an input of a user of the bendable robot or catheter;
(ii) the bendable robot or catheter includes a plurality of bending sections or portions including a distal or most distal bending portion or section and the rest of the plurality of the bending sections or portions, where the plurality of bending sections or portions each include driving wires that operate to bend a respective section or portion of the plurality of sections or portions, and where the driving wires are connected to the robot controlling apparatus or actuator or to respective one or more motors of the robot controlling apparatus or actuator so that the robot controlling apparatus or actuator operates to bend one or more of the plurality of bending sections or portions using the driving wires; and/or
(iii) the robot controlling apparatus further comprises a slider, a rail, and/or a translational stage, and the one or more processors further operate to instruct or command the forward motion, or the motion in a set or predetermined direction, of the slider, the rail, the translational stage, and/or the bendable robot or catheter automatically or autonomously and/or based on an input of a user of the bendable robot or catheter.

24. A non-transitory computer-readable storage medium storing at least one program for causing a computer to execute a method for performing error detection or error removal for a robot controlling apparatus or actuator, the method comprising:
checking a status of one or more communications of the robot controlling apparatus or actuator with a host processor of a host apparatus;
determining whether an error is detected in the one or more communications with the host processor; and
in a case where the error is detected in the one or more communications with the host processor, stopping a bendable robot or catheter from moving, bending, or changing a state of the bendable robot or catheter.
